(19) United States
(12) Patent Application Publication
(10) Pub. No.: US 2007/0016976 A1
(43) Pub. Date: Jan. 18, 2007

(54) Plant genes involved in defense against pathogens

(76) Inventors: Fumiaki Katagiri, San Diego, CA (US); Yu-Ming Hou, San Diego, CA (US); Sheng Quan, San Diego, CA (US); Hur-Song Chang, San Diego, CA (US); Tong Zhu, San Diego, CA (US); Steve Whitham, San Diego, CA (US); Steve Goff, Encinitas, CA (US); Bret Cooper, La Jolla, CA (US); Jane Glazebrook, San Diego, CA (US); Wenquiong Chen, San Diego, CA (US); Zhiyi Xie, San Diego, CA (US); Yi Tao, La Jolla, CA (US); Guangzhou Zou, San Diego, CA (US)

Correspondence Address: SCHWEGMAN, LUNDBERG, WOESSNER & KLUTH, P.A., P.O. BOX 2938, MINNEAPOLIS, MN, 55402, US

(21) Appl. No.: 09/887,272

(22) Filed: Jun. 22, 2001

Related U.S. Application Data
(60) Provisional application No. 60/213,634, filed on Jun. 23, 2000.
(60) Provisional application No. 60/214,926, filed on Jun. 23, 2000.
(60) Provisional application No. 60/261,320, filed on Jan. 12, 2001.
(60) Provisional application No. 60/264,353, filed on Jan. 26, 2001.
(60) Provisional application No. 60/273,879, filed on Mar. 7, 2001.

(51) Int. Cl. A01H 5/00; C07H 21/04; C12N 5/04

(52) U.S. Cl. 800/279; 536/23.6; 435/320.1; 435/419

(57) ABSTRACT

Methods to identify genes, the expression of which are altered in response to pathogen infection, are provided, as well as the genes identified thereby and their corresponding promoters.

### CROSS-REFERENCE TO RELATED APPLICATIONS

This application claims the benefit of the filing date of U.S. application Ser. No. 60/213,634, filed on Jun. 23, 2000, U.S. application Ser. No. 60/214,926, filed on Jun. 23, 2000, U.S. application Ser. No. 60/261,320, filed on Jan. 12, 2001, U.S. application Ser. No. 60/264,353, filed on Jan. 26, 2001, and U.S. application Ser. No. 60/273,879, filed on Mar. 7, 2001 under 35 U.S.C. § 119(e).

### FIELD OF THE INVENTION

The present invention generally relates to the field of plant molecular biology, and more specifically to the regulation of gene expression in plants in response to pathogen exposure.

### BACKGROUND OF THE INVENTION

Plants are capable of activating a large array of defense mechanisms in response to pathogen attack, some of which are preexisting and others are inducible. Pathogens must specialize to circumvent the defense mechanisms of the host, especially those biotrophic pathogens that derive their nutrition from an intimate association with living plant cells. If the pathogen can cause disease, the interaction is said to be compatible, but if the plant is resistant, the interaction is said to be incompatible. A crucial factor determining the success of these mechanisms is the speed of their activation. Consequently, there is considerable interest in understanding how plants recognize pathogen attack and control expression of defense mechanisms.

Some potential pathogens trigger a very rapid resistance response called gene-for-gene resistance. This occurs when the pathogen carries an avirulence (avr) gene that triggers specific recognition by a corresponding host resistance (R) gene. R gene specificity is generally quite narrow, in most cases only pathogens carrying a particular avr gene are recognized. Recognition is thought to be mediated by ligand-receptor binding. R genes have been studied extensively in recent years. For a review of R genes, see Ellis et al. (1998); Jones et al. (1997); and Ronald (1998).

One of the defense mechanisms triggered by gene-for-gene resistance is programmed cell death at the infection site. This is called the hypersensitive response, or HR. Pathogens that induce the HR, or cause cell death by other means, activate a systemic resistance response called systemic acquired resistance (SAR). During SAR, levels of salicylic acid (SA) rise throughout the plant, defense genes such as pathogenesis related (PR) genes are expressed, and the plant becomes more resistant to pathogen attack. SA is a crucial component of this response. Plants that cannot accumulate SA due to the presence of a transgene that encodes an SA-degrading enzyme (nahG), develop a HR in response to challenge by avirulent pathogens, but do not exhibit systemic expression of defense genes and do not develop resistance to subsequent pathogen attack (Ryals et al., 1996). The nature of the systemic signal that triggers SAR is a subject of debate (Shulaev et al., 1995; Vemooji et al., 1994). SA clearly moves from the site of the HR to other parts of the plant, but if this is the signal, it must be effective at extremely low concentration (Willitset et al., 1998).

SAR is quite similar to some reactions that occur locally in response to attack by virulent (those that cause disease) or avirulent (those that trigger gene-for-gene resistance) pathogens. In general, activation of defense gene expression occurs more slowly in response to virulent pathogens than in response to avirulent pathogens. Some pathogens trigger expression of defense genes through a different signaling pathway that requires components of the jasmonic acid (JA) and ethylene signaling pathways (Creelman et al., 1997).

One approach to understanding the signal transduction networks that control defense mechanisms is to use genetic methods to identify signaling components and determine their roles within the network. Considerable progress has been made using this approach in Arabidopsis-pathogen model systems.

R Gene Signal Transduction

Genes such as NDR1 and EDS1, as well as DND1 and the lesion-mimic genes, likely act in signal transduction pathways downstream from R-avr recognition. NDR1 and EDS1 are required for gene-for-gene mediated resistance to avirulent strains of the bacterial pathogen Pseudomonas syringae and the oomycete pathogen Peronospora parasitica. Curiously, ndr1 mutants are susceptible to one set of avirulent pathogens, whereas eds1 mutants are susceptible to a non-overlapping set (Aarts et el., 1998). The five cloned R genes that require EDS1 all belong to the subset of the nucleotide binding site-leucine rich repeat (NBS-LRR) class of R genes that contain sequences similar to the cytoplasmic domains of Drosophila Toll and mammalian interleukin 1 transmembrane receptors (TIR-NBS-LRR). The two genes that require NDR1 belong to the leucine-zipper (LZ-NBS-LRR) subclass of NBS-LRR genes. There is another LZ-NBS-LRR gene, RPP8, that does not require EDS1 or NDR1, so the correlation between R gene structure and requirement for EDS1 or NDR1 is not perfect. Nevertheless, these results show that R genes differ in their requirements for downstream factors and that these differences are correlated with R gene structural type.

NDR1 encodes a protein with two predicted transmembrane domains (Century et al. 1997). RPM1, which requires NDR1 to mediate resistance, is membrane-associated, despite the fact that its primary sequence does not include any likely membrane-integral stretches (Boyes et al., 1998). It is possible that part of the function of NDR1 is to hold R proteins close to the membrane. EDS1 encodes a protein with blocks of homology to triacyl glycerol lipases (Falk et al., 1999). The significance of this homology is not known, but it is tempting to speculate that EDS1 is involved in synthesis or degradation of a signal molecule. EDS1 expression is inducible by SA and pathogen infection, suggesting that EDS1 may be involved in signal amplification (Falk et al., 1999).

It has been extremely difficult to isolate mutations in genes other than the R genes that are required for gene-for-gene resistance. A selection procedure was devised (McNellis et al., 1998) on the basis of precisely controlled inducible expression of the avr gene avrRpt2 in plants carrying the corresponding resistance gene RPS2. Expression of avrRpt2 in this background is lethal, as it triggers a systemic HR. It is now possible to select for mutants with subtle defects in gene-for-gene signaling by requiring growth on a concentration of inducer slightly higher than the lethal dose.

Putative plant receptor proteins encoded by RPP genes (recognition of P. parasitica) mediate specific recognition of Peronospora isolates and trigger defense reactions. Recently, McDowell et al. (2000) reported that two members of this class, RPP7 and RPP8 (the latter of which encodes a LZ-NBS-LRR type R protein) were not significantly suppressed by mutations in either EDS1 or NDR1, and that RPP7 resistance was also not compromised by mutations in EIN2, JAR1 or COI1, which affect ethylene or jasmonic acid signaling, or in coi1/npr1 or coi1/NahG backgrounds. The authors suggested that RPP7 initiates resistance through a novel signaling pathway that is independent of salicylic acid accumulation or jasmonic acid response components.

SA-Dependent Signaling

SA levels increase locally in response to pathogen attack, and systemically in response to the SAR-inducing signal. SA is necessary and sufficient for activation of PR gene expression and enhanced disease resistance. Physiological analyses and characterization of certain lesion-mimic mutants strongly suggest that there is a positive autoregulatory loop affecting SA concentrations (Shirasu et al., 1997; Hunt et al., 1997; Weymann et al., 1995). Several mutants with defects in SA signaling have been characterized. These include npr1, in which expression of PR genes in response to SA is blocked; cpr1, cpr5, and cpr6, which constitutively express PR genes; the npr1 suppressor ssi1; pad4, which has a defect in SA accumulation; and eds5, which has a defect in PR1 expression.

Expression of the defense genes PR1, BG2, and PR5 in response to SA treatment requires a gene called NPR1 or NIM1. Mutations in npr1 abolish SAR, and cause enhanced susceptibility to infection by various pathogens (Cao et al., 1994; Delaney et al., 1995; Glazebrook et al., 1996; Shah et al., 1997). NPR1 appears to be a positive regulator of PR gene expression that acts downstream from SA. NPR1 encodes a novel protein that contains ankyrin repeats (which are often involved in protein-protein interactions (Cao et al., 1997; Ryals et al., 1997), and that is localized to the nucleus in the presence of SA (Dong et al., 1998). Consequently, it is unlikely that NPR1 acts as a transcription factor to directly control PR gene expression, but its nuclear localization suggests that it may interact with such transcription factors.

PAD4 appears to act upstream from SA. In pad4 plants infected with a virulent P. syringae strain, SA levels, synthesis of the antimicrobial compound camalexin, and PR1 expression are all reduced (Zhou et al., 1998). SA is necessary, but not sufficient, for activation of camalexin synthesis (Zhou et al., 1998; Zhao et al., 1996). The camalexin defect in pad4 plants is reversible by exogenous SA (Zhou et al., 1998). Mutations in pad4 do not affect SA levels, camalexin synthesis, or PR1 when plants are infected with an avirulent P. syringae strain (Zhou et al., 1998). Taken together, these results suggest that PAD4 is required for signal amplification to activate the SA pathway in response to pathogens that do not elicit a strong defense response (Zhou et al., 1998).

JA-Dependent Signaling

JA signaling affects diverse processes including fruit ripening, pollen development, root growth, and response to wounding (Creelman et al., 1997). The jar1 and coi1 mutants fail to respond to JA (Feys et al., 1994; Staswick et al., 1992). COI1 has been cloned, and found to encode protein containing leucine-rich repeats and a degenerate F-box motif (Xie et al., 1998). These features are characteristic of proteins that function in complexes that ubiquitinate protein targeted for degradation.

In the past few years it has become apparent that JA plays an important role in regulation of pathogen defenses. For example, the induction of the defensin gene PDF1.2 after inoculation of Arabidopsis with the avirulent pathogen Alternaria brassicicola does not require SA or NPR1, but does require ethylene and JA signaling (Penninck et al., 1996).

SA signaling and JA signaling pathways are interconnected in complicated ways. Studies in other systems have shown that SA signaling and JA signaling are mutually inhibitory (Creelman et al., 1997; Harms et al., 1998). However, synthesis of camalexin in response to P. syringae infection is blocked in nahG (Zhou et al., 1998; Zhao et al., 1996) and coi1 (Glazebrook, 1999) plants, strongly suggesting that camalexin synthesis requires both SA and JA signaling.

Induced Systemic Resistance (ISR)

Some rhizosphere-associated bacteria promote disease resistance (van Loon et al., 1998). This phenomenon, called ISR, has been studied using Pseudomonas fluorescens strain WCS417r to colonize Arabidopsis roots (Pieterse et al., 1996). Colonized plants are more resistant to infection by the fungal pathogen Fusarium oxysporum f sp raphani and P. syringae (Pieterse et al., 1996). ISR occurs in nahG plants, indicating that it is not a SA-dependent phenomenon (Pieterse et al., 1996). Rather, ISR appears to be JA- and ethylene-dependent. The observation that ethylene can induce ISR in jar1 mutants led to the hypothesis that ISR requires a JA signal followed by an ethylene signal (Pieterse et al., 1998). No changes in gene expression associated with ISR have been detected (Pieterse et al., 1998), suggesting that it is different from activation of PDF1.2 expression by A. brassicicola.

Curiously, ISR requires NPR1 (Pieterse et al., 1996). This was unexpected in light of the fact that NPR1 was previously known to be involved only in SA-dependent processes and ISR is SA-independent. If the SA-dependent signal is received, NPR1 mediates a resistance response characterized by PR1 expression, whereas if the ISR signal is received, NPR1 mediates a different resistance response. It is difficult to imagine how this could occur, unless NPR1 is interacting with different ‘adapter’ molecules to mediate the different signals. The ankyrin repeats found in NPR1 could function in protein-protein interactions between NPR1 and adapter proteins. Identification of proteins that interact with NPR1, and characterization of plants with loss-of-function mutations affecting those proteins, would be very helpful for understanding how NPR1 acts in each pathway. It would also be worthwhile to determine if the ssi1 or cpr6 mutations suppress the ISR defect of npr1 mutants.

Relevance to Disease Resistance

Characterization of the effects of various mutations on resistance to different pathogens has revealed that there is considerable variation in the extent to which pathogens are affected by defense mechanisms. SAR is known to confer resistance to a wide array of pathogens, including bacteria, fungi, oomycetes, and viruses. JA signaling is important for limiting the growth of certain fungal pathogens. In Arabidopsis, the SA pathway mutants npr1 and pad4 show enhanced susceptibility to P. syringae and P. parasitica (Cao et al., 1994; Delaney et al., 1995; Shah et al., 1997; Zhou et al., 1998; Glazebrook et al., 1997).

Overexpression of rate-limiting defense response regulators may cause the signaling network to respond faster or more strongly to pathogen attack, thereby improving resistance. For example, overexpression of NPR1 caused increased resistance to P. syringae and P. parasitica in a dosage dependent manner (Cao et al., 1998). Moreover, NPR1-overexpression had no obvious deleterious effects on plant growth, in contrast to mutations that lead to constitutive overexpression of defense responses, which generally cause dwarfism.

Promoters for Gene Expression of Plant Pathogen Defense Genes

Promoters (and other regulatory components) from bacteria, viruses, fungi and plants have been used to control gene expression in plant cells. Numerous plant transformation experiments using DNA constructs comprising various promoter sequences fused to various foreign genes (for example, bacterial marker genes) have led to the identification of useful promoter sequences. It has been demonstrated that sequences up to 500-1,000 bases in most instances are sufficient to allow for the regulated expression of foreign genes. However, it has also been shown that sequences much longer than 1 kb may have useful features which permit high levels of gene expression in transgenic plants. The expression of genes encoding proteins that are useful for protecting plants from pathogen attack may have deleterious effects on plant growth if expressed constitutively. Consequently, it is desirable to have promoter sequences that control expression of these gene(s) in such a way that expression is absent or very low in the absence of pathogens, and high in the presence of pathogens.

Thus, what is needed is the identification of plant genes useful to confer resistance to a pathogen(s) and plant promoters, the expression of which is altered in response to pathogen attack.

### SUMMARY OF THE INVENTION

The invention generally provides an isolated nucleic acid molecule (polynucleotide) comprising a plant nucleotide sequence obtained or isolatable from a gene, the expression of which is altered, either increased or decreased, in response to pathogen infection. In one embodiment, the plant nucleotide sequence comprises an open reading frame, while in another embodiment, the plant nucleotide sequence comprises a promoter. A promoter sequence of the invention directs transcription of a linked nucleic acid segment, e.g., a linked plant DNA comprising an open reading frame for a structural or regulatory gene, in a host cell, such as a plant cell, in response to pathogen infection of that cell. As used herein, a “pathogen” includes bacteria, fungi, oomycetes, viruses, nematodes and insects, e.g., aphids (see Hammond-Kosack and Jones, 1997). Moreover, the expression of a plant nucleotide sequence of the invention comprising a promoter may be altered in response to one or more species of bacteria, nematode, fungi, oomycete, virus, or insect. Likewise, the expression of a plant nucleotide sequence of the invention comprising an open reading frame may be useful to confer tolerance or resistance of a plant to one or more species of bacteria, nematode, fungi, oomycete, virus or insect.

The nucleotide sequence preferably is obtained or isolatable from plant DNA. In particular, the nucleotide sequence is obtained or isolatable from a gene encoding a polypeptide which is substantially similar, and preferably has at least 70%, e.g., 71%, 72%, 73%, 74%, 75%, 76%, 77%, 78%, 79%, 80%, 81%, 82%, 83%, 84%, 85%, 86%, 87%, 88%, 89%, and even 90% or more, e.g., 91%, 92%, 93%, 94%, 95%, 96%, 97%, 98%, and 99%, amino acid sequence identity, to a polypeptide encoded by an Arabidopsis gene comprising any one of SEQ ID NOs: 1-953 and 2137-2661 or a fragment (portion) thereof which encodes a partial length polypeptide having substantially the same activity of the full-length polypeptide, a rice gene comprising one of SEQ ID NOs:2000-2129 and 2662-6813, or a Chenopodium gene comprising one of SEQ ID NOs:1954-1966.

The present invention also provides an isolated nucleic acid molecule comprising a plant nucleotide sequence that directs transcription of a linked nucleic acid segment in a host cell, e.g., a plant cell. The nucleotide sequence preferably is obtained or isolatable from plant genomic DNA. In particular, the nucleotide sequence is obtained or isolatable from a gene encoding a polypeptide which is substantially similar, and preferably has at least 70%, e.g., 71%, 72%, 73%, 74%, 75%, 76%, 77%, 78%, 79%, 80%, 81%, 82%, 83%, 84%, 85%, 86%, 87%, 88%, 89%, and even 90% or more, e.g., 91%, 92%, 93%, 94%, 95%, 96%, 97%, 98%, and 99%, amino acid sequence identity, to a polypeptide encoded by an Arabidopsis gene comprising any one of SEQ ID NOs:1-953 and 2137-2661, a rice gene comprising one of SEQ ID NOs:2000-2129 and 2662-6813, or a Chenopodium gene comprising any one of SEQ ID NOs:1954-1966, the expression of which is increased or decreased in response to pathogen infection. Preferred promoters comprise DNA obtained or isolatable from a gene encoding a polypeptide which is substantially similar, and preferably has at least 70%, e.g., 71%, 72%, 73%, 74%, 75%, 76%, 77%, 78%, 79%, 80%, 81%, 82%, 83%, 84%, 85%, 86%, 87%, 88%, 89%, and even 90% or more, e.g., 91%, 92%, 93%, 94%, 95%, 96%, 97%, 98%, and 99%, amino acid sequence identity, to a polypeptide encoded by an Arabidopsis gene comprising a promoter according to SEQ ID NOs:1001-1095 and 2137-2661, a rice gene comprising a promoter according to SEQ IN NOs:4738-6813, or a fragment thereof (i.e., promoters isolatable from any one of SEQ ID NOs:1001-1095, 2137-2661 and 4738-6813) which increases or decreases transcription of a linked nucleic acid segment in response to pathogen infection.

The invention also provides uses for an isolated nucleic acid molecule, e.g., DNA or RNA, comprising a plant nucleotide sequence comprising an open reading frame encoding a polypeptide which is substantially similar, and preferably has at least 70%, e.g., 71%, 72%, 73%, 74%, 75%, 76%, 77%, 78%, 79%, 80%, 81%, 82%, 83%, 84%, 85%, 86%, 87%, 88%, 89%, and even 90% or more, e.g., 91%, 92%, 93%, 94%, 95%, 96%, 97%, 98%, and 99%, amino acid sequence identity, to a polypeptide encoded by an Arabidopsis, Chenopodium or rice gene comprising an open reading frame comprising any one of SEQ ID NOs:1-953, 1954-1966, 2000-2129, 2662-4737 or the complement thereof. For example, these open reading frames may be useful to prepare plants that over- or under-express the encoded product or to prepare knockout plants.

The promoters and open reading frames of the invention can be identified by any method. For example, they can be identified by employing an array of nucleic acid samples, e.g., each sample having a plurality of oligonucleotides, and each plurality corresponding to a different plant gene, on a solid substrate, e.g., a DNA chip, and probes corresponding to nucleic acid which is up- or down-regulated in response to pathogen infection in one or more ecotypes or species of plant relative to a control (e.g., a water control, nucleic acid from an uninfected plant or nucleic acid from a mutant plant). Thus, genes that are upregulated or downregulated in response to pathogen infection can be systematically identified.

As described herein, GeneChip® technology was utilized to discover a plurality of genes, the expression of which is altered after pathogen infection. The Arabidopsis oligonucleotide probe array consists of probes from about 8,100 unique Arabidopsis genes, which covers approximately one third of the genome. This genome array permits a broader, more complete and less biased analysis of gene expression. Using labeled cRNA probes, expression levels were determined by laser scanning and genes generally selected for expression levels that were >2 fold over the control.

For example, using this approach, 953 genes were identified, the expression of which was altered after infection of wild-type Arabidopsis plants with a pathogen (SEQ ID NOs:1-953). In addition, 745 genes were identified, the expression which was increased after infection of wild-type Arabidopsis with Pseudomonas syringae (SEQ ID NOs: 2-6, 16, 18, 22-23, 25, 28-29, 31-32, 35-37, 39-43, 45-47, 49-50, 52, 54-55, 57-58, 60-66, 70-72, 74, 76-77, 79, 81, 83, 85, 87-90, 92, 94, 97, 100-107, 111-115, 117-125, 127-135, 138-140, 142-153, 156-158, 160, 162-165, 168-170, 173-181, 183-184, 186-188, 190-198, 200-201, 203-211, 214-215, 218-224, 227-232, 234-249, 251-262, 264, 266-268, 270, 272-275, 277-281, 283, 286-294, 297-298, 302, 304-306, 308-326, 328-339, 341, 344-345, 347, 350-351, 353-358, 361-371, 373-377, 379-386, 388-390, 392, 394-400, 402-406, 408-410, 412-417, 419-427, 429-433, 435-443, 445-452, 454-457, 459-460, 462-464, 466-470, 473-475, 478-479, 481-482, 484-187, 489-494, 496-498, 500-501, 503-506, 508, 510, 512-515, 517-523, 526, 528-529, 531-538, 540, 544-548, 550-558, 560, 563-568, 570, 572-577, 579-580, 582-585, 588-594, 596, 598-600, 602-603, 605-606, 608-612, 614-617, 619-624, 626-630, 632-639, 642, 644, 646-651, 653-657, 659-665, 667-671, 673-678, 681-689, 691-693, 695-713, 715-717, 719, 721-727, 729-733, 736-738, 740, 742, 744, 746, 748-752, 755-756, 758-760, 762-769, 771, 774, 776-781, 783-788, 790-796, 798-799, 802, 804-808, 810-815, 817-831, 833-848, 850-855, 857-869, 871-880, 882-900, 903-907, 909, 911-915, 918-920, 922-925, 927, 929, 931-938, 940, 943-945, 947, and 950-953). Of the 745 genes, the expression of 530 of those genes was altered in at least one mutant Arabidopsis after infection with Pseudomonas syringae (SEQ ID NOs: 2, 4-6, 11-13, 18, 22-23, 28, 31, 36, 39-43, 45, 47, 49-50, 52, 54-55, 57-58, 60-61, 63-66, 71-72, 74, 77, 81, 83, 85, 87-89, 92, 97, 100-107, 111-112, 114-115, 117-120, 122, 125, 127-128, 134, 128-140, 143-144, 148-151, 153, 156-157, 160, 165, 168-170, 173-174, 176-180, 183, 187-188, 191, 193-194, 197-198, 200, 203-210, 214, 219-224, 227, 230-232, 235-237, 239-240, 243-246, 248-249, 251-254, 256-258, 261, 264, 266-268, 270, 272-275, 277-278, 280, 283, 286-287, 290-293, 297, 302, 305-306, 308-310, 312-316, 321-326, 328-331, 333, 336-339, 341, 345, 351, 353, 355-358, 361-363, 365-366, 368-371, 373, 375, 377, 379-381, 384-385, 388-390, 392, 394-400, 402-406, 410, 412, 415-416, 419-420, 422-425, 429-433, 435-439, 441-443, 445-452, 454, 459-460, 463, 466, 468-470, 473, 481-482, 485-486, 489, 491-494, 497-498, 500-501, 503, 505-506, 508, 510, 513-515, 517, 520-521, 523, 528-529, 531, 533-538, 540, 545-548, 550-551, 553-554, 556-558, 560, 566-567, 575, 580, 582-584, 588-593, 596, 598-600, 602-603, 605-606, 608-610, 612, 614, 616, 620-622, 627-629, 633-634, 636-639, 644, 646, 648-651, 654-657, 659, 661-663, 667, 669, 673-674, 677, 682, 684-687, 689, 691-693, 697, 699, 701, 703-708, 713, 717, 719, 721-727, 730-733, 736, 740, 744, 746, 749-752, 755-756, 758-760, 762-764, 766-769, 774, 776-778, 780-781, 786, 788, 791-796, 799, 802, 804-808, 810-812, 815, 818-821, 823-825, 827-829, 831, 833-836, 838-843, 845, 847-848, 852-853, 855, 858, 860-869, 871-874, 876, 878-880, 884-887, 889, 892-894, 896-900, 904-907, 911-915, 918-920, 922-924, 931, 933, 938, 943-945, 947, and 950-952). Of the 530, 81 encode regulatory factors (SEQ ID NOs: 39, 52, 60, 63, 81, 83, 106, 107, 115, 117, 118, 168, 174, 176, 179, 204, 207, 208, 220, 221, 248, 258, 268, 275, 280, 309, 323, 326, 329, 351, 419, 422, 429, 430, 432, 459, 460, 468, 469, 473, 500, 505, 506, 508, 529, 531, 533, 535, 538, 545, 553, 602, 606, 608, 610, 614, 616, 634, 654, 655, 684, 686, 687, 691, 717, 751, 752, 766, 777, 815, 831, 834, 835, 839, 841, 847, 876, 884, 906, 920, and 924).

As also described herein, 333 genes were identified that are useful to confer improved resistance to plants to bacterial infection (SEQ ID NOs: 12-13, 18, 23, 36, 39-40, 43, 45, 50, 52, 57-58, 60-61, 64, 71-72, 81, 87-89, 97, 100, 102-105, 107, 111-112, 115, 119-120, 122, 125, 127-128, 140, 144, 148-150, 153, 165, 168-169, 176-177, 179, 183, 188, 191, 193-194, 197-198, 203-206, 208-209, 214, 219-222, 227, 230, 232, 237, 244-246, 248-249, 251-253, 258, 261, 264, 266, 268, 273-275, 283, 287, 290, 293, 297, 302, 305-306, 308, 312-315, 321-322, 324, 326, 330, 333, 338, 341, 345, 353, 356-358, 362-363, 366, 369, 371, 375, 377, 380, 384-385, 389, 392, 394-395, 398-399, 402-404, 406, 410, 415, 419, 422, 425, 429-430, 433, 435-439, 443, 445-452, 454, 463, 466, 468-470, 473, 486, 489, 491-492, 4894, 498, 500-501, 503, 508, 513-514, 517, 529, 533-538, 548, 550, 553-554, 4556-558, 566, 575, 580, 582-583, 590-591, 593, 600, 602, 609-610, 612, 614, 620-622, 627-629, 637-638, 644, 649, 654-657, 659, 663, 667, 669, 673-674, 677, 684-685, 689, 691-693, 699, 703-705, 708, 719, 721, 724-726, 730-732, 744, 746, 749-750, 752, 755-756, 758, 760, 762-764, 767, 769, 774, 780-781, 786, 788, 791-792, 794-796, 799, 804-808, 810-812, 815, 818-819, 823, 828-829, 833, 840841, 843, 847, 852-853, 858, 860, 862-865, 867-868, 872-874, 876, 885-887, 889, 892-894, 896-900, 904-905, 907, 911-914, 918-920, 922-924, 931, 933, 938, 947, 950, and 952).

Further, 296 genes were identified that are useful to confer improved resistance to plants to fungal infection (SEQ ID NOs: 2, 4, 6, 11-13, 18, 22-23, 31, 41-43, 49-50, 54, 57-58, 61, 64-66, 71-72, 74, 77, 85, 87, 89, 92, 97, 101, 103, 106-107, 112, 114, 117-119, 125, 128, 134, 138, 143, 149, 151, 156-157, 165, 169-170, 174, 176-180, 187-188, 191, 193, 206, 208, 219-220, 222, 224, 231, 236, 239, 243-245, 251-254, 256-257, 267, 272, 287, 290, 292, 297, 302, 312-313, 315-316, 321-322, 324-325, 328, 330, 345, 351, 353, 355-357, 362-363, 366, 368-371, 373, 375, 379, 381, 384, 388-390, 392, 395-400, 405, 410, 415-416, 419, 422, 424, 431-432, 435-436, 438-439, 447, 459-460, 470, 473, 481-482, 489, 491, 493-494, 500-501, 505-506, 513-514, 517, 520-521, 523, 528-529, 531, 535, 537-538, 540, 545-548, 551, 553-554, 557-558, 566, 575, 580, 582, 584, 589, 591, 593, 596, 598-599, 603, 605, 608-609, 612, 628, 633-634, 636-637, 639, 646, 648, 650-651, 656, 661, 663, 667, 674, 685-687, 689, 691, 693, 697, 699, 701, 705, 707, 713, 723-724, 726, 736, 740, 749, 751-752, 756, 758-759, 764, 766-768, 774, 776, 778, 780, 792-796, 799, 802, 806, 810-812, 818, 820-821, 825, 827-829, 833-836, 838-839, 841-843, 848, 855, 860-861, 866, 868-869, 871, 873-874, 876, 878-880, 889, 892, 898-900, 904-905, 907, 915, 918, 922, 924, 933, 943-945, 947, and 951).

In addition, 288 genes were identified that are useful to confer improved resistance to plants to infection with more than one pathogen, e.g., pathogens that include bacteria, oomycetes and viruses (SEQ ID NOs: 12-13, 18, 23, 36, 39-40, 43, 45, 50, 52, 57-58, 60-61, 64, 71-72, 81, 87-88, 100, 102-105, 107, 111-112, 115, 119-120, 122, 125, 127-128, 140, 148-150, 153, 168-169, 176-177, 188, 191, 193-194, 197-198, 203-206, 209, 219-222, 227, 232, 237, 244-246, 248-249, 251-253, 258, 261, 264, 266, 268, 273-275, 283, 287, 290, 293, 297, 302, 305-306, 308, 312-315, 324, 326, 330, 333, 341, 345, 353, 356, 358, 366, 371, 375, 377, 380, 385, 389, 392, 394, 398, 402-404, 406, 410, 415, 419, 425, 429-430, 433, 435-438, 443, 445-447, 449-452, 454, 463, 466, 468-470, 473, 486, 489, 492, 494, 498, 500-501, 503, 508, 513-514, 517, 533-538, 548, 550, 553-554, 57-558, 566, 575, 582-583, 590-591, 593, 600, 602, 609-610, 612, 620-622, 627-629, 637-638, 644, 649, 654-657, 659, 667, 669, 673, 677, 684, 689, 692-693, 703-705, 719, 721, 724-726, 730-732, 744, 746, 749-750, 752, 755-756, 760, 762-764, 767, 769, 774, 780-781, 786, 788, 791-792, 795-796, 805-808, 810-812, 815, 818-819, 823, 828, 833, 840-841, 843, 852-853, 858, 860, 862-865, 867-868, 872-874, 876, 887, 889, 893-894, 896-898, 900, 905, 907, 911-914, 918-920, 922-923, 931, 933, 938, 947, 950, and 952).

Using the same approach described above, 25 genes were identified (SEQ ID NOs: 1, 15, 19, 20, 24, 26, 27, 34, 38, 51, 56, 59, 67-69, 99, 116, 155, 159, 182, 212, 284, 372, 444, and 789), the expression of which was decreased at 6 hours in an avr2 plant. Also identified were 33 genes (SEQ ID NOs:17, 70, 76, 81, 84, 109, 123, 144, 160, 230, 265, 268, 269, 271, 323, 333, 385, 427, 428, 430, 457, 505, 569, 597, 602, 606, 616, 708, 730, 741, 812, 862, and 942), the expression of which was elevated in an incompatible or a compatible interaction in four Arabidopsis ecotypes infected with bacteria. Eight of the genes were upregulated by 3 hours in an incompatible interaction, 18 of the genes were upregulated by 6 hours, but not at 3 hours, in an incompatible interaction, and 6 of the genes were upregulated in a compatible interaction.

Further identified were 33 genes, the expression of which was induced early after infection (SEQ ID NOs:17, 21, 80, 81, 156, 174, 176, 221, 227, 296, 302, 303, 306, 333, 340, 360, 500, 505, 524, 575, 601, 602, 614, 628, 687, 733, 782, 811, 835, 862, 900, 905, and 912), 10 genes, the expression of which was decreased early after infection (SEQ ID NOs:30, 73, 282, 541, 640, 679, 761, 870, 917, and 930), and 135 genes, 107 of which were induced at 3 and/or 6 hours after infection, and 28 of which were decreased after infection (SEQ ID NOs:7, 21, 33, 44, 46, 60, 82, 86, 91, 93, 106, 110, 119, 122, 130, 131, 136, 141, 154, 161, 166-168, 171, 176, 185, 189, 199, 200, 202, 203, 213, 225, 227, 248, 261, 262, 266, 274, 285, 300, 301, 302, 320, 326, 341, 345, 348, 349, 360, 366, 378, 406, 409, 422, 425, 434, 441, 443, 446, 449, 454, 461, 471, 475, 476, 483, 485, 499, 500, 511, 512, 516, 527, 530, 533, 543, 545, 549, 550, 552, 567, 575, 578, 586, 590, 608, 611, 615, 618, 625, 631, 643, 656, 658, 659, 666, 668, 671, 680, 690, 694, 704, 706, 711, 714, 718, 721, 728, 734, 738, 757, 770, 772, 791, 807, 811, 813, 816, 827, 857, 864, 868, 875, 881, 893, 901, 905, 908, 912, 916, 939, 941, 951, and 952).

In a similar approach, 48 genes that were upregulated in response to infection, e.g., bacterial or fungal infection, as well as 46 of the corresponding promoter containing regions, were identified. Thirty-six of the genes were upregulated in response to bacterial, e.g., Pseudomonas, infection (the promoters for genes corresponding to SEQ ID NOs: 104-106, 119, 123, 129, 131, 151-152, 183, 191, 198, 200, 227, 249, 274, 302, 358, 415, 481, 547, 566, 582, 628, 633, 639, 656, 673, 793, 818, 827, 864, 874, 880, and 904-905), while 23 of the genes were upregulated in response to fungal, e.g., Botrykis, infection (SEQ ID NOs: 18, 71, 119, 123, 129, 151, 191, 244, 245, 302, 545, 547, 562, 566, 637, 653, 747, 756, 774, 793, 842, 864, and 905). Twenty-five of the genes were upregulated only in response to bacterial, e.g., Pseudomonas, infection (the promoters for genes corresponding to SEQ ID NOs: 104-106, 131, 152, 183, 198, 200, 227, 249, 274, 358, 415, 481, 582, 628, 633, 639, 656, 673, 818, 827, 874, 880, and 904 are provided in SEQ ID NOs:1001-1025), 10 of the genes were upregulated only in response to fungal, e.g., Botrytis, infection (the promoters for genes corresponding to SEQ ID NOs:18, 71, 244, 245, 545, 562, 637, 653, 747, 756, 774, and 842 are provided in SEQ ID NOs:1026-1035), and 11 genes were upregulated in response to both bacterial and fungal infection (the promoters for genes corresponding to SEQ ID NOs:119, 123, 129, 151, 191, 302, 547, 566, 793, 864, and 905 are provided in SEQ ID NOs:1036-1046).

As also described hereinbelow, 129 Arabidopsis genes (SEQ ID NOs: 3, 51, 54, 60, 61, 66, 75, 76, 78, 88, 95, 96, 101, 106, 108, 123, 126, 128, 129, 131, 137, 145-147, 150, 158, 169, 170, 172, 173, 197, 200, 216, 219, 224, 230, 233, 237, 249, 250, 263, 274, 275, 276, 299, 307, 323, 333, 342, 346, 359, 382, 383, 387, 391, 393, 401, 411, 415, 427, 442, 455, 459, 466, 477, 481, 485, 487, 502, 511, 515, 525, 534, 539, 542, 560, 571, 577, 579, 584, 587, 595, 600, 627, 638, 645, 654, 659, 668, 681, 688, 695, 696, 706, 708, 730, 742, 753, 775, 785, 786, 791, 797, 800, 801, 809, 817, 819, 820, 823, 827, 847, 856, 875, 885, 896, 902, 910, 921, 922, 923, 925, 926, 928, 946, and 952) were identified that were upregulated in response to viral infection, and 46 Arabidopsis genes were identified that were downregulated in response to viral infection (SEQ ID NOs: 14, 48, 53, 98, 217, 226, 295, 327, 343, 352, 369, 404, 407, 418, 453, 458, 465, 472, 480, 488, 495, 507, 509, 513, 514, 559, 561, 581, 604, 607, 613, 641, 652, 672, 720, 735, 739, 743, 745, 754, 773, 803, 832, 849, 948, and 949).

Also provided are nucleic acid molecules comprising a nucleotide sequence comprising an open reading frame expressed in response to pathogen infection comprising SEQ ID NOs:209, 216, 262, 267, 317, 386, 425, 440 and 800. These sequences are useful to over- or under-express the encoded product, or prepare knock-out plants which have an altered response to pathogen infection.

The invention therefore provides a method in which the open reading frame of a plant pathogen resistance gene, e.g., a gene that is associated with a response to pathogen infection, which is altered in a plant in response to infection is identified and isolated. A transgene comprising the isolated open reading frame may be introduced to and expressed in a transgenic plant, e.g., prior to infection, e.g., constitutively, or early and/or rapidly after infection, or in regulatable (inducible) fashion, e.g., after exposure to a chemical or using a promoter that is upregulated after infection, so as to confer resistance to that transgenic plant to the pathogen relative to a corresponding plant which does not have the transgene. The expression of the transgene is preferably at higher than normal levels, and under the regulation of a promoter that allows very fast and high induction in response to the presence of a pathogen or under cycling promoters (e.g., circadian clock regulated promoters), such that the encoded gene product(s) is maintained at sufficiently high levels to provide enhanced resistance or tolerance. The invention further provides a method in which a gene in a plant which is downregulated in response to infection, is disrupted or the expression of that gene is further downregulated, e.g., using antisense expression, so as to result in a plant that has enhanced resistance to infection, and which disruption or downregulation preferably has little or no detrimental effect(s) on the host plant.

As also described herein, it was found that the early incompatible response was similar to the late compatible response, suggesting that early expression of plant pathogen-resistance genes is important for resistance. Also, various plant strains were found to respond differently to the same pathogen, but there was also an identifiable global pattern of response. Thus, the comparison of the expression patterns in incompatible and compatible interactions in one or more ecotypes can lead to identifying subsets of key responding genes and clusters of genes that are key (early) responders. In addition, the observed global expression pattern indicated that the least resistant strain tested (Ws) had a low basal level of pathogen-upregulated genes and a high level of pathogen-downregulated genes compared to the most resistant strain (Ler). Thus, plant strains that are more resistant to pathogens have a gene expression phenotype in which genes that are upregulated in response to infection are already expressed at a higher than normal basal level, and those genes that are downregulated are expressed at a lower than normal basal level.

Thus, further provided herein is a method to identify at least one gene involved in plant (dicot or monocot) resistance or response to infection by at least one pathogen, e.g., a bacterium, fungus or virus, which method involves determining or detecting plant gene expression in an incompatible interaction and identifying at least one gene whose expression is significantly altered, e.g., upregulated or downregulated in response to infection, in the incompatible interaction relative to expression of the at least one gene in an uninfected plant, in a mutant plant that does not express a gene associated with response to infection by a pathogen, or in a corresponding compatible interaction. Also provided is a method to identify at least one gene involved in plant (dicot or monocot) resistance or response to infection by at least one pathogen, e.g., bacterium, fungus or virus, which method involves determining or detecting plant gene expression in a compatible interaction; and identifying at least one gene whose expression is significantly altered, e.g., upregulated or downregulated in response to infection, in the compatible interaction relative to expression of the at least one gene in an uninfected plant, in a mutant plant that does not express a gene associated with response to infection by a pathogen, or in a corresponding incompatible interaction. A compatible interaction can be, for example, between a plant having a resistance gene and a pathogen lacking a corresponding avirulence gene, a plant lacking a resistance gene to a pathogen having a corresponding avirulence gene, or a plant lacking a resistance gene and a pathogen lacking a corresponding avirulence gene. For example, the gene identified by such a method can encode a polypeptide that is substantially similar to a polypeptide encoded by an open reading frame comprising one of SEQ ID NOs: 50, 139, 609, 2699, 3311, 3463, 3584, 3791, 4210, 4451 or 4595, or has an open reading frame comprising one of SEQ ID NOs: 50, 139, 609, 2699, 3311, 3463, 3584, 3791, 4210, 4451 or 4595. In such a method, gene expression can be detected or determined using, for example, a gene chip, a cDNA array, cDNA-AFLP or differential display PCR. Such a method can further involve isolating the at least one gene or a portion thereof which includes the open reading frame or promoter for the gene.

Further provided is a method to identify at least one gene, the expression of which is altered by pathogen infection in a wild-type plant relative to a plant having a mutation that decreases jasmonic acid or ethylene-dependent signaling, which method involves contacting a plurality of isolated nucleic acid samples on a solid substrate each comprising isolated nucleic acid with a probe comprising plant nucleic acid corresponding to RNA from a wild-type plant infected with the pathogen, so as to form a complex, wherein each sample comprises a plurality of oligonucleotides corresponding to at least a portion of one plant gene; and comparing complex formation in a) with complex formation between a second plurality of isolated nucleic acid samples on a solid substrate with a second probe comprising nucleic acid corresponding to RNA from the plant having the mutation and infected with the pathogen, so as to identify a gene, the expression of which is altered by pathogen infection in a wild-type plant relative to the mutant plant. Also provided herein is a method to identify at least one gene, the expression of which is altered by pathogen infection in a wild-type plant relative to a plant having a mutation in a gene that interferes with salicylic acid dependent signaling, which method involves contacting a plurality of isolated nucleic acid samples on a solid substrate each comprising isolated nucleic acid with a probe comprising plant nucleic acid corresponding to RNA from a wild-type plant infected with the pathogen, so as to form a complex, wherein each sample comprises a plurality of oligonucleotides corresponding to at least a portion of one plant gene; and comparing complex formation in a) with complex formation between a second plurality of isolated nucleic acid samples on a solid substrate with a second probe comprising nucleic acid corresponding to RNA from the plant having a mutation and infected with the pathogen, so as to identify a gene, the expression of which is altered by pathogen infection in a wild-type plant relative to the mutant plant. Also provided herein is a method to identify at least one gene, the expression of which is altered by pathogen infection in a wild-type plant relative to a plant having a mutation that results in enhanced susceptibility to bacterial infection, which method involves contacting a plurality of isolated nucleic acid samples on a solid substrate each comprising isolated nucleic acid with a probe comprising plant nucleic acid corresponding to RNA from a wild-type plant infected with the pathogen, so as to form a complex, wherein each sample comprises a plurality of oligonucleotides corresponding to at least a portion of one plant gene; and comparing complex formation in a) with complex formation between a second plurality of isolated nucleic acid samples on a solid substrate with a second probe comprising nucleic acid corresponding to RNA from the plant having a mutation and infected with the pathogen, so as to identify a gene, the expression of which is altered by pathogen infection in a wild-type plant relative to the mutant plant. In addition, provided herein is a method to identify at least one gene, the expression of which is altered by infection with at least one virus, which method comprises contacting a plurality of isolated nucleic acid samples on a solid substrate each comprising isolated nucleic acid with a probe comprising plant nucleic acid corresponding to RNA from a wild-type plant infected with a virus, so as to form a complex, wherein each sample comprises a plurality of oligonucleotides corresponding to at least a portion of one plant gene; and comparing complex formation in a) with complex formation between a second plurality of isolated nucleic acid samples on a solid substrate with a second probe comprising nucleic acid corresponding to RNA from an uninfected plant, so as to identify a gene, the expression of which is altered by virus infection. Also provided is a method to identify at least one gene, the expression of which is altered by infection with at least one pathogen, which involves contacting a plurality of isolated nucleic acid samples on a solid substrate each comprising isolated nucleic acid with a probe comprising plant nucleic acid corresponding to RNA from an incompatible interaction so as to form a complex, wherein each sample comprises a plurality of oligonucleotides corresponding to at least a portion of one plant gene; and comparing complex formation in a) with complex formation between a second plurality of isolated nucleic acid samples on a solid substrate with a second probe comprising nucleic acid corresponding to RNA from a corresponding compatible interaction so as to identify a gene, the expression of which is altered by the pathogen. In any of the methods described herein, the probes can have nucleic acid, for example, from a dicot, a cereal plant, or a monocot. Further, the methods can additionally involve identifying the promoter for the at least one gene.

The genes and promoters described hereinabove can be used to identify orthologous genes and their promoters which are also likely useful to enhance resistance of plants to pathogens. Moreover, the orthologous promoters are useful to express linked open reading frames. In addition by aligning the promoters of these orthologs, novel cis elements can be identified that are useful to generate synthetic promoters.

Hence, the isolated nucleic acid molecules of the invention include the orthologs (homologs) of the Arabidopsis, Chenopodium and rice sequences disclosed herein, i.e., the corresponding nucleic acid molecules in organisms other than Arabidopsis, Chenopodium and rice, including, but not limited to, plants other than Arabidopsis, Chenopodium and rice, preferably cereal plants, e.g., corn, wheat, rye, turfgrass, sorghum, millet, sugarcane, soybean, barley, alfalfa, sunflower, canola, soybean, cotton, peanut, tobacco, sugarbeet, or rice. An ortholog is a gene from a different species that encodes a product having the same function as the product encoded by a gene from a reference organism. Databases such GenBank or one found at http://bioserver.myongjiac.kr/rjce.html (for rice) may be employed to identify sequences related to the Arabidopsis or Chenopodium sequences, e.g., orthologs in cereal crops such as rice. Alternatively, recombinant DNA techniques such as hybridization or PCR may be employed to identify sequences related to the Arabidopsis sequences. The encoded ortholog products likely have at least 70% sequence identity to each other. Hence, the invention includes an isolated nucleic acid molecule comprising a nucleotide sequence encoding a polypeptide having at least 70% identity to a polypeptide encoded by one or more of the Arabidopsis, Chenopodium or rice sequences disclosed herein. For example, promoter sequences within the scope of the invention are those which direct expression of an open reading frame which encodes a polypeptide that is substantially similar to an Arabidopsis polypeptide encoded by a gene comprising SEQ ID NOs:1-953.

The genes and promoters described hereinabove can be used to identify orthologous genes and their promoters which are also likely expressed in a particular tissue and/or development manner. Moreover, the orthologous promoters are useful to express linked open reading frames. In addition, by aligning the promoters of these orthologs, novel cis elements can be identified that are useful to generate synthetic promoters. Hence, the isolated nucleic acid molecules of the invention include the orthologs of the Arabidopsis sequences disclosed herein, i.e., the corresponding nucleotide sequences in organisms other than Arabidopsis, including, but not limited to, plants other than Arabidopsis, preferably cereal plants, e.g., corn, wheat, rye, turfgrass, sorghum, millet, sugarcane, soybean, barley, alfalfa, sunflower, canola, soybean, cotton, peanut, tobacco, sugarbeet, or rice. An orthologous gene is a gene from a different species that encodes a product having the same or similar function, e.g., catalyzing the same reaction as a product encoded by a gene from a reference organism. Thus, an ortholog includes polypeptides having less than, e.g., 65% amino acid sequence identity, but which ortholog encodes a polypeptide having the same or similar function. Databases such as GenBank or one found at http://bioserver.myongjiac.kr/rjce.html (for rice) may be employed to identify sequences related to the Arabidopsis sequences, e.g., orthologs in cereal crops such as rice, wheat, sunflower or alfalfa. SEQ ID NOs: 6286, 4210 and for example, are the rice promoter and open reading frame for rice peroxidase, the ortholog of the Arabidopsis gene comprising SEQ ID NO: 50. SEQ ID NOs: 3311, 5387, 3791 and 5867 are rice orthologs of the Arabidopsis gene comprising SEQ ID NO:609; SEQ ID NOs: 2699, 4775, 3463, 5539, 3584, 5660, 4451, 6527, 4595 and 6671 are rice orthologs of the Arabidopsis gene comprising SEQ ID NO: 139.

Preferably, the promoters of the invention include a consecutive stretch of about 25 to 2000, including 50 to 500 or 100 to 250, and up to 1000 or 1500, contiguous nucleotides, e.g., 40 to about 743, 60 to about 743, 125 to about 743, 250 to about 743, 400 to about 743, 600 to about 743, of any one of SEQ ID NOs:1001-1095, 2137-2661, 4738-6813 or the promoter orthologs thereof, which include the minimal promoter region. Preferably, the nucleotide sequence that includes the promoter region includes at least one copy of a TATA box. Thus, the invention provides plant promoters, including orthologs of Arabidopsis promoters corresponding to genes comprising any one of SEQ ID NOs: 1-953. The present invention further provides a composition, an expression cassette or a recombinant vector containing the nucleic acid molecule of the invention, and host cells comprising the expression cassette or vector, e.g., comprising a plasmid. In particular, the present invention provides an expression cassette or a recombinant vector comprising a promoter of the invention linked to a nucleic acid segment which, when present in a plant, plant cell or plant tissue, results in transcription of the linked nucleic acid segment.

In its broadest sense, the term “substantially similar” when used herein with respect to a nucleotide sequence means that the nucleotide sequence is part of a gene which encodes a polypeptide having substantially the same structure and function as a polypeptide encoded by a gene for the reference nucleotide sequence, e.g., the nucleotide sequence comprises a promoter from a gene that is the ortholog of the gene corresponding to the reference nucleotide sequence, as well as promoter sequences that are structurally related the promoter sequences particularly exemplified herein, i.e., the substantially similar promoter sequences hybridize to the complement of the promoter sequences exemplified herein under high or very high stringency conditions. The term “substantially similar” thus includes nucleotide sequences wherein the sequence has been modified, for example, to optimize expression in particular cells, as well as nucleotide sequences encoding a variant polypeptide comprising one or more amino acid substitutions relative to the (unmodified) polypeptide encoded by the reference sequence, which substitution(s) does not alter the activity of the variant polypeptide relative to the unmodified polypeptide. In its broadest sense, the term “substantially similar” when used herein with respect to polypeptide means that the polypeptide has substantially the same structure and function as the reference polypeptide. The percentage of amino acid sequence identity between the substantially similar and the reference polypeptide is at least 65%, 66%, 67%, 68%, 69%, 70%, e.g., 71%, 72%, 73%, 74%, 75%, 76%, 77%, 78%, 79%, 80%, 81%, 82%, 83%, 84%, 85%, 86%, 87%, 88%, 89%, and even 90% or more, e.g., 91%, 92%, 93%, 94%, 95%, 96%, 97%, 98%, up to at least 99%, wherein the reference polypeptide is a polypeptide encoded by an Arabidopsis gene comprising any one of SEQ ID NOs:1-953, a Chenopodium gene comprising any one of SEQ ID NOs:1954-1966, or a rice gene comprising any one of SEQ ID NOs:2000-2129 and 2662-4737. One indication that two polypeptides are substantially similar to each other, besides having substantially the same function, is that an agent, e.g., an antibody, which specifically binds to one of the polypeptides, specifically binds to the other.

Sequence comparisons maybe carried out using a Smith-Waterman sequence alignment algorithm (see e.g., Waterman (1995) or http://www hto.usc.edu/software/seqaln/index.html). The localS program, version 1.16, is preferably used with following parameters: match: 1, mismatch penalty: 0.33, open-gap penalty: 2, extended-gap penalty: 2. Further, a nucleotide sequence that is “substantially similar” to a reference nucleotide sequence hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, 1 mM EDTA at 50° C. with washing in 1×SSC, 0.1% SDS at 50° C., more desirably still in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5 M NaPO₄, 1 mM EDTA at 50° C. with washing in 0.1×SSC, 0.1% SDS at 65° C.

Hence, the present invention further provides an expression cassette or a vector containing the nucleic acid molecule comprising an open reading frame of the invention operably linked to a promoter, or comprising a promoter of the invention operably linked to an open reading frame or portion thereof, and the vector may be a plasmid. Such cassettes or vectors, when present in a plant, plant cell or plant tissue result in transcription of the linked nucleic acid fragment in the plant. The expression cassettes or vectors of the invention may optionally include other regulatory sequences, e.g., transcription terminator sequences, operator, repressor binding site, transcription factor binding site, and/or an enhancer and may be contained in a host cell. The expression cassette or vector may augment the genome of a transformed plant or may be maintained extrachromosomally. The expression cassette or vector may further have a Ti plasmid and be contained in an Agrobacterium tumefaciens cell; it may be carried on a microparticle, wherein the microparticle is suitable for ballistic transformation of a plant cell; or it may be contained in a plant cell or protoplast. Further, the expression cassette can be contained in a transformed plant or cells thereof and the plant may be a dicot or a monocot. In particular, the plant may be a cereal plant.

The invention also provides sense and anti-sense nucleic acid molecules corresponding to the open reading frames identified herein as well as their orthologs. Also provided are expression cassettes, e.g., recombinant vectors, and host cells, comprising the nucleic acid molecule of the invention, e.g., one which comprises a nucleotide sequence which encodes a polypeptide the expression of which is altered in response to pathogen infection.

The present invention further provides a method of augmenting a plant genome by contacting plant cells with a nucleic acid molecule of the invention, e.g., one isolatable or obtained from a plant gene encoding a polypeptide that is substantially similar to a polypeptide encoded by an Arabidopsis, Chenopodium or rice gene comprising a sequence comprising any one of SEQ ID NOs: 1-953, 1954-1966, 2000-2129 or 2662-4737 so as to yield transformed plant cells; and regenerating the transformed plant cells to provide a differentiated transformed plant, wherein the differentiated transformed plant expresses the nucleic acid molecule in the cells of the plant. The nucleic acid molecule may be present in the nucleus, chloroplast, mitochondria and/or plastid of the cells of the plant. The present invention also provides a transgenic plant prepared by this method, a seed from such a plant and progeny plants from such a plant including hybrids and inbreds. Preferred transgenic plants are transgenic maize, soybean, barley, alfalfa, sunflower, canola, soybean, cotton, peanut, sorghum, tobacco, sugarbeet, rice, wheat, rye, turfgrass, millet, sugarcane, tomato, or potato.

The invention also provides a method of plant breeding, e.g., to prepare a crossed fertile transgenic plant. The method comprises crossing a fertile transgenic plant comprising a particular nucleic acid molecule of the invention with itself or with a second plant, e.g., one lacking the particular nucleic acid molecule, to prepare the seed of a crossed fertile transgenic plant comprising the particular nucleic acid molecule. The seed is then planted to obtain a crossed fertile transgenic plant. The plant may be a monocot or a dicot. In a particular embodiment, the plant is a cereal plant.

The crossed fertile transgenic plant may have the particular nucleic acid molecule inherited through a female parent or through a male parent. The second plant may be an inbred plant. The crossed fertile transgenic may be a hybrid. Also included within the present invention are seeds of any of these crossed fertile transgenic plants.

The various breeding steps are characterized by well-defined human intervention such as selecting the lines to be crossed, directing pollination of the parental lines, or selecting appropriate progeny plants. Depending on the desired properties different breeding measures are taken. The relevant techniques are well known in the art and include but are not limited to hybridization, inbreeding, backcross breeding, multiline breeding, variety blend, interspecific hybridization, aneuploid techniques, etc. Hybridization techniques also include the sterilization of plants to yield male or female sterile plants by mechanical, chemical or biochemical means. Cross pollination of a male sterile plant with pollen of a different line assures that the genome of the male sterile but female fertile plant will uniformly obtain properties of both parental lines. Thus, the transgenic plants according to the invention can be used for the breeding of improved plant lines that for example increase the effectiveness of conventional methods such as herbicide or pesticide treatment or allow to dispense with said methods due to their modified genetic properties. Alternatively new crops with improved stress tolerance can be obtained that, due to their optimized genetic “equipment”, yield harvested product of better quality than products that were not able to tolerate comparable adverse developmental conditions.

The nucleic acid molecules of the invention, their encoded polypeptides and compositions thereof, are: for open reading frames, useful to provide resistance to pathogens to alter expression of a particular gene corresponding to the open reading frame by decreasing or eliminating expression of that plant gene or by overexpressing a particular gene product, and as a diagnostic for the presence or absence of the pathogen by correlating the expression level or pattern of expression of one or more of the nucleic acid molecules or polypeptides of the invention; and for promoters, useful to alter the expression of a linked open reading frame in response to pathogen infection. As one embodiment of the invention includes isolated nucleic acid molecules that have increased expression in response to pathogen infection, the invention further provides compositions and methods for enhancing resistance to pathogen infection. The compositions of the invention include plant nucleic acid sequences and the amino acid sequences for the polypeptides or partial-length polypeptides encoded thereby which are described herein, or other plant nucleic acid sequences and the amino acid sequences for the polypeptides or partial-length polypeptides encoded thereby which are operably linked to a promoters are useful to provide tolerance or resistance to a plant to a pathogen, preferably by preventing or inhibiting pathogen infection. Methods of the invention involve stably transforming a plant with one or more of at least a portion of these nucleotide sequences which confer tolerance or resistance operably linked to a promoter capable of driving expression of that nucleotide sequence in a plant cell. By “portion” or “fragment”, as it relates to a nucleic acid molecule, sequence or segment of the invention, when it is linked to other sequences for expression, is meant a sequence comprising at least 80 nucleotides, more preferably at least 150 nucleotides, and still more preferably at least 400 nucleotides. If not employed for expressing, a “portion” or “fragment” means at least 9, preferably 12, more preferably 15, even more preferably at least 20, consecutive nucleotides, e.g., probes and primers (oligonucleotides), corresponding to the nucleotide sequence of the nucleic acid molecules of the invention. By “resistant” is meant a plant which exhibits substantially no phenotypic changes as a consequence of infection with the pathogen. By “tolerant” is meant a plant which, although it may exhibit some phenotypic changes as a consequence of infection, does not have a substantially decreased reproductive capacity or substantially altered metabolism.

A method of combating a pathogen in an agricultural crop is also provided. The method comprises introducing to a plant, plant cell, or plant tissue an expression cassette comprising a nucleic acid molecule of the invention comprising an open reading frame so as to yield a transformed differentiated plant, transformed cell or transformed tissue. Transformed cells or tissue can be regenerated to provide a transformed differentiated plant. The transformed differentiated plant preferably expresses the nucleic acid molecule in an amount that confers resistance to the transformed plant to pathogen infection relative to a corresponding nontransformed plant. The present invention also provides a transformed plant prepared by the method, progeny and seed thereof. Examples of plant viruses which may be combated by the present invention include single stranded RNA viruses (with and without envelope), double stranded RNA viruses, and single and double stranded DNA viruses such as (but not limited to) tobacco mosaic virus, cucumber mosaic virus, turnip mosaic virus, turnip vein clearing virus, oilseed rape mosaic virus, tobacco rattle virus, pea enation mosaic virus, barley stripe mosaic virus, potato viruses X and Y, carnation latent virus, beet yellows virus, maize chlorotic virus, tobacco necrosis virus, turnip yellow mosaic virus, tomato bushy stunt virus, southern bean mosaic virus, barley yellow dwarf virus, tomato spotted wilt virus, lettuce necrotic yellows virus, wound tumor virus, maize streak virus, and cauliflower mosaic virus. Other pathogens within the scope of the invention include, but are not limited to, fungi such as Cochliobolus carbonum, Phytophthora infestans, Phytophthora sojae, Collesosichum, Melampsora lini, cladosporium fulvum, Heminthosporium maydia, Peronospora parasitica, Puccinia sorghi, and Puccinia polysora; bacteria such as Phynchosporium secalis, Pseudomonas glycinea, Xanthomonas oryzae and, Fusarium oxyaporium; and nematodes such as Globodera rostochiensis.

For example, the invention provides a nucleic acid molecule comprising a plant nucleotide sequence comprising at least a portion of a key effector gene(s) responsible for host resistance to particular pathogens. To provide resistance or tolerance to a pathogen in a plant, this sequence may be overexpressed individually, in the sense or antisense orientation, or in combination with other sequences to confer improved disease resistance or tolerance to a plant relative to a plant that does not comprise and/or express the sequence. The overexpression may be constitutive, or it may be preferable to express the effector gene(s) in a tissue-specific manner or from an inducible promoter including a promoter which is responsive to external stimuli, such as chemical application, or to pathogen infection, e.g., so as to avoid possible deleterious effects on plant growth if the effector gene(s) was constitutively expressed. In one embodiment of the invention, the promoter employed may be one that is rapidly and transiently and/or highly transcribed after pathogen infection.

A transformed (transgenic) plant of the invention includes plants, for example, a plant the cells of which have an expression cassette of the invention, i.e., an expression cassette having a polynucleotide of the invention operatively linked to an open reading frame, or, the genome of which is augmented by a nucleic acid molecule of the invention, or in which the corresponding gene has been disrupted, e.g., to result in a loss, a decrease or an alteration, in the function of the product encoded by the gene, which plant may also have increased yields, e.g., under conditions of pathogen infection, and/or produce a better-quality product than the corresponding wild-type plant. The nucleic acid molecules of the invention are thus useful for targeted gene disruption, as well as markers and probes.

For example, the invention includes a pathogen, e.g., virus, tolerant or resistant plant and seed thereof having stably integrated and expressed within its genome, a nucleic acid molecule of the invention. The normal fertile transformed (transgenic) plant may be selfed to yield a substantially homogenous line with respect to viral resistance or tolerance. Individuals of the line, or the progeny thereof, may be crossed with plants which optionally exhibit the trait. In a particular embodiment of the method, the selfing and selection steps are repeated at least five times in order to obtain the homogenous (isogenic) line. Thus, the invention also provides transgenic plants and the products of the transgenic plants.

The invention further includes a nucleotide sequence which is complementary to one (hereinafter “test” sequence) which hybridizes under low, moderate or stringent conditions with the nucleic acid molecules of the invention as well as RNA which is encoded by the nucleic acid molecule. When the hybridization is performed under stringent conditions, either the test or nucleic acid molecule of invention is preferably supported, e.g., on a membrane or DNA chip. Thus, either a denatured test or nucleic acid molecule of the invention is preferably first bound to a support and hybridization is effected for a specified period of time at a temperature of, e.g., between 55 and 70° C., in double strength citrate buffered saline (SC) containing 0.1% SDS followed by rinsing of the support at the same temperature but with a buffer having a reduced SC concentration. Depending upon the degree of stringency required such reduced concentration buffers are typically single strength SC containing 0.1% SDS, half strength SC containing 0.1% SDS and one-tenth strength SC containing 0.1% SDS.

The invention further provides a method to identify an open reading frame in the genome of a plant cell, the expression of which is altered by pathogen infection of that cell. The method comprises contacting a solid substrate comprising a plurality of samples comprising isolated plant nucleic acid of a probe comprising plant nucleic acid, e.g., cRNA, isolated from a pathogen infected plant so as to form a complex. Each individual sample comprises one or more nucleic acid sequences (e.g., oligonucleotides) corresponding to at least a portion of a plant gene. The method may be employed with nucleic acid samples and probes from any organism, e.g., any prokaryotic or eukaryotic organism. Preferably, the nucleic acid sample and probes are from a plant, such as a dicot or monocot. More preferably the nucleic acid samples and probes are from a cereal plant. Even more preferably the nucleic acids and probes are from a crop plant. A second plurality of samples on a solid substrate, i.e., a DNA chip, each comprising a plurality of samples comprising isolated plant nucleic acid is contacted with a probe comprising plant nucleic acid isolated from an uninfected or infected control (mutant) plant so as to form a complex. Then complex formation between the samples and probes comprising nucleic acid from infected or control cells compared. For example, potato virus X, tobacco mosaic virus, tobravirus, cucumber mosaic virus and gemnivirus are known to infect Arabidopsis. Thus, Arabidopsis genes, the expression of which is altered in response to infection by any of these viruses, can be identified. Regions that are 5′ to the start codon for the gene can then be identified and/or isolated.

The invention further provides a method for identifying a plant cell infected with a pathogen. The method comprises contacting nucleic acid obtained from a plant cell suspected of being infected with a pathogen with oligonucleotides corresponding to a portion of a plurality of sequences selected from SEQ ID NOs:1-953, 1954-1966, 2000-2129 or 2662-4737 under conditions effective to amplify those sequences. Then the presence of the amplified product is detected or determined. The presence of two or more amplified products, e.g., in an amount that is different than the amount of the corresponding amplified products from an uninfected plant, each corresponding to two or more SEQ ID NOs: 1-953, 1954-1966, 2000-2129 or 2662-4737 is indicative of pathogen infection.

The invention further provides a method for identifying a plant cell infected with a pathogen. The method comprises contacting a protein sample obtained from a plant cell suspected of being infected with a pathogen with an agent that specifically binds a polypeptide encoded by an open reading frame comprising SEQ ID NOs:1-953, 1954-1966, 2000-2129 or 2662-4737 so as to form a complex. Then the presence or amount of complex formation is detected or determined.

The invention provides an additional method for identifying a plant cell infected with a pathogen. The method comprises hybridizing a probe selected from SEQ ID NOs:1-953, 1954-1966, 2000-2129 or 2662-4737 to nucleic acid obtained from a plant cell suspected of being infected with a pathogen. The amount of the probe hybridized to nucleic acid obtained from a cell suspected of being infected with a virus is compared to hybridization of the probe to nucleic acid isolated from an uninfected cell. A change in the amount of at least two probes that hybridize to nucleic acid isolated from a cell suspected of being infected by a virus relative to hybridization of at least two probes to nucleic acid isolated from an uninfected cell is indicative of viral infection.

A method to shuffle the nucleic acids of the invention is provided. This method involves fragmentation of a nucleic acid corresponding to a nucleic acid sequence listed in SEQ ID NOs: 1-953, 1954-1966, 2000-2129 or 2662-4737, the orthologs thereof, and the corresponding genes, followed by religation. This method allows for the production of polypeptides having altered activity relative to the native form of the polypeptide. Accordingly, the invention provides cells and transgenic plants containing nucleic acid segments produced through shuffling that encode polypeptides having altered activity relative to the corresponding native polypeptide.

A computer readable medium, e.g., a magnetic tape, optical disk, CD-ROM, random access memory, volatile memory, non-volatile memory, or bubble memory, containing the nucleic acid sequences of the invention as well as methods of use for the computer readable medium are provided. For example, a computer readable medium can contain a nucleic acid molecule that has at least 70% nucleic acid sequence identity to SEQ ID NOs: 50, 139, 609, 4210, 6286, 3311, 5387, 3791, 5867, 2699, 4775, 3463, 5539, 3584, 5660, 4451, 6527, 4595, 6671 or the complement thereof. This medium allows a nucleic acid segment corresponding to a nucleic acid sequence listed in SEQ ID NOs:1-953, 1001-1095, 1954-1966, 2000-2129 or 2662-4737 to be used as a reference sequence to search against databases. This medium also allows for computer-based manipulation of a nucleic acid sequence corresponding to a nucleic acid sequence listed in SEQ ID NOs:1-953, 1001-1095, 1954-1966, 2000-2129 or 2662-4737, and the corresponding gene and polypeptide encoded by the nucleic acid sequence.

The invention also provides a method for marker-assisted breeding to select for plants having altered resistance to a pathogen. The method involves contacting plant DNA or cDNA with a probe corresponding to a nucleic acid sequence listed in SEQ ID NOs. 1-953, 1954-1966, 2000-2129 or 2662-4737, the orthologs thereof, and the corresponding genes, or a portion thereof which hybridizes under moderate stringency conditions to a gene corresponding to one of of SEQ ID Nos. 1-953, 1954-1966, 2000-2129 or 2662-4737 so as to form a duplex and detecting or determining the presence or amount of the duplex. The amount or presence of the duplex is indicative of the presence of a gene, the expression of which alters the resistance of the plant to a pathogen.

Therefore, another embodiment of the present invention provides a method of using known inducers or inhibitors of genes identified as being important in plant-pathogen interactions to induce genes that are important in resistance, or to inhibit genes that are downregulated in resistance.

Thus, some of the isolated nucleic acid molecules of the invention are useful in a method of combating a pathogen in an agricultural crop. The method comprises introducing to a plant an expression cassette comprising a nucleic acid molecule of the invention so as to yield a transformed differentiated plant. The transformed differentiated plant expresses the nucleic acid molecule in an amount that confers resistance to the transformed plant to infection relative to a corresponding nontransformed plant.

### DETAILED DESCRIPTION OF THE INVENTION

I. Definitions

The term “gene” is used broadly to refer to any segment of nucleic acid associated with a biological function. Thus, genes include coding sequences and/or the regulatory sequences required for their expression. For example, gene refers to a nucleic acid fragment that expresses mRNA or functional RNA, or encodes a specific protein, and which includes regulatory sequences. Genes also include nonexpressed DNA segments that, for example, form recognition sequences for other proteins. Genes can be obtained from a variety of sources, including cloning from a source of interest or synthesizing from known or predicted sequence information, and may include sequences designed to have desired parameters.

The term “native” or “wild type” gene refers to a gene that is present in the genome of an untransformed cell, i.e., a cell not having a known mutation.

A “marker gene” encodes a selectable or screenable trait.

The term “chimeric gene” refers to any gene that contains 1) DNA sequences, including regulatory and coding sequences, that are not found together in nature, or 2) sequences encoding parts of proteins not naturally adjoined, or 3) parts of promoters that are not naturally adjoined. Accordingly, a chimeric gene may comprise regulatory sequences and coding sequences that are derived from different sources, or comprise regulatory sequences and coding sequences derived from the same source, but arranged in a manner different from that found in nature.

A “transgene” refers to a gene that has been introduced into the genome by transformation and is stably maintained. Transgenes may include, for example, genes that are either heterologous or homologous to the genes of a particular plant to be transformed. Additionally, transgenes may comprise native genes inserted into a non-native organism, or chimeric genes. The term “endogenous gene” refers to a native gene in its natural location in the genome of an organism. A “foreign” gene refers to a gene not normally found in the host organism but that is introduced by gene transfer.

An “oligonucleotide” corresponding to a nucleotide sequence of the invention, e.g., for use in probing or amplification reactions, may be about 30 or fewer nucleotides in length (e.g., 9, 12, 15, 18, 20, 21 or 24, or any number between 9 and 30). Generally specific primers are upwards of 14 nucleotides in length. For optimum specificity and cost effectiveness, primers of 16 to 24 nucleotides in length may be preferred. Those skilled in the art are well versed in the design of primers for use processes such as PCR. If required, probing can be done with entire restriction fragments of the gene disclosed herein which may be 100's or even 1000's of nucleotides in length.

The terms “protein,” “peptide” and “polypeptide” are used interchangeably herein.

The nucleotide sequences of the invention can be introduced into any plant. The genes to be introduced can be conveniently used in expression cassettes for introduction and expression in any plant of interest. Such expression cassettes will comprise the transcriptional initiation region of the invention linked to a nucleotide sequence of interest. Preferred promoters include constitutive, tissue-specific, developmental-specific, inducible and/or viral promoters. Such an expression cassette is provided with a plurality of restriction sites for insertion of the gene of interest to be under the transcriptional regulation of the regulatory regions. The expression cassette may additionally contain selectable marker genes. The cassette will include in the 5′-3′ direction of transcription, a transcriptional and translational initiation region, a DNA sequence of interest, and a transcriptional and translational termination region functional in plants. The termination region may be native with the transcriptional initiation region, may be native with the DNA sequence of interest, or may be derived from another source. Convenient termination regions are available from the Ti-plasmid of A. tumefaciens, such as the octopine synthase and nopaline synthase termination regions. See also, Guerineau et al., 1991; Proudfoot, 1991; Sanfacon et al., 1991; Mogen et al., 1990; Munroe et al., 1990; Ballas et al., 1989; Joshi et al., 1987.

“Coding sequence” refers to a DNA or RNA sequence that codes for a specific amino acid sequence and excludes the non-coding sequences. It may constitute an “uninterrupted coding sequence”, i.e., lacking an intron, such as in a cDNA or it may include one or more introns bounded by appropriate splice junctions. An “intron” is a sequence of RNA which is contained in the primary transcript but which is removed through cleavage and re-ligation of the RNA within the cell to create the mature mRNA that can be translated into a protein.

The terms “open reading frame” and “ORF” refer to the amino acid sequence encoded between translation initiation and termination codons of a coding sequence. The terms “initiation codon” and “termination codon” refer to a unit of three adjacent nucleotides (‘codon’) in a coding sequence that specifies initiation and chain termination, respectively, of protein synthesis (mRNA translation).

A “functional RNA” refers to an antisense RNA, ribozyme, or other RNA that is not translated.

The term “RNA transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA” (mRNA) refers to the RNA that is without introns and that can be translated into protein by the cell. “cDNA” refers to a single- or a double-stranded DNA that is complementary to and derived from mRNA.

“Regulatory sequences” and “suitable regulatory sequences” each refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translation leader sequences, introns, and polyadenylation signal sequences. They include natural and synthetic sequences as well as sequences which may be a combination of synthetic and natural sequences. As is noted above, the term “suitable regulatory sequences” is not limited to promoters.

“5′ non-coding sequence” refers to a nucleotide sequence located 5′ (upstream) to the coding sequence. It is present in the fully processed mRNA upstream of the initiation codon and may affect processing of the primary transcript to mRNA, mRNA stability or translation efficiency (Turner et al., 1995).

“3′ non-coding sequence” refers to nucleotide sequences located 3′ (downstream) to a coding sequence and include polyadenylation signal sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al., 1989.

The term “translation leader sequence” refers to that DNA sequence portion of a gene between the promoter and coding sequence that is transcribed into RNA and is present in the fully processed mRNA upstream (5′) of the translation start codon. The translation leader sequence may affect processing of the primary transcript to mRNA, mRNA stability or translation efficiency.

The term “mature” protein refers to a post-translationally processed polypeptide without its signal peptide. “Precursor” protein refers to the primary product of translation of an mRNA. “Signal peptide” refers to the amino terminal extension of a polypeptide, which is translated in conjunction with the polypeptide forming a precursor peptide and which is required for its entrance into the secretory pathway. The term “signal sequence” refers to a nucleotide sequence that encodes the signal peptide.

The term “intracellular localization sequence” refers to a nucleotide sequence that encodes an intracellular targeting signal. An “intracellular targeting signal” is an amino acid sequence that is translated in conjunction with a protein and directs it to a particular sub-cellular compartment. “Endoplasmic reticulum (ER) stop transit signal” refers to a carboxy-terminal extension of a polypeptide, which is translated in conjunction with the polypeptide and causes a protein that enters the secretory pathway to be retained in the ER. “ER stop transit sequence” refers to a nucleotide sequence that encodes the ER targeting signal. Other intracellular targeting sequences encode targeting signals active in seeds and/or leaves and vacuolar targeting signals.

“Pathogen” as used herein includes but is not limited to bacteria, fungi, yeast, oomycetes and virus, e.g., American wheat striate mosaic virus mosaic (AWSMV), barley stripe mosaic virus (BSMV), barley yellow dwarf virus (BYDV), Brome mosaic virus (BMV), cereal chlorotic mottle virus (CCMV), corn chlorotic vein banding virus (CCVBV), maize chlorotic mottle virus (MCMV), maize dwarf mosaic virus (MDMV), A or B, wheat streak mosaic virus (WSMV), cucumber mosaic virus (CMV), cynodon chlorotic streak virus (CCSV), Johnsongrass mosaic virus (JGMV), maize bushy stunt or mycoplasma-like organism (N]ILO), maize chlorotic dwarf virus (MCDV), maize chlorotic mottle virus (MCMV), maize dwarf mosaic virus (MDMV) strains A, D, E and F, maize leaf fleck virus (MLFV), maize line virus (NELV), maize mosaic virus (MMV), maize mottle and chlorotic stunt virus, maize pellucid ringspot virus (MPRV), maize raya gruesa virus (MRGV), maize rayado fino virus (MRFV), maize red leaf and red stripe virus (MRSV), maize ring mottle virus (MRMV), maize rio cuarto virus (MRCV), maize rough dwarf virus (MRDV), maize sterile stunt virus (strains of barley yellow striate virus), maize streak virus (MSV), maize chlorotic stripe, maize hoja Maize stripe virus blanca, maize stunting virus, maize tassel abortion virus (MTAV), maize vein enation virus (MVEV), maize wallaby ear virus (MAVEV), maize white leaf virus, maize white line mosaic virus (NTVVLMV), millet red leaf virus (NMV), Northern cereal mosaic virus (NCMV), oat pseudorosette virus, oat sterile dwarf virus (OSDV), rice black-streaked dwarf virus (RBSDV), rice stripe virus (RSV), sorghum mosaic virus (SrMV), formerly sugarcane mosaic virus (SCMV) strains H, I and M, sugarcane Fiji disease virus (FDV), sugarcane mosaic virus (SCMV) strains A, B, D, E, SC, BC, Sabi and NM vein enation virus, and wheat spot mosaic virus (WSMV).

Bacterial pathogens include but are not limited to Pseudomonas avenae subsp. avenae, Xanthomonas campestris pv. holcicola, Enterobacter dissolvens, Erwinia dissolvens, Ervinia carotovora subsp. carotovora, Erwinia chrysanthemi pv. zeae, Pseudomonas andropogonis, Pseudomonas syringae pv. coronafaciens, Clavibacter michiganensis subsp., Corynebacterium michiganense pv. nebraskense, Pseudomonas syringae pv. syringae, Hemiparasitic bacteria (see under fungi), Bacillus subtilis, Erwinia stewartii, and Spiroplasma kunkelii.

Fungal pathogens include but are not limited to Collelotrichum graminicola, Glomerella graminicola Politis, Glomerella lucumanensis, Aspergillus flavus, Rhizoctonia solani Kuhn, Thanatephorus cucumeris, Acremonium strictum W. Gams, Cephalosporium acremonium Auct. non Corda Black Lasiodiplodia theobromae=BoIr odiplodia y theobromae Borde blanco Marasmiellus sp., Physoderma maydis, Cephalosporium Corticium sasakii, Curvularia clavata, C. maculans, Cochhobolus eragrostidis, Curvularia inaequahs, C. intermedia (teleomorph Cochhobolus intermedius), Curvularia lunata (teleomorph: Cochliobolus lunatus), Curvularia pallescens (teleomorph—Cochliobolus pallescens), Curvularia senegalensis, C. luberculata (teleomorph: Cochliobolus tuberculatus), Didymella exitalis Diplodiaftumenti (teleomorph—Botryosphaeriafestucae), Diplodia maydis=Stenocarpella maydis, Stenocarpella macrospora=Diplodia macrospora, Sclerophthora rayssiae var. zeae, Sclerophthora macrospora=Sclerospora macrospora, Sclerospora graminicola, Peronosclerospora maydis=Sclerospora maydis, Peronosclerospora philippinensis, Sclerospora philippinensis, Peronosclerospora sorghi=Sclerospora sorghi, Peronosclerospora spontanea=Sclerospora spontanea, Peronosclerospora sacchari=Sclerospora sacchari, Nigrospora oryzae (teleomorph: Khuskia oryzae) A. Iternaria alternala=A. tenuis, Aspergillus glaucus, A. niger, Aspergillus spp., Botrytis cinerea, Cunninghamella sp., Curvulariapallescens, Doratomyces slemonitis=Cephalotrichum slemonitis, Fusarium culmorum, Gonatobotrys simplex, Pithomyces maydicus, Rhizopus microsporus Tiegh., R. stolonifer=R. nigricans, Scopulariopsis brumptii, Claviceps gigantea (anamorph: Sphacelia sp) Aureobasidium zeae=Kabatiella zeae, Fusarium subglutinans=F. moniliforme var. subglutinans, Fusarium moniliforme, Fusarium avenaceum (teleomorph—Gibberella avenacea), Botryosphaeria zeae=Physalospora zeae (anamorph: Allacrophoma zeae), Cercospora sorghi=C. sorghi var. maydis, Helminthosporium pedicellatum (teleomorph: Selosphaeriapedicellata), Cladosporium cladosporioides=Hormodendrum cladosporioides, C. herbarum (teleomorph—Mycosphaerella tassiana), Cephalosporium maydis, A. Iternaria alternata, A. scochyta maydis, A. tritici, A. zeicola, Bipolaris victoriae, Helminthosporium victoriae (teleomorph Cochhoholus victoriae), C sativus (anamorph: Bipolaris sorokiniana=H. sorokinianum=H. sativum), Epicoccum nigrum, Exserohilum prolatum=Drechslera prolata (teleomorph: Setosphaeriaprolata), Graphium penicillioides, Leptosphaeria maydis, Leptothyrium zeae, Ophiosphaerella herpotricha (anamorph—Scolecosporiella sp.), Pataphaeosphaeria michotii, Phoma sp., Septoria zeae, S. zeicola, S. zeina Setosphaeria turcica, Exserohilzim turcicum=Helminthosporium furcicum, Cochhoholus carbonum, Bipolaris zeicola=Helminthosporium carhonum, Penicilhum spp., P. chrysogenum, P. expansum, P. oxalicum, Phaeocytostroma ambiguum, Phaeocylosporella zeae, Phaeosphaeria maydis=Sphaerulina maydis, Botryosphaeriafestucae=Physalospora zeicola (anamorph: Diplodiaftumenfi), Herniparasitic bacteria and fungi Pyrenochaeta Phoma terrestris=Pyrenochaeta terrestris, Pythium spp., P. arrhenomanes, P. graminicola, Pythium aphanidermatum=P. hutleri L., Rhizoctonia zeae (teleomorph: Waitea circinata), Rhizoctonia solani, minor A Iternaria alternala, Cercospora sorghi, Dictochaetaftrtilis, Fusarium acuminatum (teleomorph Gihherella acuminata), E. equiseti (teleomorph: G. intricans), E. oxysporum, E. pallidoroseum, E. poae, E. roseum, G. cyanogena (anamorph: E. sulphureum), Microdochium holleyi, Mucor sp., Periconia circinata, Phytophthora cactorum, P. drechsleri, P. nicotianae var. parasitica, Rhizopus arrhizus, Setosphaeria rostrata, Exserohilum rostratum=Helminthosporium rostratum, Puccinia sorghi, Physopella pallescens, P. zeae, Sclerotium rofsii Sacc. (teleomorph—Athelia rotfsii), Bipolaris sorokiniana, B. zeicola=Helminthosporium carbonum, Diplodia maydis, Exserohilum pedicillatum, Exserohilum furcicum=Helminthosporium turcicum, Fusarium avenaceum, E. culmorum, E. moniliforme, Gibberella zeae (anamorph—E. graminearum), Macrophominaphaseolina, Penicillium spp., Phomopsis sp., Pythium spp., Rhizoctonia solani, R. zeae, Sclerotium rolfsfi, Spicaria sp., Selenophoma sp., Gaeumannomyces graminis, Myrothecium gramineum, Monascus purpureus, M ruber Smut, Ustilago zeae=U. maydis Smut, Ustilaginoidea virens Smut, Sphacelotheca reiliana=Sporisorium holci, Cochliobolus heterostrophus (anamorph: Bipolaris maydis=Helminthosporium maydis), Stenocarpella macrospora=Diplodia macrospora, Cercospora sorghi, Fusarium episphaeria, E. merismoides, F. oxysporum Schlechtend, E. poae, E. roseum, E. solani (teleomorph: Nectria haematococca), F. tricincturn, Mariannaea elegans, Mucor sp., Rhopographus zeae, Spicaria sp., Aspergillus spp., Penicillium spp., Trichoderma viride=T lignorum teleomorph: Hypocrea sp., Stenocarpella maydis=Diplodia zeae, Ascochyta ischaemi, Phyllosticta maydis (telomorph: Mycosphaerella zeae-maydis), and Gloeocercospora sorghi.

Parasitic nematodes include but are not limited to Awl Dolichodorus spp., D. heterocephalus Bulb and stem (Europe), Ditylenchus dipsaci Burrowing Radopholus similis Cyst Heterodera avenae, H. zeae, Punctodera chalcoensis Dagger Xiphinema spp., X americanum, X mediterraneum False root-knot Nacobbus dorsalis Lance, Columbia Hoplolaimus columbus Lance Hoplolaimus spp., H. galeatus Lesion Pratylenchus spp., P. brachyurus, P. crenalus, P. hexincisus, P. neglectus, P. penetrans, P. scribneri, P. thornei, P. zeae Needle Longidorus spp., L. breviannulatus Ring Criconemella spp., Cornata Root-knot Meloidogyne spp., M. chitwoodi, M. incognita, M. javanica Spiral Helicotylenchus spp., Belonolaimus spp., B. longicaudatus Stubby-root Paratrichodorus spp., P. christiei, P. minor, Ouinisulcius aculus, and Trichodorus spp.

“Promoter” refers to a nucleotide sequence, usually upstream (5′) to its coding sequence, which controls the expression of the coding sequence by providing the recognition for RNA polymerase and other factors required for proper transcription. “Promoter” includes a minimal promoter that is a short DNA sequence comprised of a TATA box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence which can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Both enhancers and other upstream promoter elements bind sequence-specific DNA-binding proteins that mediate their effects. Promoters may be derived in their entirety from a native gene, or be composed of different elements derived from different promoters found in nature, or even be comprised of synthetic DNA segments. A promoter may also contain DNA sequences that are involved in the binding of protein factors which control the effectiveness of transcription initiation in response to physiological or developmental conditions.

The “initiation site” is the position surrounding the first nucleotide that is part of the transcribed sequence, which is also defined as position +1. With respect to this site all other sequences of the gene and its controlling regions are numbered. Downstream sequences (i.e., further protein encoding sequences in the 3′ direction) are denominated positive, while upstream sequences (mostly of the controlling regions in the 5′ direction) are denominated negative.

Promoter elements, particularly a TATA element, that are inactive or that have greatly reduced promoter activity in the absence of upstream activation are referred to as “minimal or core promoters.” In the presence of a suitable transcription factor, the minimal promoter functions to permit transcription. A “minimal or core promoter” thus consists only of all basal elements needed for transcription initiation, e.g., a TATA box and/or an initiator.

“Constitutive expression” refers to expression using a constitutive or regulated promoter. “Conditional” and “regulated expression” refer to expression controlled by a regulated promoter.

“Constitutive promoter” refers to a promoter that is able to express the open reading frame (ORF) that it controls in all or nearly all of the plant tissues during all or nearly all developmental stages of the plant. Each of the transcription-activating elements do not exhibit an absolute tissue-specificity, but mediate transcriptional activation in most plant parts at a level of ≧1% of the level reached in the part of the plant in which transcription is most active.

“Regulated promoter” refers to promoters that direct gene expression not constitutively, but in a temporally- and/or spatially-regulated manner, and includes both tissue-specific and inducible promoters. It includes natural and synthetic sequences as well as sequences which may be a combination of synthetic and natural sequences. Different promoters may direct the expression of a gene in different tissues or cell types, or at different stages of development, or in response to different environmental conditions. New promoters of various types useful in plant cells are constantly being discovered, numerous examples may be found in the compilation by Okamuro et al. (1989). Typical regulated promoters useful in plants include but are not limited to safener-inducible promoters, promoters derived from the tetracycline-inducible system, promoters derived from salicylate-inducible systems, promoters derived from alcohol-inducible systems, promoters derived from glucocorticoid-inducible system, promoters derived from pathogen-inducible systems, and promoters derived from ecdysome-inducible systems.

“Tissue-specific promoter” refers to regulated promoters that are not expressed in all plant cells but only in one or more cell types in specific organs (such as leaves or seeds), specific tissues (such as embryo or cotyledon), or specific cell types (such as leaf parenchyma or seed storage cells). These also include promoters that are temporally regulated, such as in early or late embryogenesis, during fruit ripening in developing seeds or fruit, in fully differentiated leaf, or at the onset of senescence.

“Inducible promoter” refers to those regulated promoters that can be turned on in one or more cell types by an external stimulus, such as a chemical, light, hormone, stress, or a pathogen.

“Operably-linked” refers to the association of nucleic acid sequences on single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

“Expression” refers to the transcription and/or translation of an endogenous gene, ORF or portion thereof, or a transgene in plants. For example, in the case of antisense constructs, expression may refer to the transcription of the antisense DNA only. In addition, expression refers to the transcription and stable accumulation of sense (mRNA) or functional RNA. Expression may also refer to the production of protein.

“Specific expression” is the expression of gene products which is limited to one or a few plant tissues (spatial limitation) and/or to one or a few plant developmental stages (temporal limitation). It is acknowledged that hardly a true specificity exists: promoters seem to be preferably switch on in some tissues, while in other tissues there can be no or only little activity. This phenomenon is known as leaky expression. However, with specific expression in this invention is meant preferable expression in one or a few plant tissues.

The “expression pattern” of a promoter (with or without enhancer) is the pattern of expression levels which shows where in the plant and in what developmental stage transcription is initiated by said promoter. Expression patterns of a set of promoters are said to be complementary when the expression pattern of one promoter shows little overlap with the expression pattern of the other promoter. The level of expression of a promoter can be determined by measuring the ‘steady state’ concentration of a standard transcribed reporter mRNA. This measurement is indirect since the concentration of the reporter mRNA is dependent not only on its synthesis rate, but also on the rate with which the mRNA is degraded. Therefore, the steady state level is the product of synthesis rates and degradation rates.

The rate of degradation can however be considered to proceed at a fixed rate when the transcribed sequences are identical, and thus this value can serve as a measure of synthesis rates. When promoters are compared in this way techniques available to those skilled in the art are hybridization S1-RNAse analysis, northern blots and competitive RT-PCR. This list of techniques in no way represents all available techniques, but rather describes commonly used procedures used to analyze transcription activity and expression levels of mRNA.

The analysis of transcription start points in practically all promoters has revealed that there is usually no single base at which transcription starts, but rather a more or less clustered set of initiation sites, each of which accounts for some start points of the mRNA. Since this distribution varies from promoter to promoter the sequences of the reporter mRNA in each of the populations would differ from each other. Since each mRNA species is more or less prone to degradation, no single degradation rate can be expected for different reporter mRNAs. It has been shown for various eukaryotic promoter sequences that the sequence surrounding the initiation site (‘initiator’) plays an important role in determining the level of RNA expression directed by that specific promoter. This includes also part of the transcribed sequences. The direct fusion of promoter to reporter sequences would therefore lead to suboptimal levels of transcription.

A commonly used procedure to analyze expression patterns and levels is through determination of the ‘steady state’ level of protein accumulation in a cell. Commonly used candidates for the reporter gene, known to those skilled in the art are β-glucuronidase (GUS), chloramphenicol acetyl transferase (CAT) and proteins with fluorescent properties, such as green fluorescent protein (GFP) from Aequora victoria. In principle, however, many more proteins are suitable for this purpose, provided the protein does not interfere with essential plant functions. For quantification and determination of localization a number of tools are suited. Detection systems can readily be created or are available which are based on, e.g., immunochemical, enzymatic, fluorescent detection and quantification. Protein levels can be determined in plant tissue extracts or in intact tissue using in situ analysis of protein expression.

Generally, individual transformed lines with one chimeric promoter reporter construct will vary in their levels of expression of the reporter gene. Also frequently observed is the phenomenon that such transformants do not express any detectable product (RNA or protein). The variability in expression is commonly ascribed to ‘position effects’, although the molecular mechanisms underlying this inactivity are usually not clear.

The term “average expression” is used here as the average level of expression found in all lines that do express detectable amounts of reporter gene, so leaving out of the analysis plants that do not express any detectable reporter mRNA or protein.

“Root expression level” indicates the expression level found in protein extracts of complete plant roots. Likewise, leaf, and stem expression levels, are determined using whole extracts from leaves and stems. It is acknowledged however, that within each of the plant parts just described, cells with variable functions may exist, in which promoter activity may vary.

“Non-specific expression” refers to constitutive expression or low level, basal (‘leaky’) expression in nondesired cells or tissues from a ‘regulated promoter’.

“Altered levels” refers to the level of expression in transgenic organisms that differs from that of normal or untransformed organisms.

“Overexpression” refers to the level of expression in transgenic cells or organisms that exceeds levels of expression in normal or untransformed (nontransgenic) cells or organisms.

“Antisense inhibition” refers to the production of antisense RNA transcripts capable of suppressing the expression of protein from an endogenous gene or a transgene.

“Co-suppression” and “transwitch” each refer to the production of sense RNA transcripts capable of suppressing the expression of identical or substantially similar transgene or endogenous genes (U.S. Pat. No. 5,231,020).

“Gene silencing” refers to homology-dependent suppression of viral genes, transgenes, or endogenous nuclear genes. Gene silencing may be transcriptional, when the suppression is due to decreased transcription of the affected genes, or post-transcriptional, when the suppression is due to increased turnover (degradation) of RNA species homologous to the affected genes (English et al., 1996). Gene silencing includes virus-induced gene silencing (Ruiz et al. 1998).

“Silencing suppressor” gene refers to a gene whose expression leads to counteracting gene silencing and enhanced expression of silenced genes. Silencing suppressor genes may be of plant, non-plant, or viral origin. Examples include, but are not limited to HC-Pro, P1-HC-Pro, and 2b proteins. Other examples include one or more genes in TGMV-B genome.

The terms “heterologous DNA sequence,” “exogenous DNA segment” or “heterologous nucleic acid,” as used herein, each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

“Homologous to” in the context of nucleotide sequence identity refers to the similarity between the nucleotide sequence of two nucleic acid molecules or between the amino acid sequences of two protein molecules. Estimates of such homology are provided by either DNA-DNA or DNA-RNA hybridization under conditions of stringency as is well understood by those skilled in the art (as described in Haines and Higgins (eds.), Nucleic Acid Hybridization, IRL Press, Oxford, U.K.), or by the comparison of sequence similarity between two nucleic acids or proteins.

The term “substantially similar” refers to nucleotide and amino acid sequences that represent functional and/or structural equivalents of Arabidopsis sequences disclosed herein. For example, altered nucleotide sequences which simply reflect the degeneracy of the genetic code but nonetheless encode amino acid sequences that are identical to a particular amino acid sequence are substantially similar to the particular sequences. In addition, amino acid sequences that are substantially similar to a particular sequence are those wherein overall amino acid identity is at least 65% or greater to the instant sequences. Modifications that result in equivalent nucleotide or amino acid sequences are well within the routine skill in the art. Moreover, the skilled artisan recognizes that equivalent nucleotide sequences encompassed by this invention can also be defined by their ability to hybridize, under low, moderate and/or stringent conditions (e.g., 0.1×SSC, 0.1% SDS, 65° C.), with the nucleotide sequences that are within the literal scope of the instant claims.

“Target gene” refers to a gene on the replicon that expresses the desired target coding sequence, functional RNA, or protein. The target gene is not essential for replicon replication. Additionally, target genes may comprise native non-viral genes inserted into a non-native organism, or chimeric genes, and will be under the control of suitable regulatory sequences. Thus, the regulatory sequences in the target gene may come from any source, including the virus. Target genes may include coding sequences that are either heterologous or homologous to the genes of a particular plant to be transformed. However, target genes do not include native viral genes. Typical target genes include, but are not limited to genes encoding a structural protein, a seed storage protein, a protein that conveys herbicide resistance, and a protein that conveys insect resistance. Proteins encoded by target genes are known as “foreign proteins”. The expression of a target gene in a plant will typically produce an altered plant trait.

The term “altered plant trait” means any phenotypic or genotypic change in a transgenic plant relative to the wild-type or non-transgenic plant host.

“Transcription Stop Fragment” refers to nucleotide sequences that contain one or more regulatory signals, such as polyadenylation signal sequences, capable of terminating transcription. Examples include the 3′ non-regulatory regions of genes encoding nopaline synthase and the small subunit of ribulose bisphosphate carboxylase.

“Replication gene” refers to a gene encoding a viral replication protein. In addition to the ORF of the replication protein, the replication gene may also contain other overlapping or non-overlapping ORF(s), as are found in viral sequences in nature. While not essential for replication, these additional ORFs may enhance replication and/or viral DNA accumulation. Examples of such additional ORFs are AC3 and AL3 in ACMV and TGMV geminiviruses, respectively.

“Chimeric trans-acting replication gene” refers either to a replication gene in which the coding sequence of a replication protein is under the control of a regulated plant promoter other than that in the native viral replication gene, or a modified native viral replication gene, for example, in which a site specific sequence(s) is inserted in the 5′ transcribed but untranslated region. Such chimeric genes also include insertion of the known sites of replication protein binding between the promoter and the transcription start site that attenuate transcription of viral replication protein gene.

“Chromosomally-integrated” refers to the integration of a foreign gene or DNA construct into the host DNA by covalent bonds. Where genes are not “chromosomally integrated” they may be “transiently expressed.” Transient expression of a gene refers to the expression of a gene that is not integrated into the host chromosome but functions independently, either as part of an autonomously replicating plasmid or expression cassette, for example, or as part of another biological system such as a virus.

“Production tissue” refers to mature, harvestable tissue consisting of non-dividing, terminally-differentiated cells. It excludes young, growing tissue consisting of germline, meristematic, and not-fully-differentiated cells.

“Germline cells” refer to cells that are destined to be gametes and whose genetic material is heritable.

“Trans-activation” refers to switching on of gene expression or replicon replication by the expression of another (regulatory) gene in trans.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”. Examples of methods of transformation of plants and plant cells include Agrobacterium-mediated transformation (De Blaere et al., 1987) and particle bombardment technology (Klein et al. 1987; U.S. Pat. No. 4,945,050). Whole plants may be regenerated from transgenic cells by methods well known to the skilled artisan (see, for example, Fromm et al., 1990).

“Transformed,” “transgenic,” and “recombinant” refer to a host organism such as a bacterium or a plant into which a heterologous nucleic acid molecule has been introduced. The nucleic acid molecule can be stably integrated into the genome generally known in the art and are disclosed in Sambrook et al., 1989. See also Innis et al., 1995 and Gelfand, 1995; and Innis and Gelfand, 1999. Known methods of PCR include, but are not limited to, methods using paired primers, nested primers, single specific primers, degenerate primers, gene-specific primers, vector-specific primers, partially mismatched primers, and the like. For example, “transformed,” “transformant,” and “transgenic” plants or calli have been through the transformation process and contain a foreign gene integrated into their chromosome. The term “untransformed” refers to normal plants that have not been through the transformation process.

“Transiently transformed” refers to cells in which transgenes and foreign DNA have been introduced (for example, by such methods as Agrobacterium-mediated transformation or biolistic bombardment), but not selected for stable maintenance.

“Stably transformed” refers to cells that have been selected and regenerated on a selection media following transformation.

“Transient expression” refers to expression in cells in which a virus or a transgene is introduced by viral infection or by such methods as Agrobacterium-mediated transformation, electroporation, or biolistic bombardment, but not selected for its stable maintenance.

“Genetically stable” and “heritable” refer to chromosomally-integrated genetic elements that are stably maintained in the plant and stably inherited by progeny through successive generations.

“Primary transformant” and “T0 generation” refer to transgenic plants that are of the same genetic generation as the tissue which was initially transformed (i.e., not having gone through meiosis and fertilization since transformation).

“Secondary transformants” and the “T1, T2, T3, etc. generations” refer to transgenic plants derived from primary transformants through one or more meiotic and fertilization cycles. They may be derived by self-fertilization of primary or secondary transformants or crosses of primary or secondary transformants with other transformed or untransformed plants.

“Wild-type” refers to a virus or organism found in nature without any known mutation.

“Genome” refers to the complete genetic material of an organism.

The term “nucleic acid” refers to deoxyribonucleotides or ribonucleotides and polymers thereof in either single- or double-stranded form, composed of monomers (nucleotides) containing a sugar, phosphate and a base which is either a purine or pyrimidine. Unless specifically limited, the term encompasses nucleic acids containing known analogs of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., 1991; Ohtsuka et al., 1985; Rossolini et al. 1994). A “nucleic acid fragment” is a fraction of a given nucleic acid molecule. In higher plants, deoxyribonucleic acid (DNA) is the genetic material while ribonucleic acid (RNA) is involved in the transfer of information contained within DNA into proteins. The term “nucleotide sequence” refers to a polymer of DNA or RNA which can be single- or double-stranded, optionally containing synthetic, non-natural or altered nucleotide bases capable of incorporation into DNA or RNA polymers. The terms “nucleic acid” or “nucleic acid sequence” may also be used interchangeably with gene, cDNA, DNA and RNA encoded by a gene.

The invention encompasses isolated or substantially purified nucleic acid or protein compositions. In the context of the present invention, an “isolated” or “purified” DNA molecule or an “isolated” or “purified” polypeptide is a DNA molecule or polypeptide that, by the hand of man, exists apart from its native environment and is therefore not a product of nature. An isolated DNA molecule or polypeptide may exist in a purified form or may exist in a non-native environment such as, for example, a transgenic host cell. For example, an “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. Preferably, an “isolated” nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein or polypeptide having less than about 30%, 20%, 10%, 5%, (by dry weight) of contaminating protein. When the protein of the invention, or biologically active portion thereof, is recombinantly produced, preferably culture medium represents less than about 30%, 20%, 10%, or 5% (by dry weight) of chemical precursors or non-protein of interest chemicals.

The nucleotide sequences of the invention include both the naturally occurring sequences as well as mutant (variant) forms. Such variants will continue to possess the desired activity, i.e., either promoter activity or the activity of the product encoded by the open reading frame of the non-variant nucleotide sequence.

Thus, by “variants” is intended substantially similar sequences. For nucleotide sequences comprising an open reading frame, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis and for open reading frames, encode the native protein, as well as those that encode a polypeptide having amino acid substitutions relative to the native protein. Generally, nucleotide sequence variants of the invention will have at least 40, 50, 60, to 70%, e.g., preferably 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98% and 99% nucleotide sequence identity to the native (wild type or endogenous) nucleotide sequence.

“Conservatively modified variations” of a particular nucleic acid sequence refers to those nucleic acid sequences that encode identical or essentially identical amino acid sequences, or where the nucleic acid sequence does not encode an amino acid sequence, to essentially identical sequences. Because of the degeneracy of the genetic code, a large number of functionally identical nucleic acids encode any given polypeptide. For instance the codons CGT, CGC, CGA, CGG, AGA, and AGG all encode the amino acid arginine. Thus, at every position where an arginine is specified by a codon, the codon can be altered to any of the corresponding codons described without altering the encoded protein. Such nucleic acid variations are “silent variations” which are one species of “conservatively modified variations.” Every nucleic acid sequence described herein which encodes a polypeptide also describes every possible silent variation, except where otherwise noted. One of skill will recognize that each codon in a nucleic acid (except ATG, which is ordinarily the only codon for methionine) can be modified to yield a functionally identical molecule by standard techniques. Accordingly, each “silent variation” of a nucleic acid which encodes a polypeptide is implicit in each described sequence.

The nucleic acid molecules of the invention can be “optimized” for enhanced expression in plants of interest. See, for example, EPA 035472; WO 91/16432; Perlak et al., 1991; and Murray et al., 1989. In this manner, the open reading frames in genes or gene fragments can be synthesized utilizing plant-preferred codons. See, for example, Campbell and Gowri, 1990 for a discussion of host-preferred codon usage. Thus, the nucleotide sequences can be optimized for expression in any plant. It is recognized that all or any part of the gene sequence may be optimized or synthetic. That is, synthetic or partially optimized sequences may also be used. Variant nucleotide sequences and proteins also encompass sequences and protein derived from a mutagenic and recombinogenic procedure such as DNA shuffling. With such a procedure, one or more different coding sequences can be manipulated to create a new polypeptide possessing the desired properties. In this manner, libraries of recombinant polynucleotides are generated from a population of related sequence polynucleotides comprising sequence regions that have substantial sequence identity and can be homologously recombined in vitro or in vivo. Strategies for such DNA shuffling are known in the art. See, for example, Stemmer, 1994; Stemmer, 1994; Crameri et al., 1997; Moore et al., 1997; Zhang et al., 1997; Crameri et al., 1998; and U.S. Pat. Nos. 5,605,793 and 5,837,458.

By “variant” polypeptide is intended a polypeptide derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Such variants may result from, for example, genetic polymorphism or from human manipulation. Methods for such manipulations are generally known in the art.

Thus, the polypeptides may be altered in various ways including amino acid substitutions, deletions, truncations, and insertions. Methods for such manipulations are generally known in the art. For example, amino acid sequence variants of the polypeptides can be prepared by mutations in the DNA. Methods for mutagenesis and nucleotide sequence alterations are well known in the art. See, for example, Kunkel, 1985; Kunkel et al., 1987; U.S. Pat. No. 4,873,192; Walker and Gaastra, 1983 and the references cited therein. Guidance as to appropriate amino acid substitutions that do not affect biological activity of the protein of interest may be found in the model of Dayhoff et al. (1978). Conservative substitutions, such as exchanging one amino acid with another having similar properties, are preferred.

Individual substitutions deletions or additions that alter, add or delete a single amino acid or a small percentage of amino acids (typically less than 5%, more typically less than 1%) in an encoded sequence are “conservatively modified variations,” where the alterations result in the substitution of an amino acid with a chemically similar amino acid. Conservative substitution tables providing functionally similar amino acids are well known in the art. The following five groups each contain amino acids that are conservative substitutions for one another: Aliphatic: Glycine (G), Alanine (A), Valine (V), Leucine (L), Isoleucine (I); Aromatic: Phenylalanine (F), Tyrosine (Y), Tryptophan (W); Sulfur-containing: Methionine (M), Cysteine (C); Basic: Arginine I, Lysine (K), Histidine (H); Acidic: Aspartic acid (D), Glutamic acid (E), Asparagine (N), Glutamine (Q). See also, Creighton, 1984. In addition, individual substitutions, deletions or additions which alter, add or delete a single amino acid or a small percentage of amino acids in an encoded sequence are also “conservatively modified variations.”

“Expression cassette” as used herein means a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one which is naturally occurring but has been obtained in a recombinant form useful for heterologous expression. The expression of the nucleotide sequence in the expression cassette may be under the control of a constitutive promoter or of an inducible promoter which initiates transcription only when the host cell is exposed to some particular external stimulus. In the case of a multicellular organism, the promoter can also be specific to a particular tissue or organ or stage of development.

“Vector” is defined to include, inter alia, any plasmid, cosmid, phage or Agrobacterium binary vector in double or single stranded linear or circular form which may or may not be self transmissible or mobilizable, and which can transform prokaryotic or eukaryotic host either by integration into the cellular genome or exist extrachromosomally (e.g. autonomous replicating plasmid with an origin of replication).

Specifically included are shuttle vectors by which is meant a DNA vehicle capable, naturally or by design, of replication in two different host organisms, which may be selected from actinomycetes and related species, bacteria and eukaryotic (e.g. higher plant, mammalian, yeast or fungal cells).

Preferably the nucleic acid in the vector is under the control of, and operably linked to, an appropriate promoter or other regulatory elements for transcription in a host cell such as a microbial, e.g. bacterial, or plant cell. The vector may be a bi-functional expression vector which functions in multiple hosts. In the case of genomic DNA, this may contain its own promoter or other regulatory elements and in the case of cDNA this may be under the control of an appropriate promoter or other regulatory elements for expression in the host cell.

“Cloning vectors” typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance or ampicillin resistance.

A “transgenic plant” is a plant having one or more plant cells that contain an expression vector.

“Plant tissue” includes differentiated and undifferentiated tissues or plants, including but not limited to roots, stems, shoots, leaves, pollen, seeds, tumor tissue and various forms of cells and culture such as single cells, protoplast, embryos, and callus tissue. The plant tissue may be in plants or in organ, tissue or cell culture.

The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”.

(a) As used herein, “reference sequence” is a defined sequence used as a basis for sequence comparison. A reference sequence may be a subset or the entirety of a specified sequence; for example, as a segment of a full length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent identity between any two sequences can be accomplished using a mathematical algorithm. Preferred, non-limiting examples of such mathematical algorithms are the algorithm of Myers and Miller, 1988; the local homology algorithm of Smith et al. 1981; the homology alignment algorithm of Needleman and Wunsch 1970; the search-for-similarity-method of Pearson and Lipman 1988; the algorithm of Karlin and Altschul, 1990, modified as in Karlin and Altschul, 1993.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. 1988; Higgins et al. 1989; Corpet et al. 1988; Huang et al. 1992; and Pearson et al. 1994. The ALIGN program is based on the algorithm of Myers and Miller, supra. The BLAST programs of Altschul et al., 1990, are based on the algorithm of Karlin and Altschul supra.

Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences (see, e.g., Karlin & Altschul (1993). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

To obtain gapped alignments for comparison purposes, Gapped BLAST (in BLAST 2.0) can be utilized as described in Altschul et al. 1997. Alternatively, PSI-BLAST (in BLAST 2.0) can be used to perform an iterated search that detects distant relationships between molecules. See Altschul et al., supra. When utilizing BLAST, Gapped BLAST, PSI-BLAST, the default parameters of the respective programs (e.g. BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, 1989). See http://www.ncbi.nlm.nih.gov. Alignment may also be performed manually by inspection.

For purposes of the present invention, comparison of nucleotide sequences for determination of percent sequence identity to the promoter sequences disclosed herein is preferably made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.

(c) As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70%, 71%, 72%, 73%, 74%, 75%, 76%, 77%, 78%, or 79%, preferably at least 80%, 81%, 82%, 83%, 84%, 85%, 86%, 87%, 88%, or 89%, more preferably at least 90%, 91%, 92%, 93%, or 94%, and most preferably at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, more preferably at least 80%, 90%, and most preferably at least 95%.

Another indication that nucleotide sequences are substantially identical is if two molecules hybridize to each other under stringent conditions (see below). Generally, stringent conditions are selected to be about 5° C. lower than the thermal melting point (Tₘ) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 70%, 71%, 72%, 73%, 74%, 75%, 76%, 77%, 78%, or 79%, preferably 80%, 81%, 82%, 83%, 84%, 85%, 86%, 87%, 88%, or 89%, more preferably at least 90%, 91%, 92%, 93%, or 94%, or even more preferably, 95%, 96%, 97%, 98% or 99%, sequence identity to the reference sequence over a specified comparison window. Preferably, optimal alignment is conducted using the homology alignment algorithm of Needleman and Wunsch (1970). An indication that two peptide sequences are substantially identical is that one peptide is immunologically reactive with antibodies raised against the second peptide. Thus, a peptide is substantially identical to a second peptide, for example, where the two peptides differ only by a conservative substitution.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

As noted above, another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridization are sequence dependent, and are different under different environmental parameters. The Tₘ is the temperature (under defined ionic strength and pH) at which 50% of the target sequence hybridizes to a perfectly matched probe. Specificity is typically the function of post-hybridization washes, the critical factors being the ionic strength and temperature of the final wash solution. For DNA-DNA hybrids, the Tₘ can be approximated from the equation of Meinkoth and Wahl, 1984; Tₘ 81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the thermal melting point I for the specific sequence and its complement at a defined ionic strength and pH. However, severely stringent conditions can utilize a hybridization and/or wash at 1, 2, 3, or 4° C. lower than the thermal melting point I; moderately stringent conditions can utilize a hybridization and/or wash at 6, 7, 8, 9, or 10° C. lower than the thermal melting point I; low stringency conditions can utilize a hybridization and/or wash at 11, 12, 13, 14, 15, or 20° C. lower than the thermal melting point I. Using the equation, hybridization and wash compositions, and desired T, those of ordinary skill will understand that variations in the stringency of hybridization and/or wash solutions are inherently described. If the desired degree of mismatching results in a T of less than 45° C. (aqueous solution) or 32° C. (formamide solution), it is preferred to increase the SSC concentration so that a higher temperature can be used. An extensive guide to the hybridization of nucleic acids is found in Tijssen, 1993. Generally, highly stringent hybridization and wash conditions are selected to be about 5° C. lower than the thermal melting point Tₘ for the specific sequence at a defined ionic strength and pH.

An example of highly stringent wash conditions is 0.15 M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.5 M, more preferably about 0.01 to 1.0 M, Na ion concentration (or other salts) at pH 7.0 to 8.3, and the temperature is typically at least about 30° C. and at least about 60° C. for long robes (e.g., >50 nucleotides). Stringent conditions may also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

Very stringent conditions are selected to be equal to the Tₘ for a particular probe. An example of stringent conditions for hybridization of complementary nucleic acids which have more than 100 complementary residues on a filter in a Southern or Northern blot is 50% formamide, e.g., hybridization in 50% formamide, 1 M NaCl, 1% SDS at 37° C., and a wash in 0.1×SSC at 60 to 65° C. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C.

The following are examples of sets of hybridization/wash conditions that may be used to clone orthologous nucleotide sequences that are substantially identical to reference nucleotide sequences of the present invention: a reference nucleotide sequence preferably hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, 1 mM EDTA at 50° C. with washing in 1×SSC, 0.1% SDS at 50° C., more desirably still in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5 M NaPO₄, 1 mM EDTA at 50° C. with washing in 0.1×SSC, 0.1% SDS at 65° C.

“DNA shuffling” is a method to introduce mutations or rearrangements, preferably randomly, in a DNA molecule or to generate exchanges of DNA sequences between two or more DNA molecules, preferably randomly. The DNA molecule resulting from DNA shuffling is a shuffled DNA molecule that is a non-naturally occurring DNA molecule derived from at least one template DNA molecule. The shuffled DNA preferably encodes a variant polypeptide modified with respect to the polypeptide encoded by the template DNA, and may have an altered biological activity with respect to the polypeptide encoded by the template DNA.

“Recombinant DNA molecule” is a combination of DNA sequences that are joined together using recombinant DNA technology and procedures used to join together DNA sequences as described, for example, in Sambrook et al., 1989.

The word “plant” refers to any plant, particularly to seed plant, and “plant cell” is a structural and physiological unit of the plant, which comprises a cell wall but may also refer to a protoplast. The plant cell may be in form of an isolated single cell or a cultured cell, or as a part of higher organized unit such as, for example, a plant tissue, or a plant organ.

“Significant increase” is an increase that is larger than the margin of error inherent in the measurement technique, preferably an increase by about 2-fold or greater.

“Significantly less” means that the decrease is larger than the margin of error inherent in the measurement technique, preferably a decrease by about 2-fold or greater.

II. DNA Sequences for Transformation

Virtually any DNA composition may be used for delivery to recipient plant cells, e.g., monocotyledonous cells, to ultimately produce fertile transgenic plants in accordance with the present invention. For example, DNA segments in the form of vectors and plasmids, or linear DNA fragments, in some instances containing only the DNA element to be expressed in the plant, and the like, may be employed. The construction of vectors which may be employed in conjunction with the present invention will be known to those of skill of the art in light of the present disclosure (see, e.g., Sambrook et al., 1989; Gelvin et al., 1990).

Vectors, plasmids, cosmids, YACs (yeast artificial chromosomes), BACs (bacterial artificial chromosomes) and DNA segments for use in transforming such cells will, of course, generally comprise the cDNA, gene or genes which one desires to introduce into the cells. These DNA constructs can further include structures such as promoters, enhancers, polylinkers, or even regulatory genes as desired. The DNA segment or gene chosen for cellular introduction will often encode a protein which will be expressed in the resultant recombinant cells, such as will result in a screenable or selectable trait and/or which will impart an improved phenotype to the regenerated plant. However, this may not always be the case, and the present invention also encompasses transgenic plants incorporating non-expressed transgenes.

In certain embodiments, it is contemplated that one may wish to employ replication-competent viral vectors in monocot transformation. Such vectors include, for example, wheat dwarf virus (WDV) “shuttle” vectors, such as pW1-11 and PW1-GUS (Ugaki et al., 1991). These vectors are capable of autonomous replication in maize cells as well as E. coli, and as such may provide increased sensitivity for detecting DNA delivered to transgenic cells. A replicating vector may also be useful for delivery of genes flanked by DNA sequences from transposable elements such as Ac, Ds, or Mu. It has been proposed (Laufs et al., 1990) that transposition of these elements within the maize genome requires DNA replication. It is also contemplated that transposable elements would be useful for introducing DNA fragments lacking elements necessary for selection and maintenance of the plasmid vector in bacteria, e.g., antibiotic resistance genes and origins of DNA replication. It is also proposed that use of a transposable element such as Ac, Ds, or Mu would actively promote integration of the desired DNA and hence increase the frequency of stably transformed cells. The use of a transposable element such as Ac, Ds, or Mu may actively promote integration of the DNA of interest and hence increase the frequency of stably transformed cells. Transposable elements may be useful to allow separation of genes of interest from elements necessary for selection and maintenance of a plasmid vector in bacteria or selection of a transformant. By use of a transposable element, desirable and undesirable DNA sequences may be transposed apart from each other in the genome, such that through genetic segregation in progeny, one may identify plants with either the desirable or the undesirable DNA sequences.

DNA useful for introduction into plant cells includes that which has been derived or isolated from any source, that may be subsequently characterized as to structure, size and/or function, chemically altered, and later introduced into plants. An example of DNA “derived” from a source, would be a DNA sequence that is identified as a useful fragment within a given organism, and which is then chemically synthesized in essentially pure form. An example of such DNA “isolated” from a source would be a useful DNA sequence that is excised or removed from said source by chemical means, e.g., by the use of restriction endonucleases, so that it can be further manipulated, e.g., amplified, for use in the invention, by the methodology of genetic engineering. Such DNA is commonly referred to as “recombinant DNA.”

Therefore useful DNA includes completely synthetic DNA, semi-synthetic DNA, DNA isolated from biological sources, and DNA derived from introduced RNA. Generally, the introduced DNA is not originally resident in the plant genotype which is the recipient of the DNA, but it is within the scope of the invention to isolate a gene from a given plant genotype, and to subsequently introduce multiple copies of the gene into the same genotype, e.g., to enhance production of a given gene product such as a storage protein or a protein that confers tolerance or resistance to water deficit.

The introduced DNA includes but is not limited to, DNA from plant genes, and non-plant genes such as those from bacteria, yeasts, animals or viruses. The introduced DNA can include modified genes, portions of genes, or chimeric genes, including genes from the same or different maize genotype. The term “chimeric gene” or “chimeric DNA” is defined as a gene or DNA sequence or segment comprising at least two DNA sequences or segments from species which do not combine DNA under natural conditions, or which DNA sequences or segments are positioned or linked in a manner which does not normally occur in the native genome of untransformed plant.

The introduced DNA used for transformation herein may be circular or linear, double-stranded or single-stranded. Generally, the DNA is in the form of chimeric DNA, such as plasmid DNA, that can also contain coding regions flanked by regulatory sequences which promote the expression of the recombinant DNA present in the resultant plant. For example, the DNA may itself comprise or consist of a promoter that is active in a plant which is derived from a source other than that plant, or may utilize a promoter already present in a plant genotype that is the transformation target.

Generally, the introduced DNA will be relatively small, i.e., less than about 30 kb to minimize any susceptibility to physical, chemical, or enzymatic degradation which is known to increase as the size of the DNA increases. As noted above, the number of proteins, RNA transcripts or mixtures thereof which is introduced into the plant genome is preferably preselected and defined, e.g., from one to about 5-10 such products of the introduced DNA may be formed.

Two principal methods for the control of expression are known, viz.: overexpression and underexpression. Overexpression can be achieved by insertion of one or more than one extra copy of the selected gene. It is, however, not unknown for plants or their progeny, originally transformed with one or more than one extra copy of a nucleotide sequence, to exhibit the effects of underexpression as well as overexpression. For underexpression there are two principle methods which are commonly referred to in the art as “antisense downregulation” and “sense downregulation” (sense downregulation is also referred to as “cosuppression”). Generically these processes are referred to as “gene silencing”. Both of these methods lead to an inhibition of expression of the target gene.

Obtaining sufficient levels of transgene expression in the appropriate plant tissues is an important aspect in the production of genetically engineered crops. Expression of heterologous DNA sequences in a plant host is dependent upon the presence of an operably linked promoter that is functional within the plant host. Choice of the promoter sequence will determine when and where within the organism the heterologous DNA sequence is expressed.

Furthermore, it is contemplated that promoters combining elements from more than one promoter may be useful. For example, U.S. Pat. No. 5,491,288 discloses combining a Cauliflower Mosaic Virus promoter with a histone promoter. Thus, the elements from the promoters disclosed herein may be combined with elements from other promoters.

Promoters which are useful for plant transgene expression include those that are inducible, viral, synthetic, constitutive (Odell et al., 1985), temporally regulated, spatially regulated, tissue-specific, and spatio-temporally regulated.

Where expression in specific tissues or organs is desired, tissue-specific promoters may be used. In contrast, where gene expression in response to a stimulus is desired, inducible promoters are the regulatory elements of choice. Where continuous expression is desired throughout the cells of a plant, constitutive promoters are utilized. Additional regulatory sequences upstream and/or downstream from the core promoter sequence may be included in expression constructs of transformation vectors to bring about varying levels of expression of heterologous nucleotide sequences in a transgenic plant.

A. Transcription Regulatory Sequences

1. Promoters

The choice of promoter will vary depending on the temporal and spatial requirements for expression, and also depending on the target species. In some cases, expression in multiple tissues is desirable. While in others, tissue-specific, e.g., leaf-specific, seed-specific, petal-specific, anther-specific, or pith-specific, expression is desirable. Although many promoters from dicotyledons have been shown to be operational in monocotyledons and vice versa, ideally dicotyledonous promoters are selected for expression in dicotyledons, and monocotyledonous promoters for expression in monocotyledons. However, there is no restriction to the provenance of selected promoters; it is sufficient that they are operational in driving the expression of the nucleotide sequences in the desired cell.

These promoters include, but are not limited to, constitutive, inducible, temporally regulated, developmentally regulated, spatially-regulated, chemically regulated, stress-responsive, tissue-specific, viral and synthetic promoters. Promoter sequences are known to be strong or weak. A strong promoter provides for a high level of gene expression, whereas a weak promoter provides for a very low level of gene expression. An inducible promoter is a promoter that provides for the turning on and off of gene expression in response to an exogenously added agent, or to an environmental or developmental stimulus. A bacterial promoter such as the P_{tac} promoter can be induced to varying levels of gene expression depending on the level of isothiopropylgalactoside added to the transformed bacterial cells. An isolated promoter sequence that is a strong promoter for heterologous nucleic acid is advantageous because it provides for a sufficient level of gene expression to allow for easy detection and selection of transformed cells and provides for a high level of gene expression when desired.

Within a plant promoter region there are several domains that are necessary for full function of the promoter. The first of these domains lies immediately upstream of the structural gene and forms the “core promoter region” containing consensus sequences, normally 70 base pairs immediately upstream of the gene. The core promoter region contains the characteristic CAAT and TATA boxes plus surrounding sequences, and represents a transcription initiation sequence that defines the transcription start point for the structural gene.

The presence of the core promoter region defines a sequence as being a promoter: if the region is absent, the promoter is non-functional. Furthermore, the core promoter region is insufficient to provide fall promoter activity. A series of regulatory sequences upstream of the core constitute the remainder of the promoter. The regulatory sequences determine expression level, the spatial and temporal pattern of expression and, for an important subset of promoters, expression under inductive conditions (regulation by external factors such as light, temperature, chemicals, hormones).

A range of naturally-occurring promoters are known to be operative in plants and have been used to drive the expression of heterologous (both foreign and endogenous) genes in plants: for example, the constitutive 35S cauliflower mosaic virus (CaMV) promoter, the ripening-enhanced tomato polygalacturonase promoter (Bird et al., 1988), the E8 promoter (Diekman & Fischer, 1988) and the fruit specific 2A1 promoter (Pear et al., 1989) and many others, e.g., U2 and U5 snRNA promoters from maize, the promoter from alcohol dehydrogenase, the Z4 promoter from a gene encoding the Z4 22 kD zein protein, the Z10 promoter from a gene encoding a 10 kD zein protein, a Z27 promoter from a gene encoding a 27 kD zein protein, the A20 promoter from the gene encoding a 19 kD-zein protein, inducible promoters, such as the light inducible promoter derived from the pea rbcS gene and the actin promoter from rice, e.g., the actin 2 promoter (WO 00/70067); seed specific promoters, such as the phaseolin promoter from beans, may also be used. The nucleotide sequences of this invention can also be expressed under the regulation of promoters that are chemically regulated. This enables the nucleic acid sequence or encoded polypeptide to be synthesized only when the crop plants are treated with the inducing chemicals. Chemical induction of gene expression is detailed in EP 0 332 104 (to Ciba-Geigy) and U.S. Pat. No. 5,614,395. A preferred promoter for chemical induction is the tobacco PR-1a promoter.

Examples of some constitutive promoters which have been described include the rice actin 1 (Wang et al., 1992; U.S. Pat. No. 5,641,876), CaMV 35S (Odell et al., 1985), CaMV 19S (Lawton et al., 1987), nos, Adh, sucrose synthase; and the ubiquitin promoters.

Examples of tissue specific promoters which have been described include the lectin (Vodkin, 1983; Lindstrom et al., 1990) corn alcohol dehydrogenase 1 (Vogel et al., 1989; Dennis et al., 1984), corn light harvesting complex (Simpson, 1986; Bansal et al., 1992), corn heat shock protein (Odell et al., 1985), pea small subunit RuBP carboxylase (Poulsen et al., 1986), Ti plasmid mannopine synthase (Langridge et al., 1989), Ti plasmid nopaline synthase (Langridge et al., 1989), petunia chalcone isomerase (vanTunen et al., 1988), bean glycine rich protein 1 (Keller et al., 1989), truncated CaMV 35S (Odell et al., 1985), potato patatin (Wenzler et al., 1989), root cell (Yamamoto et al., 1990), maize zein (Reina et al., 1990; Kriz et al., 1987; Wandelt et al., 1989; Langridge et al., 1983; Reina et al., 1990), globulin-1 (Belanger et al., 1991), α-tubulin, cab (Sullivan et al., 1989), PEPCase (Hudspeth & Grula, 1989), R gene complex-associated promoters (Chandler et al., 1989), histone, and chalcone synthase promoters (Franken et al., 1991). Tissue specific enhancers are described in Fromm et al. (1989).

Inducible promoters that have been described include the ABA- and turgor-inducible promoters, the promoter of the auxin-binding protein gene (Schwob et al., 1993), the UDP glucose flavonoid glycosyl-transferase gene promoter (Ralston et al., 1988), the MPI proteinase inhibitor promoter (Cordero et al., 1994), and the glyceraldehyde-3-phosphate dehydrogenase gene promoter (Kohler et al., 1995; Quigley et al., 1989; Martinez et al., 1989).

Several other tissue-specific regulated genes and/or promoters have been reported in plants. These include genes encoding the seed storage proteins (such as napin, cruciferin, beta-conglycinin, and phaseolin) zein or oil body proteins (such as oleosin), or genes involved in fatty acid biosynthesis (including acyl carrier protein, stearoyl-ACP desaturase. And fatty acid desaturases (fad 2-1)), and other genes expressed during embryo development (such as Bce4, see, for example, EP 255378 and Kridl et al., 1991). Particularly useful for seed-specific expression is the pea vicilin promoter (Czako et al., 1992). (See also U.S. Pat. No. 5,625,136, herein incorporated by reference.) Other useful promoters for expression in mature leaves are those that are switched on at the onset of senescence, such as the SAG promoter from Arabidopsis (Gan et al., 1995).

A class of fruit-specific promoters expressed at or during antithesis through fruit development, at least until the beginning of ripening, is discussed in U.S. Pat. No. 4,943,674. cDNA clones that are preferentially expressed in cotton fiber have been isolated (John et al., 1992). cDNA clones from tomato displaying differential expression during fruit development have been isolated and characterized (Mansson et al., 1985, Slater et al., 1985). The promoter for polygalacturonase gene is active in fruit ripening. The polygalacturonase gene is described in U.S. Pat. No. 4,535,060, U.S. Pat. No. 4,769,061, U.S. Pat. No. 4,801,590, and U.S. Pat. No. 5,107,065, which disclosures are incorporated herein by reference.

Other examples of tissue-specific promoters include those that direct expression in leaf cells following damage to the leaf (for example, from chewing insects), in tubers (for example, patatin gene promoter), and in fiber cells (an example of a developmentally-regulated fiber cell protein is E6 (John et al., 1992). The E6 gene is most active in fiber, although low levels of transcripts are found in leaf, ovule and flower.

The tissue-specificity of some “tissue-specific” promoters may not be absolute and may be tested by one skilled in the art using the diphtheria toxin sequence. One can also achieve tissue-specific expression with “leaky” expression by a combination of different tissue-specific promoters (Beals et al., 1997). Other tissue-specific promoters can be isolated by one skilled in the art (see U.S. Pat. No. 5,589,379). Several inducible promoters (“gene switches”) have been reported. Many are described in the review by Gatz (1996) and Gatz (1997). These include tetracycline repressor system, Lac repressor system, copper-inducible systems, salicylate-inducible systems (such as the PR1a system), glucocorticoid- (Aoyama et al., 1997) and ecdysome-inducible systems. Also included are the benzene sulphonamide- (U.S. Pat. No. 5,364,780) and alcohol-(WO 97/06269 and WO 97/06268) inducible systems and glutathione S-transferase promoters. Other studies have focused on genes inducibly regulated in response to environmental stress or stimuli such as increased salinity. Drought, pathogen and wounding. (Graham et al., 1985; Graham et al., 1985, Smith et al., 1986). Accumulation of metallocarboxypeptidase-inhibitor protein has been reported in leaves of wounded potato plants (Graham et al., 1981). Other plant genes have been reported to be induced methyl jasmonate, elicitors, heat-shock, anaerobic stress, or herbicide safeners.

Regulated expression of the chimeric transacting viral replication protein can be further regulated by other genetic strategies. For example, Cre-mediated gene activation as described by Odell et al. 1990. Thus, a DNA fragment containing 3′ regulatory sequence bound by lox sites between the promoter and the replication protein coding sequence that blocks the expression of a chimeric replication gene from the promoter can be removed by Cre-mediated excision and result in the expression of the trans-acting replication gene. In this case, the chimeric Cre gene, the chimeric trans-acting replication gene, or both can be under the control of tissue- and developmental-specific or inducible promoters. An alternate genetic strategy is the use of tRNA suppressor gene. For example, the regulated expression of a tRNA suppressor gene can conditionally control expression of a trans-acting replication protein coding sequence containing an appropriate termination codon as described by Ulmasov et al. 1997. Again, either the chimeric tRNA suppressor gene, the chimeric transacting replication gene, or both can be under the control of tissue- and developmental-specific or inducible promoters.

Frequently it is desirable to have continuous or inducible expression of a DNA sequence throughout the cells of an organism in a tissue-independent manner. For example, increased resistance of a plant to infection by soil- and airborne-pathogens might be accomplished by genetic manipulation of the plant's genome to comprise a continuous promoter operably linked to a heterologous pathogen-resistance gene such that pathogen-resistance proteins are continuously expressed throughout the plant's tissues.

Alternatively, it might be desirable to inhibit expression of a native DNA sequence within a plant's tissues to achieve a desired phenotype. In this case, such inhibition might be accomplished with transformation of the plant to comprise a constitutive, tissue-independent promoter operably linked to an antisense nucleotide sequence, such that constitutive expression of the antisense sequence produces an RNA transcript that interferes with translation of the mRNA of the native DNA sequence.

To define a minimal promoter region, a DNA segment representing the promoter region is removed from the 5′ region of the gene of interest and operably linked to the coding sequence of a marker (reporter) gene by recombinant DNA techniques well known to the art. The reporter gene is operably linked downstream of the promoter, so that transcripts initiating at the promoter proceed through the reporter gene. Reporter genes generally encode proteins which are easily measured, including, but not limited to, chloramphenicol acetyl transferase (CAT), beta-glucuronidase (GUS), green fluorescent protein (GFP), beta-galactosidase (beta-GAL), and luciferase.

The construct containing the reporter gene under the control of the promoter is then introduced into an appropriate cell type by transfection techniques well known to the art. To assay for the reporter protein, cell lysates are prepared and appropriate assays, which are well known in the art, for the reporter protein are performed. For example, if CAT were the reporter gene of choice, the lysates from cells transfected with constructs containing CAT under the control of a promoter under study are mixed with isotopically labeled chloramphenicol and acetyl-coenzyme A (acetyl-CoA). The CAT enzyme transfers the acetyl group from acetyl-CoA to the 2- or 3-position of chloramphenicol. The reaction is monitored by thin-layer chromatography, which separates acetylated chloramphenicol from unreacted material. The reaction products are then visualized by autoradiography.

The level of enzyme activity corresponds to the amount of enzyme that was made, which in turn reveals the level of expression from the promoter of interest. This level of expression can be compared to other promoters to determine the relative strength of the promoter under study. In order to be sure that the level of expression is determined by the promoter, rather than by the stability of the mRNA, the level of the reporter mRNA can be measured directly, such as by Northern blot analysis.

Once activity is detected, mutational and/or deletional analyses may be employed to determine the minimal region and/or sequences required to initiate transcription. Thus, sequences can be deleted at the 5′ end of the promoter region and/or at the 3′ end of the promoter region, and nucleotide substitutions introduced. These constructs are then introduced to cells and their activity determined.

In one embodiment, the promoter may be a gamma zein promoter, an oleosin ole16 promoter, a globulinI promoter, an actin I promoter, an actin cl promoter, a sucrose synthetase promoter, an INOPS promoter, an EXM5 promoter, a globulin2 promoter, a b-32, ADPG-pyrophosphorylase promoter, an LtpI promoter, an Ltp2 promoter, an oleosin ole17 promoter, an oleosin ole18 promoter, an actin 2 promoter, a pollen-specific protein promoter, a pollen-specific pectate lyase promoter, an anther-specific protein promoter (Huffman), an anther-specific gene RTS2 promoter, a pollen-specific gene promoter, a tapeturn-specific gene promoter, tapeturn-specific gene RAB24 promoter, a anthranilate synthase alpha subunit promoter, an alpha zein promoter, an anthranilate synthase beta subunit promoter, a dihydrodipicolinate synthase promoter, a Thi1 promoter, an alcohol dehydrogenase promoter, a cab binding protein promoter, an H3C4 promoter, a RUBISCO SS starch branching enzyme promoter, an ACCase promoter, an actin3 promoter, an actin7 promoter, a regulatory protein GF14-12 promoter, a ribosomal protein L9 promoter, a cellulose biosynthetic enzyme promoter, an S-adenosyl-L-homocysteine hydrolase promoter, a superoxide dismutase promoter, a C-kinase receptor promoter, a phosphoglycerate mutase promoter, a root-specific RCc3 mRNA promoter, a glucose-6 phosphate isomerase promoter, a pyrophosphate-fructose 6-phosphatelphosphotransferase promoter, an ubiquitin promoter, a beta-ketoacyl-ACP synthase promoter, a 33 kDa photosystem 11 promoter, an oxygen evolving protein promoter, a 69 kDa vacuolar ATPase subunit promoter, a metallothionein-like protein promoter, a glyceraldehyde-3-phosphate dehydrogenase promoter, an ABA- and ripening-inducible-like protein promoter, a phenylalanine ammonia lyase promoter, an adenosine triphosphatase S-adenosyl-L-homocysteine hydrolase promoter, an a-tubulin promoter, a cab promoter, a PEPCase promoter, an R gene promoter, a lectin promoter, a light harvesting complex promoter, a heat shock protein promoter, a chalcone synthase promoter, a zein promoter, a globulin-1 promoter, an ABA promoter, an auxin-binding protein promoter, a UDP glucose flavonoid glycosyl-transferase gene promoter, an NTI promoter, an actin promoter, an opaque 2 promoter, a b70 promoter, an oleosin promoter, a CaMV 35S promoter, a CaMV 19S promoter, a histone promoter, a turgor-inducible promoter, a pea small subunit RuBP carboxylase promoter, a Ti plasmid mannopine synthase promoter, Ti plasmid nopaline synthase promoter, a petunia chalcone isomerase promoter, a bean glycine rich protein I promoter, a CaMV 35S transcript promoter, a potato patatin promoter, or a S-E9 small subunit RuBP carboxylase promoter.

2. Other Regulatory Elements

In addition to promoters, a variety of 5′ and 3′ transcriptional regulatory sequences are also available for use in the present invention. Transcriptional terminators are responsible for the termination of transcription and correct mRNA polyadenylation. The 3′ nontranslated regulatory DNA sequence preferably includes from about 50 to about 1,000, more preferably about 100 to about 1,000, nucleotide base pairs and contains plant transcriptional and translational termination sequences. Appropriate transcriptional terminators and those which are known to function in plants include the CaMV 35S terminator, the tml terminator, the nopaline synthase terminator, the pea rbcS E9 terminator, the terminator for the T7 transcript from the octopine synthase gene of Agrobacterium tumefaciens, and the 3′ end of the protease inhibitor I or II genes from potato or tomato, although other 3′ elements known to those of skill in the art can also be employed. Alternatively, one also could use a gamma coixin, oleosin 3 or other terminator from the genus Coix.

Preferred 3′ elements include those from the nopaline synthase gene of Agrobacterium tumefaciens (Bevan et al., 1983), the terminator for the T7 transcript from the octopine synthase gene of Agrobacterium tumefaciens, and the 3′ end of the protease inhibitor I or II genes from potato or tomato.

As the DNA sequence between the transcription initiation site and the start of the coding sequence, i.e., the untranslated leader sequence, can influence gene expression, one may also wish to employ a particular leader sequence. Preferred leader sequences are contemplated to include those which include sequences predicted to direct optimum expression of the attached gene, i.e., to include a preferred consensus leader sequence which may increase or maintain mRNA stability and prevent inappropriate initiation of translation. The choice of such sequences will be known to those of skill in the art in light of the present disclosure. Sequences that are derived from genes that are highly expressed in plants will be most preferred.

Other sequences that have been found to enhance gene expression in transgenic plants include intron sequences (e.g., from Adh1, bronze1, actin1, actin 2 (WO 00/760067), or the sucrose synthase intron) and viral leader sequences (e.g., from TMV, MCMV and AMV). For example, a number of non-translated leader sequences derived from viruses are known to enhance expression. Specifically, leader sequences from Tobacco Mosaic Virus (TMV), Maize Chlorotic Mottle Virus (MCMV), and Alfalfa Mosaic Virus (AMV) have been shown to be effective in enhancing expression (e.g., Gallie et al., 1987; Skuzeski et al., 1990). Other leaders known in the art include but are not limited to: Picornavirus leaders, for example, EMCV leader (Encephalomyocarditis 5 noncoding region) (Elroy-Stein et al., 1989); Potyvirus leaders, for example, TEV leader (Tobacco Etch Virus); MDMV leader (Maize Dwarf Mosaic Virus); Human immunoglobulin heavy-chain binding protein (BiP) leader, (Macejak et al., 1991); Untranslated leader from the coat protein mRNA of alfalfa mosaic virus (AMV RNA 4), (Jobling et al., 1987; Tobacco mosaic virus leader (TMV), (Gallie et al., 1989; and Maize Chlorotic Mottle Virus leader (MCMV) (Lommel et al., 1991. See also, Della-Cioppa et al., 1987.

Regulatory elements such as Adh intron 1 (Callis et al., 1987), sucrose synthase intron (Vasil et al., 1989) or TMV omega element (Gallie, et al., 1989), may further be included where desired.

Examples of enhancers include elements from the CaMV 35S promoter, octopine synthase genes (Ellis et al., 1987), the rice actin I gene, the maize alcohol dehydrogenase gene (Callis et al., 1987), the maize shrunken I gene (Vasil et al., 1989), TMV Omega element (Gallie et al., 1989) and promoters from non-plant eukaryotes (e.g. yeast; Ma et al., 1988).

Vectors for use in accordance with the present invention may be constructed to include the ocs enhancer element. This element was first identified as a 16 bp palindromic enhancer from the octopine synthase (ocs) gene of ultilane (Ellis et al., 1987), and is present in at least 10 other promoters (Bouchez et al., 1989). The use of an enhancer element, such as the ocs element and particularly multiple copies of the element, will act to increase the level of transcription from adjacent promoters when applied in the context of monocot transformation.

Ultimately, the most desirable DNA segments for introduction into for example a monocot genome may be homologous genes or gene families which encode a desired trait (e.g., increased yield per acre) and which are introduced under the control of novel promoters or enhancers, etc., or perhaps even homologous or tissue specific (e.g., root-, collar/sheath-, whorl-, stalk-, earshank-, kernel- or leaf-specific) promoters or control elements. Indeed, it is envisioned that a particular use of the present invention will be the targeting of a gene in a constitutive manner or a root-specific manner. For example, insect resistant genes may be expressed specifically in the whorl and collar/sheath tissues which are targets for the first and second broods, respectively, of ECB. Likewise, genes encoding proteins with particular activity against rootworm may be targeted directly to root tissues.

Vectors for use in tissue-specific targeting of genes in transgenic plants will typically include tissue-specific promoters and may also include other tissue-specific control elements such as enhancer sequences. Promoters which direct specific or enhanced expression in certain plant tissues will be known to those of skill in the art in light of the present disclosure. These include, for example, the rbcS promoter, specific for green tissue; the ocs, nos and mas promoters which have higher activity in roots or wounded leaf tissue; a truncated (−90 to +8) 35S promoter which directs enhanced expression in roots, an alpha-tubulin gene that directs expression in roots and promoters derived from zein storage protein genes which direct expression in endosperm. It is particularly contemplated that one may advantageously use the 16 bp ocs enhancer element from the octopine synthase (ocs) gene (Ellis et al., 1987; Bouchez et al., 1989), especially when present in multiple copies, to achieve enhanced expression in roots.

Tissue specific expression may be functionally accomplished by introducing a constitutively expressed gene (all tissues) in combination with an antisense gene that is expressed only in those tissues where the gene product is not desired. For example, a gene coding for the crystal toxin protein from B. thuringiensis (Bt) may be introduced such that it is expressed in all tissues using the 35S promoter from Cauliflower Mosaic Virus. Expression of an antisense transcript of the Bt gene in a maize kernel, using for example a zein promoter, would prevent accumulation of the Bt protein in seed. Hence the protein encoded by the introduced gene would be present in all tissues except the kernel.

Expression of some genes in transgenic plants will be desired only under specified conditions. For example, it is proposed that expression of certain genes that confer resistance to environmental stress factors such as drought will be desired only under actual stress conditions. It is contemplated that expression of such genes throughout a plants development may have detrimental effects. It is known that a large number of genes exist that respond to the environment. For example, expression of some genes such as rbcS, encoding the small subunit of ribulose bisphosphate carboxylase, is regulated by light as mediated through phytochrome. Other genes are induced by secondary stimuli. For example, synthesis of abscisic acid (ABA) is induced by certain environmental factors, including but not limited to water stress. A number of genes have been shown to be induced by ABA (Skriver and Mundy, 1990). It is also anticipated that expression of genes conferring resistance to insect predation would be desired only under conditions of actual insect infestation. Therefore, for some desired traits inducible expression of genes in transgenic plants will be desired.

Expression of a gene in a transgenic plant will be desired only in a certain time period during the development of the plant. Developmental timing is frequently correlated with tissue specific gene expression. For example, expression of zein storage proteins is initiated in the endosperm about 15 days after pollination.

Additionally, vectors may be constructed and employed in the intracellular targeting of a specific gene product within the cells of a transgenic plant or in directing a protein to the extracellular environment. This will generally be achieved by joining a DNA sequence encoding a transit or signal peptide sequence to the coding sequence of a particular gene. The resultant transit, or signal, peptide will transport the protein to a particular intracellular, or extracellular destination, respectively, and will then be post-translationally removed. Transit or signal peptides act by facilitating the transport of proteins through intracellular membranes, e.g., vacuole, vesicle, plastid and mitochondrial membranes, whereas signal peptides direct proteins through the extracellular membrane.

A particular example of such a use concerns the direction of a herbicide resistance gene, such as the EPSPS gene, to a particular organelle such as the chloroplast rather than to the cytoplasm. This is exemplified by the use of the rbcs transit peptide which confers plastid-specific targeting of proteins. In addition, it is proposed that it may be desirable to target certain genes responsible for male sterility to the mitochondria, or to target certain genes for resistance to phytopathogenic organisms to the extracellular spaces, or to target proteins to the vacuole.

By facilitating the transport of the protein into compartments inside and outside the cell, these sequences may increase the accumulation of gene product protecting them from proteolytic degradation. These sequences also allow for additional mRNA sequences from highly expressed genes to be attached to the coding sequence of the genes. Since mRNA being translated by ribosomes is more stable than naked mRNA, the presence of translatable mRNA in front of the gene may increase the overall stability of the mRNA transcript from the gene and thereby increase synthesis of the gene product. Since transit and signal sequences are usually post-translationally removed from the initial translation product, the use of these sequences allows for the addition of extra translated sequences that may not appear on the final polypeptide. Targeting of certain proteins may be desirable in order to enhance the stability of the protein (U.S. Pat. No. 5,545,818).

It may be useful to target DNA itself within a cell. For example, it may be useful to target introduced DNA to the nucleus as this may increase the frequency of transformation. Within the nucleus itself it would be useful to target a gene in order to achieve site specific integration. For example, it would be useful to have an gene introduced through transformation replace an existing gene in the cell.

Other elements include those that can be regulated by endogenous or exogenous agents, e.g., by zinc finger proteins, including naturally occurring zinc finger proteins or chimeric zinc finger proteins (see, e.g., U.S. Pat. No. 5,789,538, WO 99/48909; WO 99/45132; WO 98/53060; WO 98/53057; WO 98/53058; WO 00/23464; WO 95/19431; and WO 98/54311) or myb-like transcription factors. For example, a chimeric zinc finger protein may include amino acid sequences which bind to a specific DNA sequence (the zinc finger) and amino acid sequences that activate (e.g., GAL 4 sequences) or repress the transcription of the sequences linked to the specific DNA sequence.

3. Preferred Nucleic Acid Molecules of the Invention

The invention relates to an isolated plant, e.g., Arabidopsis, Chenopodium and rice, nucleic acid molecule comprising a gene having an open reading frame, the expression of which is altered in response to pathogen infection, as well as the endogenous plant promoters for those genes. However, the expression of these genes may also be altered in response to non-pathogens, e.g., in response to environmental stiumuli. The nucleic acid molecules can be used in pathogen control strategies, e.g., by overexpressing nucleic acid molecules which can confer tolerance to a cell, or by altering the expression of host genes which are required for pathogen infection, e.g., by “knocking out” the expression of at least one genomic copy of the gene. Plants having genetic disruptions in host genes may be less susceptible to infection, e.g., due to a decrease or absence of a host protein needed for infection, or, alternatively, hypersusceptible to infection. Plants that are hypersusceptible to infection may be useful to prepare transgenic plants as the expression of the gene(s) which was disrupted may be related to gene silencing.

Preferred sources from which the nucleic acid molecules of the invention can be obtained or isolated include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassaya (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea ultilane), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, duckweed (Lemna), barley, vegetables, ornamentals, and conifers.

Duckweed (Lemna, see WO 00/07210) includes members of the family Lemnaceae. There are known four genera and 34 species of duckweed as follows: genus Lemna (L. aequinoctialis, L. disperma, L. ecuadoriensis, L. gibba, L. japonica, L. minor, L. miniscula, L. obscura, L. perpusilla, L. tenera, L. trisulca, L. turionifera, L. valdiviana); genus Spirodela (S. intermedia, S. polyrrhiza, S. punctata); genus Woffia (Wa. Angusta, Wa. Arrhiza, Wa. Australina, Wa. Borealis, Wa. Brasiliensis, Wa. Columbiana, Wa. Elongata, Wa. Globosa, Wa. Microscopica, Wa. Neglecta) and genus Wofiella (Wl. ultila, Wl. ultilane n, Wl. gladiata, Wl. ultila, Wl. lingulata, Wl. repunda, Wl. rotunda, and Wl. neotropica). Any other genera or species of Lemnaceae, if they exist, are also aspects of the present invention. Lemna gibba, Lemna minor, and Lemna miniscula are preferred, with Lemna minor and Lemna miniscula being most preferred. Lemna species can be classified using the taxonomic scheme described by Landolt, Biosystematic Investigation on the Family of Duckweeds: The family of Lemnaceae—A Monograph Study. Geobatanischen Institut ETH, Stiftung Rubel, Zurich (1986)).

Vegetables from which to obtain or isolate the nucleic acid molecules of the invention include, but are not limited to, tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals from which to obtain or isolate the nucleic acid molecules of the invention include, but are not limited to, azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata), Douglas-fir (Pseudotsuga menziesii); Western hemlock (Tsuga ultilane); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). Leguminous plants from which the nucleic acid molecules of the invention can be isolated or obtained include, but are not limited to, beans and peas. Beans include guar, locust bean, fenugreek, soybean, garden beans, cowpea, mungbean, lima bean, fava bean, lentils, chickpea, and the like. Legumes include, but are not limited to, Arachis, e.g., peanuts, Vicia, e.g., crown vetch, hairy vetch, adzuki bean, mung bean, and chickpea, Lupinus, e.g., lupine, trifolium, Phaseolus, e.g., common bean and lima bean, Pisum, e.g., field bean, Melilotus, e.g., clover, Medicago, e.g., alfalfa, Lotus, e.g., trefoil, lens, e.g., lentil, and false indigo.

Papaya, garlic, pea, peach, pepper, petunia, strawberry, sorghum, sweet potato, turnip, safflower, corn, pea, endive, gourd, grape, snap bean, chicory, cotton, tobacco, aubergine, beet, buckwheat, broad bean, nectarine, avocado, mango, banana, groundnut, potato, peanut, lettuce, pineapple, spinach, squash, sugarbeet, sugarcane, sweet corn, chrysanthemum.

Other preferred sources of the nucleic acid molecules of the invention include Acacia, aneth, artichoke, arugula, blackberry, canola, cilantro, clementines, escarole, eucalyptus, fennel, grapefruit, honey dew, jicama, kiwifruit, lemon, lime, mushroom, nut, okra, orange, parsley, persimmon, plantain, pomegranate, poplar, radiata pine, radicchio, Southern pine, sweetgum, tangerine, triticale, vine, yams, apple, pear, quince, cherry, apricot, melon, hemp, buckwheat, grape, raspberry, chenopodium, blueberry, nectarine, peach, plum, strawberry, watermelon, eggplant, pepper, cauliflower, Brassica, e.g., broccoli, cabbage, ultilan sprouts, onion, carrot, leek, beet, broad bean, celery, radish, pumpkin, endive, gourd, garlic, snapbean, spinach, squash, turnip, ultilane, and zucchini.

Yet other sources of nucleic acid molecules are ornamental plants including, but not limited to, impatiens, Begonia, Pelargonium, Viola, Cyclamen, Verbena, Vinca, Tagetes, Primula, Saint Paulia, Agertum, Amaranthus, Antihirrhinum, Aquilegia, Cineraria, Clover, Cosmo, Cowpea, Dahlia, Datura, Delphinium, Gerbera, Gladiolus, Gloxinia, Hippeastrum, Mesembryanthemum, Salpiglossos, and Zinnia, and plants such as those shown in Table 1.

TABLE 1

| | LATIN | COMMON | MAP REFERENCES | |
|---|---|---|---|---|
| FAMILY | NAME | NAME | RESOURCES | LINKS |
| Cucurbitaceae | Cucumis | Cucumber | | http://www.cucurbit.org/ |
| | sativus | | | |
| | Cucumis | Melon | | http://genome.cornell.edu/cg/c |
| | melo | | | |
| | Citrullus | Watermelon | | |
| | lanatus | | | |
| | Cucurbita | Squash - | | |
| | pepo | summer | | |
| | Cucurbita | Squash - | | |
| | maxima | winter | | |
| | Cucurbita | Pumpkin/ | | |
| | moschata | butternut | | |
| Total | | | | http://www.nal.usda.gov/pgdic/ |
| | | | | Map_proj/ |
| Solanaceae | Lycopersicon | Tomato | 15x BAC on variety | genome.cornell.edu/solgenes |
| | esculentum | | Heinz 1706 order from | http://ars-genome.cornell.edu/cgi- |
| | | | Clemson Genome center | bin/WebAce/webace?db=solgenes |
| | | | (www.genome.clemson.edu) | http://genome.cornell.edu/tgc/ |
| | | | 11.6x BAC of L. cheesmanii | http://tgrc.ucdavis.edu/ |
| | | | (originates | |
| | | | from J. Giovannoni) | |
| | | | available from Clemson | |
| | | | genome center | |
| | | | (www.genome.clemson.edu) | |
| | | | EST collection from | |
| | | | TIGR | |
| | | | (www.tigr.org/tdb/lgi/index.html) | |
| | | | EST collection from | |
| | | | Clemsom Genome | |
| | | | Center | |
| | | | (www.genome.clemson.edu) | |
| | | | TAG 99: 254-271, 1999 | |
| | | | (esculentum x pennelli) | |
| | | | TAG 89: 1007-1013, | |
| | | | 1994 (peruvianum) | |
| | | | Plant Cell Reports | |
| | | | 12: 293-297, 1993 | |
| | | | (RAPDs) | |
| | | | Genetics 132: 1141-1160, | |
| | | | 1992 (potato x tomato) | |
| | | | Genetics 120: 1095-1105, | |
| | | | 1988 (RFLP potato and | |
| | | | tomato) | |
| | | | Genetics 115: 387-393, | |
| | | | 1986 (esculentum x pennelli | |
| | | | isozyme and | |
| | | | cDNAs) | |
| | Capsicum | Pepper | | http://neptune.netimages.com/˜chile/ |
| | annuum | | | science.html |
| | Capsicum | Chile pepper | | |
| | frutescens | | | |
| | Solanum | Eggplant | | |
| | melongena | | | |
| | (Nicotiana | (Tobacco) | | |
| | tabacum) | | | |
| | (Solanum | (Potato) | | |
| | tuberosum) | | | |
| | (Petunia x hybrida | (hort.Petunia) | 4x BAC of Petunia hybrida | |
| | hort. | | 7984 available from | |
| | Ex E. Vilm.) | | Clemson genome center | |
| | | | (www.genome.clemson.edu) | |
| Total | | | | http://www.nal.usda.gov/pgdic/ |
| | | | | Map_proj/ |
| Brassicaceae | Brassica | Broccoli | | http://res.agr.ca/ecorc/cwmt/crucifer/ |
| | oleracea L. | | | traits/index.htm |
| | var. italica | | | http://geneous.cit.cornell.edu/ |
| | Brassica | Cabbage | | cabbage/aboutcab.html |
| | oleracea L. | | | |
| | var. capitata | | | |
| | Brassica | Chinese | | |
| | rapa | Cabbage | | |
| | Brassica | Cauliflower | | |
| | oleracea L. | | | |
| | var. botrytis | | | |
| | Raphanus | Daikon | | |
| | sativus var. | | | |
| | niger | | | |
| | (Brassica | (Oilseed | | http://ars-genome.cornell.edu/cgi-bin/ |
| | napus) | rape) | | WebAce/webace?db=brassicadb |
| | | Arabidopsis | 12x and 6x BACs on | http://ars-genome.cornell.edu/cgi-bin/ |
| | | | Columbia strain available | WebAce/webace?db=agr |
| | | | from Clemson genome | |
| | | | center | |
| | | | (www.genome.clemson.edu) | |
| Total | | | | http://www.nal.usda.gov/pgdic/ |
| | | | | Map_proj/ |
| Umbelliferae | Daucus | Carrot | | |
| | carota | | | |
| Compositae | Lactuca | Lettuce | | |
| | sativa | | | |
| | Helianthus | (Sunflower) | | |
| | annuus | | | |
| Total | | | | |
| Chenopodiaceae | Spinacia | Spinach | | |
| | oleracea | | | |
| | (Beta | (Sugar Beet) | | |
| | vulgaris) | | | |
| Total | | | | |
| Leguminosae | Phaseolus | Bean | 4.3x BAC available from | http://ars-genome.cornell.edu/cgi-bin/ |
| | vulgaris | | Clemson genome center | WebAce/webace?db=beangenes |
| | | | (www.genome.clemson.edu) | |
| | Pisum | Pea | | |
| | sativum | | | |
| | (Glycine | (Soybean) | 7.5x and 7.9x BACs | http://ars-genome.cornell.edu/cgi-bin/ |
| | max) | | available from Clemson | WebAce/webace?db=soybase |
| | | | genome center | |
| | | | (www.genome.clemson.edu) | |
| Total | | | http://www.nal.usda.gov/pgdic/Map_proj/ | |
| Gramineae | Zea mays | Sweet Corn | Novartis BACs for Mo17 | |
| | | | and B73 have been donated | |
| | | | to Clemson Genome Center | |
| | | | (www.genome.clemson.edu) | |
| | (Zea mays) | (Field Corn) | | http://www.agron.missouri.edu/mnl/ |
| Total | | | http://www.nal.usda.gov/pgdic/Map_proj/ | |
| Liliaceae | Allium cepa | Onion | | |
| | | Leek | | |
| | | (Garlic) | | |
| | | (Asparagus) | | |
| Total | | | http://www.nal.usda.gov/pgdic/Map_proj/ | |

Preferred forage and turf grass nucleic acid sources for the nucleic acid molecules of the invention include, but are not limited to, alfalfa, orchard grass, tall fescue, perennial ryegrass, creeping bent grass, and redtop. Yet other preferred sources include, but are not limited to, crop plants and in particular cereals (for example, corn, alfalfa, sunflower, rice, Brassica, canola, soybean, barley, soybean, sugarbeet, cotton, safflower, peanut, sorghum, oat, rye, rape, wheat, millet, tobacco, and the like), and even more preferably corn, rice and soybean.

According to one embodiment, the present invention is directed to a nucleic acid molecule comprising a nucleotide sequence isolated or obtained from any plant which encodes a polypeptide having at least 70% amino acid sequence identity to a polypeptide encoded by a gene comprising any one of SEQ ID NOs:1-953, 1954-1966, 2000-2129 or 2662-4737, or a gene comprising SEQ ID NOs:1001-1094, 2137-2661 or 4738-6813. Based on the Arabidopsis, Chenopdoium and rice nucleic acid sequences of the present invention, orthologs may be identified or isolated from the genome of any desired organism, preferably from another plant, according to well known techniques based on their sequence similarity to the Arabidopsis, Chenopodium and rice nucleic acid sequences, e.g., hybridization, PCR or computer generated sequence comparisons. For example, all or a portion of a particular Arabidopsis, Chenopodium and rice nucleic acid sequence is used as a probe that selectively hybridizes to other gene sequences present in a population of cloned genomic DNA fragments or cDNA fragments (i.e., genomic or cDNA libraries) from a chosen source organism. Further, suitable genomic and cDNA libraries may be prepared from any cell or tissue of an organism. Such techniques include hybridization screening of plated DNA libraries (either plaques or colonies; see, e.g., Sambrook et al., 1989) and amplification by PCR using oligonucleotide primers preferably corresponding to sequence domains conserved among related polypeptide or subsequences of the nucleotide sequences provided herein (see, e.g., Innis et al., 1990). These methods are particularly well suited to the isolation of gene sequences from organisms closely related to the organism from which the probe sequence is derived. The application of these methods using the Arabidopsis sequences as probes is well suited for the isolation of gene sequences from any source organism, preferably other plant species. In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art.

In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as ³²P, or any other detectable marker. Thus, for example, probes for hybridization can be made by labeling synthetic oligonucleotides based on the sequence of the invention. Methods for preparation of probes for hybridization and for construction of cDNA and genomic libraries are generally known in the art and are disclosed in Sambrook et al. (1989). In general, sequences that hybridize to the sequences disclosed herein will have at least 40% to 50%, about 60% to 70% and even about 80% 85%, 90%, 95% to 98% or more identity with the disclosed sequences. That is, the sequence similarity of sequences may range, sharing at least about 40% to 50%, about 60% to 70%, and even about 80%, 85%, 90%, 95% to 98% sequence similarity.

The nucleic acid molecules of the invention can also be identified by, for example, a search of known databases for genes encoding polypeptides having a specified amino acid sequence identity or DNA having a specified nucleotide sequence identity. Methods of alignment of sequences for comparison are well known in the art and are described hereinabove.

4. Methods for Mutagenizing DNA

It is specifically contemplated by the inventors that one could mutagenize DNA having a promoter or open reading frame to, for example, potentially improve the utility of the DNA for expression of transgenes in plants. The mutagenesis can be carried out at random and the mutagenized sequences screened for activity in a trial-by-error procedure. Alternatively, particular sequences which provide the promoter with desirable expression characteristics, or a promoter with expression enhancement activity, could be identified and these or similar sequences introduced into the sequences via mutation. It is further contemplated that one could mutagenize these sequences in order to enhance their expression of transgenes in a particular species.

The means for mutagenizing a DNA segment of the current invention are well-known to those of skill in the art. As indicated, modifications may be made by random or site-specific mutagenesis procedures. The DNA may be modified by altering its structure through the addition or deletion of one or more nucleotides from the sequence which encodes the corresponding unmodified sequences.

Mutagenesis may be performed in accordance with any of the techniques known in the art, such as, and not limited to, synthesizing an oligonucleotide having one or more mutations within the sequence of a particular regulatory region. In particular, site-specific mutagenesis is a technique useful in the preparation of promoter mutants, through specific mutagenesis of the underlying DNA. The technique further provides a ready ability to prepare and test sequence variants, for example, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to about 75 nucleotides or more in length is preferred, with about 10 to about 25 or more residues on both sides of the junction of the sequence being altered.

In general, the technique of site-specific mutagenesis is well known in the art, as exemplified by various publications. As will be appreciated, the technique typically employs a phage vector which exists in both a single stranded and double stranded form. Typical vectors useful in site-directed mutagenesis include vectors such as the M13 phage. These phage are readily commercially available and their use is generally well known to those skilled in the art.

Double stranded plasmids also are routinely employed in site directed mutagenesis which eliminates the step of transferring the gene of interest from a plasmid to a phage.

In general, site-directed mutagenesis in accordance herewith is performed by first obtaining a single-stranded vector or melting apart of two strands of a double stranded vector which includes within its sequence a DNA sequence which encodes the promoter. An oligonucleotide primer bearing the desired mutated sequence is prepared, generally synthetically. This primer is then annealed with the single-stranded vector, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation.

This heteroduplex vector is then used to transform or transfect appropriate cells, such as E. coli cells, and cells are selected which include recombinant vectors bearing the mutated sequence arrangement. Vector DNA can then be isolated from these cells and used for plant transformation. A genetic selection scheme was devised by Kunkel et al. (1987) to enrich for clones incorporating mutagenic oligonucleotides. Alternatively, the use of PCR with commercially available thermostable enzymes such as Taq polymerase may be used to incorporate a mutagenic oligonucleotide primer into an amplified DNA fragment that can then be cloned into an appropriate cloning or expression vector. The PCR-mediated mutagenesis procedures of Tomic et al. (1990) and Upender et al. (1995) provide two examples of such protocols. A PCR employing a thermostable ligase in addition to a thermostable polymerase also may be used to incorporate a phosphorylated mutagenic oligonucleotide into an amplified DNA fragment that may then be cloned into an appropriate cloning or expression vector. The mutagenesis procedure described by Michael (1994) provides an example of one such protocol.

The preparation of sequence variants of DNA segments using site-directed mutagenesis is provided as a means of producing potentially useful species and is not meant to be limiting as there are other ways in which sequence variants of DNA sequences may be obtained. For example, recombinant vectors encoding the desired promoter sequence may be treated with mutagenic agents, such as hydroxylamine, to obtain sequence variants.

In addition, an unmodified or modified nucleotide sequence of the present invention can be varied by shuffling the sequence of the invention. To test for a function of variant DNA sequences according to the invention, the sequence of interest is operably linked to a selectable or screenable marker gene and expression of the marker gene is tested in transient expression assays with protoplasts or in stably transformed plants. It is known to the skilled artisan that DNA sequences capable of driving expression of an associated nucleotide sequence are build in a modular way. Accordingly, expression levels from shorter DNA fragments may be different than the one from the longest fragment and may be different from each other. For example, deletion of a down-regulating upstream element will lead to an increase in the expression levels of the associated nucleotide sequence while deletion of an up-regulating element will decrease the expression levels of the associated nucleotide sequence. It is also known to the skilled artisan that deletion of development-specific or a tissue-specific element will lead to a temporally or spatially altered expression profile of the associated nucleotide sequence.

As used herein, the term “oligonucleotide directed mutagenesis procedure” refers to template-dependent processes and vector-mediated propagation which result in an increase in the concentration of a specific nucleic acid molecule relative to its initial concentration, or in an increase in the concentration of a detectable signal, such as amplification. As used herein, the term “oligonucleotide directed mutagenesis procedure” also is intended to refer to a process that involves the template-dependent extension of a primer molecule. The term template-dependent process refers to nucleic acid synthesis of an RNA or a DNA molecule wherein the sequence of the newly synthesized strand of nucleic acid is dictated by the well-known rules of complementary base pairing (see, for example, Watson and Rarnstad, 1987). Typically, vector mediated methodologies involve the introduction of the nucleic acid fragment into a DNA or RNA vector, the clonal amplification of the vector, and the recovery of the amplified nucleic acid fragment. Examples of such methodologies are provided by U.S. Pat. No. 4,237,224. A number of template dependent processes are available to amplify the target sequences of interest present in a sample, such methods being well known in the art and specifically disclosed herein below.

Where a clone comprising a promoter has been isolated in accordance with the instant invention, one may wish to delimit the essential promoter regions within the clone. One efficient, targeted means for preparing mutagenizing promoters relies upon the identification of putative regulatory elements within the promoter sequence. This can be initiated by comparison with promoter sequences known to be expressed in similar tissue-specific or developmentally unique manner. Sequences which are shared among promoters with similar expression patterns are likely candidates for the binding of transcription factors and are thus likely elements which confer expression patterns. Confirmation of these putative regulatory elements can be achieved by deletion analysis of each putative regulatory region followed by functional analysis of each deletion construct by assay of a reporter gene which is functionally attached to each construct. As such, once a starting promoter sequence is provided, any of a number of different deletion mutants of the starting promoter could be readily prepared.

As indicated above, deletion mutants, deletion mutants of the promoter of the invention also could be randomly prepared and then assayed. With this strategy, a series of constructs are prepared, each containing a different portion of the clone (a subclone), and these constructs are then screened for activity. A suitable means for screening for activity is to attach a deleted promoter or intron construct which contains a deleted segment to a selectable or screenable marker, and to isolate only those cells expressing the marker gene. In this way, a number of different, deleted promoter constructs are identified which still retain the desired, or even enhanced, activity. The smallest segment which is required for activity is thereby identified through comparison of the selected constructs. This segment may then be used for the construction of vectors for the expression of exogenous genes.

B. Marker Genes

In order to improve the ability to identify transformants, one may desire to employ a selectable or screenable marker gene as, or in addition to, the expressible gene of interest. “Marker genes” are genes that impart a distinct phenotype to cells expressing the marker gene and thus allow such transformed cells to be distinguished from cells that do not have the marker. Such genes may encode either a selectable or screenable marker, depending on whether the marker confers a trait which one can ‘select’ for by chemical means, i.e., through the use of a selective agent (e.g., a herbicide, antibiotic, or the like), or whether it is simply a trait that one can identify through observation or testing, i.e., by ‘screening’ (e.g., the R-locus trait, the green fluorescent protein (GFP)). Of course, many examples of suitable marker genes are known to the art and can be employed in the practice of the invention.

Included within the terms selectable or screenable marker genes are also genes which encode a “secretable marker” whose secretion can be detected as a means of identifying or selecting for transformed cells. Examples include markers which encode a secretable antigen that can be identified by antibody interaction, or even secretable enzymes which can be detected by their catalytic activity. Secretable proteins fall into a number of classes, including small, diffusible proteins detectable, e.g., by ELISA; small active enzymes detectable in extracellular solution (e.g., alpha-amylase, beta-lactamase, phosphinothricin acetyltransferase); and proteins that are inserted or trapped in the cell wall (e.g., proteins that include a leader sequence such as that found in the expression unit of extensin or tobacco PR-S).

With regard to selectable secretable markers, the use of a gene that encodes a protein that becomes sequestered in the cell wall, and which protein includes a unique epitope is considered to be particularly advantageous. Such a secreted antigen marker would ideally employ an epitope sequence that would provide low background in plant tissue, a promoter-leader sequence that would impart efficient expression and targeting across the plasma membrane, and would produce protein that is bound in the cell wall and yet accessible to antibodies. A normally secreted wall protein modified to include a unique epitope would satisfy all such requirements.

One example of a protein suitable for modification in this manner is extensin, or hydroxyproline rich glycoprotein (HPRG). For example, the maize HPRG (Steifel et al., 1990) molecule is well characterized in terms of molecular biology, expression and protein structure. However, any one of a variety of ultilane and/or glycine-rich wall proteins (Keller et al., 1989) could be modified by the addition of an antigenic site to create a screenable marker.

One exemplary embodiment of a secretable screenable marker concerns the use of a maize sequence encoding the wall protein HPRG, modified to include a 15 residue epitope from the pro-region of murine interleukin, however, virtually any detectable epitope may be employed in such embodiments, as selected from the extremely wide variety of antigen-antibody combinations known to those of skill in the art. The unique extracellular epitope can then be straightforwardly detected using antibody labeling in conjunction with chromogenic or fluorescent adjuncts.

Elements of the present disclosure may be exemplified in detail through the use of the bar and/or GUS genes, and also through the use of various other markers. Of course, in light of this disclosure, numerous other possible selectable and/or screenable marker genes will be apparent to those of skill in the art in addition to the one set forth hereinbelow. Therefore, it will be understood that the following discussion is exemplary rather than exhaustive. In light of the techniques disclosed herein and the general recombinant techniques which are known in the art, the present invention renders possible the introduction of any gene, including marker genes, into a recipient cell to generate a transformed plant.

1. Selectable Markers

Possible selectable markers for use in connection with the present invention include, but are not limited to, a neo gene which codes for kanamycin resistance and can be selected for using kanamycin, G418, paromomycin, and the like; a bar gene which codes for bialaphos or phosphinothricin resistance; a gene which encodes an altered EPSP synthase protein (Hinchee et al., 1988) thus conferring glyphosate resistance; a nitrilase gene such as bxn from Klebsiella ozaenae which confers resistance to bromoxynil (Stalker et al., 1988); a mutant acetolactate synthase gene (ALS) which confers resistance to imidazolinone, sulfonylurea or other ALS-inhibiting chemicals (European Patent Application 154,204, 1985); a methotrexate-resistant DHFR gene (Thillet et al., 1988); a dalapon dehalogenase gene that confers resistance to the herbicide dalapon; a mutated anthranilate synthase gene that confers resistance to 5-methyl tryptophan. Preferred selectable marker genes encode phosphinothricin acetyltransferase; glyphosate resistant EPSPS, aminoglycoside phosphotransferase; hygromycin phosphotransferase, or neomycin phosphotransferase. Where a mutant EPSP synthase gene is employed, additional benefit may be realized through the incorporation of a suitable chloroplast transit peptide, CTP (European Patent Application 0,218,571, 1987).

An illustrative embodiment of a selectable marker gene capable of being used in systems to select transformants is the genes that encode the enzyme phosphinothricin acetyltransferase, such as the bar gene from Streptomyces hygroscopicus or the pat gene from Streptomyces viridochromogenes. The enzyme phosphinothricin acetyl transferase (PAT) inactivates the active ingredient in the herbicide bialaphos, phosphinothricin (PPT). PPT inhibits glutamine synthetase, (Murakami et al., 1986; Twell et al., 1989) causing rapid accumulation of ammonia and cell death. The success in using this selective system in conjunction with monocots was particularly surprising because of the major difficulties which have been reported in transformation of cereals.

Where one desires to employ a bialaphos resistance gene in the practice of the invention, a particularly useful gene for this purpose is the bar or pat genes obtainable from species of Streptomyces (e.g., ATCC No. 21,705). The cloning of the bar gene has been described (Murakami et al., 1986; Thompson et al., 1987) as has the use of the bar gene in the context of plants other than monocots (De Block et al., 1987; De Block et al., 1989).

Selection markers resulting in positive selection, such as a phosphomannose isomerase gene, as described in patent application WO 93/05163, may also be used. Alternative genes to be used for positive selection are described in WO 94/20627 and encode xyloisomerases and phosphomanno-isomerases such as mannose-6-phosphate isomerase and mannose-1-phosphate isomerase; phosphomanno mutase; mannose epimerases such as those which convert carbohydrates to mannose or mannose to carbohydrates such as glucose or galactose; phosphatases such as mannose or xylose phosphatase, mannose-6-phosphatase and mannose-1-phosphatase, and permeases which are involved in the transport of mannose, or a derivative, or a precursor thereof into the cell. Transformed cells are identified without damaging or killing the non-transformed cells in the population and without co-introduction of antibiotic or herbicide resistance genes. As described in WO 93/05163, in addition to the fact that the need for antibiotic or herbicide resistance genes is eliminated, it has been shown that the positive selection method is often far more efficient than traditional negative selection.

2. Screenable Markers

Screenable markers that may be employed include, but are not limited to, a beta-glucuronidase (GUS) or uidA gene which encodes an enzyme for which various chromogenic substrates are known; an R-locus gene, which encodes a product that regulates the production of anthocyanin pigments (red color) in plant tissues (Dellaporta et al., 1988); a beta-lactamase gene (Sutcliffe, 1978), which encodes an enzyme for which various chromogenic substrates are known (e.g., PADAC, a chromogenic cephalosporin); a xylE gene (Zukowsky et al., 1983) which encodes a catechol dioxygenase that can convert chromogenic catechols; an α-amylase gene (Ikuta et al., 1990); a tyrosinase gene (Katz et al., 1983) which encodes an enzyme capable of oxidizing tyrosine to DOPA and dopaquinone which in turn condenses to form the easily detectable compound melanin; a P-galactosidase gene, which encodes an enzyme for which there are chromogenic substrates; a luciferase (lux) gene (Ow et al., 1986), which allows for bioluminescence detection; or even an aequorin gene (Prasher et al., 1985), which may be employed in calcium-sensitive bioluminescence detection, or a green fluorescent protein gene (Niedz et al., 1995).

Genes from the maize R gene complex are contemplated to be particularly useful as screenable markers. The R gene complex in maize encodes a protein that acts to regulate the production of anthocyanin pigments in most seed and plant tissue. A gene from the R gene complex was applied to maize transformation, because the expression of this gene in transformed cells does not harm the cells. Thus, an R gene introduced into such cells will cause the expression of a red pigment and, if stably incorporated, can be visually scored as a red sector. If a maize line is carries dominant ultila for genes encoding the enzymatic intermediates in the anthocyanin biosynthetic pathway (C2, A1, A2, Bz1 and Bz2) (Roth et al., 1990), but carries a recessive allele at the R locus, transformation of any cell from that line with R will result in red pigment formation. Exemplary lines include Wisconsin 22 which contains the rg-Stadler allele and TR112, a K55 derivative which is r-g, b, P1. Alternatively any genotype of maize can be utilized if the C1 and R alleles are introduced together.

It is further proposed that R gene regulatory regions may be employed in chimeric constructs in order to provide mechanisms for controlling the expression of chimeric genes. More diversity of phenotypic expression is known at the R locus than at any other locus (Coe et al., 1988). It is contemplated that regulatory regions obtained from regions 5′ to the structural R gene would be valuable in directing the expression of genes, e.g., insect resistance, drought resistance, herbicide tolerance or other protein coding regions. For the purposes of the present invention, it is believed that any of the various R gene family members may be successfully employed (e.g., P, S, Lc, etc.). However, the most preferred will generally be Sn (particularly Sn:bol3). Sn is a dominant member of the R gene complex and is functionally similar to the R and B loci in that Sn controls the tissue specific deposition of anthocyanin pigments in certain seedling and plant cells, therefore, its phenotype is similar to R.

A further screenable marker contemplated for use in the present invention is firefly luciferase, encoded by the lux gene. The presence of the lux gene in transformed cells may be detected using, for example, X-ray film, scintillation counting, fluorescent spectrophotometry, low-light video cameras, photon counting cameras or multiwell luminometry. It is also envisioned that this system may be developed for populational screening for bioluminescence, such as on tissue culture plates, or even for whole plant screening. Where use of a screenable marker gene such as lux or GFP is desired, benefit may be realized by creating a gene fusion between the screenable marker gene and a selectable marker gene, for example, a GFP-NPTII gene fusion. This could allow, for example, selection of transformed cells followed by screening of transgenic plants or seeds.

C. Exogenous Genes for Modification of Plant Phenotypes

Genes of interest are reflective of the commercial markets and interests of those involved in the development of the crop. Crops and markets of interest changes, and as developing nations open up world markets, new crops and technologies will also emerge. In addition, as the understanding of agronomic traits and characteristics such as yield and heterosis increase, the choice of genes for transformation will change accordingly. General categories of genes of interest include, for example, those genes involved in information, such as zinc fingers, those involved in communication, such as kinases, and those involved in housekeeping, such as heat shock proteins. More specific categories of transgenes, for example, include genes encoding important traits for agronomics, insect resistance, disease resistance, herbicide resistance, sterility, grain characteristics, and commercial products. Genes of interest include, generally, those involved in starch, oil, carbohydrate, or nutrient metabolism, as well as those affecting kernel size, sucrose loading, zinc finger proteins, see, e.g., U.S. Pat. No. 5,789,538, WO 99/48909; WO 99/45132; WO 98/53060; WO 98/53057; WO 98/53058; WO 00/23464; WO 95/19431; and WO 98/54311, and the like.

One skilled in the art recognizes that the expression level and regulation of a transgene in a plant can vary significantly from line to line. Thus, one has to test several lines to find one with the desired expression level and regulation. Once a line is identified with the desired regulation specificity of a chimeric Cre transgene, it can be crossed with lines carrying different inactive replicons or inactive transgene for activation.

Other sequences which may be linked to the gene of interest which encodes a polypeptide are those which can target to a specific organelle, e.g., to the mitochondria, nucleus, or plastid, within the plant cell. Targeting can be achieved by providing the polypeptide with an appropriate targeting peptide sequence, such as a secretory signal peptide (for secretion or cell wall or membrane targeting, a plastid transit peptide, a chloroplast transit peptide, e.g., the chlorophyll a/b binding protein, a mitochondrial target peptide, a vacuole targeting peptide, or a nuclear targeting peptide, and the like. For example, the small subunit of ribulose bisphosphate carboxylase transit peptide, the EPSPS transit peptide or the dihydrodipicolinic acid synthase transit peptide may be used. For examples of plastid organelle targeting sequences (see WO 00/12732). Plastids are a class of plant organelles derived from proplastids and include chloroplasts, leucoplasts, aravloplasts, and chromoplasts. The plastids are major sites of biosynthesis in plants. In addition to photosynthesis in the chloroplast, plastids are also sites of lipid biosynthesis, nitrate reduction to ammonium, and starch storage. And while plastids contain their own circular genome, most of the proteins localized to the plastids are encoded by the nuclear genome and are imported into the organelle from the cytoplasm.

Transgenes used with the present invention will often be genes that direct the expression of a particular protein or polypeptide product, but they may also be non-expressible DNA segments, e.g., transposons such as Ds that do no direct their own transposition. As used herein, an “expressible gene” is any gene that is capable of being transcribed into RNA (e.g., mRNA, antisense RNA, etc.) or translated into a protein, expressed as a trait of interest, or the like, etc., and is not limited to selectable, screenable or non-selectable marker genes. The invention also contemplates that, where both an expressible gene that is not necessarily a marker gene is employed in combination with a marker gene, one may employ the separate genes on either the same or different DNA segments for transformation. In the latter case, the different vectors are delivered concurrently to recipient cells to maximize cotransformation.

The choice of the particular DNA segments to be delivered to the recipient cells will often depend on the purpose of the transformation. One of the major purposes of transformation of crop plants is to add some commercially desirable, agronomically important traits to the plant. Such traits include, but are not limited to, herbicide resistance or tolerance; insect resistance or tolerance; disease resistance or tolerance (viral, bacterial, fungal, nematode); stress tolerance and/or resistance, as exemplified by resistance or tolerance to drought, heat, chilling, freezing, excessive moisture, salt stress; oxidative stress; increased yields; food content and makeup; physical appearance; male sterility; drydown; standability; prolificacy; starch properties; oil quantity and quality; and the like. One may desire to incorporate one or more genes conferring any such desirable trait or traits, such as, for example, a gene or genes encoding pathogen resistance.

In certain embodiments, the present invention contemplates the transformation of a recipient cell with more than one advantageous transgene. Two or more transgenes can be supplied in a single transformation event using either distinct transgene-encoding vectors, or using a single vector incorporating two or more gene coding sequences. For example, plasmids bearing the bar and aroA expression units in either convergent, divergent, or colinear orientation, are considered to be particularly useful. Further preferred combinations are those of an insect resistance gene, such as a Bt gene, along with a protease inhibitor gene such as pinII, or the use of bar in combination with either of the above genes. Of course, any two or more transgenes of any description, such as those conferring herbicide, insect, disease (viral, bacterial, fungal, nematode) or drought resistance, male sterility, drydown, standability, prolificacy, starch properties, oil quantity and quality, or those increasing yield or nutritional quality may be employed as desired.

1. Herbicide Resistance

The genes encoding phosphinothricin acetyltransferase (bar and pat), glyphosate tolerant EPSP synthase genes, the glyphosate degradative enzyme gene gox encoding glyphosate oxidoreductase, deh (encoding a dehalogenase enzyme that inactivates dalapon), herbicide resistant (e.g., sulfonylurea and imidazolinone) acetolactate synthase, and bxn genes (encoding a nitrilase enzyme that degrades bromoxynil) are good examples of herbicide resistant genes for use in transformation. The bar and pat genes code for an enzyme, phosphinothricin acetyltransferase (PAT), which inactivates the herbicide phosphinothricin and prevents this compound from inhibiting glutamine synthetase enzymes. The enzyme 5-enolpyruvylshikimate 3-phosphate synthase (EPSP Synthase), is normally inhibited by the herbicide N-(phosphonomethyl)glycine (glyphosate). However, genes are known that encode glyphosate-resistant EPSP Synthase enzymes.

These genes are particularly contemplated for use in monocot transformation. The deh gene encodes the enzyme dalapon dehalogenase and confers resistance to the herbicide dalapon. The bxn gene codes for a specific nitrilase enzyme that converts bromoxynil to a non-herbicidal degradation product.

2. Insect Resistance

An important aspect of the present invention concerns the introduction of insect resistance-conferring genes into plants. Potential insect resistance genes which can be introduced include Bacillus thuringiensis crystal toxin genes or Bt genes (Watrud et al., 1985). Bt genes may provide resistance to lepidopteran or coleopteran pests such as European Corn Borer (ECB) and corn rootworm (CRW). Preferred Bt toxin genes for use in such embodiments include the CryIA(b) and CryIA(c) genes. Endotoxin genes from other species of B. thuringiensis which affect insect growth or development may also be employed in this regard.

The poor expression of Bt toxin genes in plants is a well-documented phenomenon, and the use of different promoters, fusion proteins, and leader sequences has not led to significant increases in Bt protein expression (Vaeck et al., 1989; Barton et al., 1987). It is therefore contemplated that the most advantageous Bt genes for use in the transformation protocols disclosed herein will be those in which the coding sequence has been modified to effect increased expression in plants, and more particularly, those in which maize preferred codons have been used. Examples of such modified Bt toxin genes include the variant Bt CryIA(b) gene termed Iab6 (Perlak et al., 1991) and the synthetic CryIA(c) genes termed 1800a and 1800b.

Protease inhibitors may also provide insect resistance (Johnson et al., 1989), and will thus have utility in plant transformation. The use of a protease inhibitor II gene, pinII, from tomato or potato is envisioned to be particularly useful. Even more advantageous is the use of a pinII gene in combination with a Bt toxin gene, the combined effect of which has been discovered by the present inventors to produce synergistic insecticidal activity. Other genes which encode inhibitors of the insects' digestive system, or those that encode enzymes or co-factors that facilitate the production of inhibitors, may also be useful. This group may be exemplified by oryzacystatin and amylase inhibitors, such as those from wheat and barley.

Also, genes encoding lectins may confer additional or alternative insecticide properties. Lectins (originally termed phytohemagglutinins) are multivalent carbohydrate-binding proteins which have the ability to agglutinate red blood cells from a range of species. Lectins have been identified recently as insecticidal agents with activity against weevils, ECB and rootworm (Murdock et al., 1990; Czapla and Lang, 1990). Lectin genes contemplated to be useful include, for example, barley and wheat germ agglutinin (WGA) and rice lectins (Gatehouse et al., 1984), with WGA being preferred.

Genes controlling the production of large or small polypeptides active against insects when introduced into the insect pests, such as, e.g., lytic peptides, peptide hormones and toxins and venoms, form another aspect of the invention. For example, it is contemplated that the expression of juvenile hormone esterase, directed towards specific insect pests, may also result in insecticidal activity, or perhaps cause cessation of metamorphosis (Hammock et al., 1990).

Transgenic plants expressing genes which encode enzymes that affect the integrity of the insect cuticle form yet another aspect of the invention. Such genes include those encoding, e.g., chitinase, proteases, lipases and also genes for the production of nikkomycin, a compound that inhibits chitin synthesis, the introduction of any of which is contemplated to produce insect resistant maize plants. Genes that code for activities that affect insect molting, such those affecting the production of ecdysteroid UDP-glucosyl transferase, also fall within the scope of the useful transgenes of the present invention.

Genes that code for enzymes that facilitate the production of compounds that reduce the nutritional quality of the host plant to insect pests are also encompassed by the present invention. It may be possible, for instance, to confer insecticidal activity on a plant by altering its sterol composition. Sterols are obtained by insects from their diet and are used for hormone synthesis and membrane stability. Therefore alterations in plant sterol composition by expression of novel genes, e.g., those that directly promote the production of undesirable sterols or those that convert desirable sterols into undesirable forms, could have a negative effect on insect growth and/or development and hence endow the plant with insecticidal activity. Lipoxygenases are naturally occurring plant enzymes that have been shown to exhibit anti-nutritional effects on insects and to reduce the nutritional quality of their diet. Therefore, further embodiments of the invention concern transgenic plants with enhanced lipoxygenase activity which may be resistant to insect feeding.

The present invention also provides methods and compositions by which to achieve qualitative or quantitative changes in plant secondary metabolites. One example concerns transforming plants to produce DIMBOA which, it is contemplated, will confer resistance to European corn borer, rootworm and several other maize insect pests. Candidate genes that are particularly considered for use in this regard include those genes at the bx locus known to be involved in the synthetic DIMBOA pathway (Dunn et al., 1981). The introduction of genes that can regulate the production of maysin, and genes involved in the production of dhurrin in sorghum, is also contemplated to be of use in facilitating resistance to earworm and rootworm, respectively.

Tripsacum dactyloides is a species of grass that is resistant to certain insects, including corn root worm. It is anticipated that genes encoding proteins that are toxic to insects or are involved in the biosynthesis of compounds toxic to insects will be isolated from Tripsacum and that these novel genes will be useful in conferring resistance to insects. It is known that the basis of insect resistance in Tripsacum is genetic, because said resistance has been transferred to Zea mays via sexual crosses (Branson and Guss, 1972).

Further genes encoding proteins characterized as having potential insecticidal activity may also be used as transgenes in accordance herewith. Such genes include, for example, the cowpea trypsin inhibitor (CpTI; Hilder et al., 1987) which may be used as a rootworm deterrent; genes encoding avermectin (Campbell, 1989; Ikeda et al., 1987) which may prove particularly useful as a corn rootworm deterrent; ribosome inactivating protein genes; and even genes that regulate plant structures. Transgenic maize including anti-insect antibody genes and genes that code for enzymes that can covert a non-toxic insecticide (pro-insecticide) applied to the outside of the plant into an insecticide inside the plant are also contemplated.

3. Environment or Stress Resistance

Improvement of a plant's ability to tolerate various environmental stresses such as, but not limited to, drought, excess moisture, chilling, freezing, high temperature, salt, and oxidative stress, can also be effected through expression of heterologous, or overexpression of homologous genes. Benefits may be realized in terms of increased resistance to freezing temperatures through the introduction of an “antifreeze” protein such as that of the Winter Flounder (Cutler et al., 1989) or synthetic gene derivatives thereof. Improved chilling tolerance may also be conferred through increased expression of glycerol-3-phosphate acetyltransferase in chloroplasts (Murata et al., 1992; Wolter et al., 1992). Resistance to oxidative stress (often exacerbated by conditions such as chilling temperatures in combination with high light intensities) can be conferred by expression of superoxide dismutase (Gupta et al., 1993), and may be improved by glutathione reductase (Bowler et al., 1992). Such strategies may allow for tolerance to freezing in newly emerged fields as well as extending later maturity higher yielding varieties to earlier relative maturity zones.

Expression of novel genes that favorably effect plant water content, total water potential, osmotic potential, and turgor can enhance the ability of the plant to tolerate drought. As used herein, the terms “drought resistance” and “drought tolerance” are used to refer to a plants increased resistance or tolerance to stress induced by a reduction in water availability, as compared to normal circumstances, and the ability of the plant to function and survive in lower-water environments, and perform in a relatively superior manner. In this aspect of the invention it is proposed, for example, that the expression of a gene encoding the biosynthesis of osmotically-active solutes can impart protection against drought. Within this class of genes are DNAs encoding mannitol dehydrogenase (Lee and Saier, 1982) and trehalose-6-phosphate synthase (Kaasen et al., 1992). Through the subsequent action of native phosphatases in the cell or by the introduction and coexpression of a specific phosphatase, these introduced genes will result in the accumulation of either mannitol or trehalose, respectively, both of which have been well documented as protective compounds able to mitigate the effects of stress. Mannitol accumulation in transgenic tobacco has been verified and preliminary results indicate that plants expressing high levels of this metabolite are able to tolerate an applied osmotic stress (Tarczynski et al., cited supra (1992), 1993).

Similarly, the efficacy of other metabolites in protecting either enzyme function (e.g. alanopine or propionic acid) or membrane integrity (e.g., alanopine) has been documented (Loomis et al., 1989), and therefore expression of gene encoding the biosynthesis of these compounds can confer drought resistance in a manner similar to or complimentary to mannitol. Other examples of naturally occurring metabolites that are osmotically active and/or provide some direct protective effect during drought and/or desiccation include sugars and sugar derivatives such as fructose, erythritol (Coxson et al., 1992), sorbitol, dulcitol (Karsten et al., 1992), glucosylglycerol (Reed et al., 1984; Erdmann et al., 1992), sucrose, stachyose (Koster and Leopold, 1988; Blackman et al., 1992), ononitol and pinitol (Vernon and Bohnert, 1992), and raffinose (Bemal-Lugo and Leopold, 1992). Other osmotically active solutes which are not sugars include, but are not limited to, proline and glycine-betaine (Wyn-Jones and Storey, 1981). Continued canopy growth and increased reproductive fitness during times of stress can be augmented by introduction and expression of genes such as those controlling the osmotically active compounds discussed above and other such compounds, as represented in one exemplary embodiment by the enzyme myoinositol O-methyltransferase.

It is contemplated that the expression of specific proteins may also increase drought tolerance. Three classes of Late Embryogenic Proteins have been assigned based on structural similarities (see Dure et al., 1989). All three classes of these proteins have been demonstrated in maturing (i.e., desiccating) seeds. Within these 3 types of proteins, the Type-II (dehydrin-type) have generally been implicated in drought and/or desiccation tolerance in vegetative plant parts (i.e. Mundy and Chua, 1988; Piatkowski et al., 1990; Yamaguchi-Shinozaki et al., 1992). Recently, expression of a Type-III LEA (HVA-1) in tobacco was found to influence plant height, maturity and drought tolerance (Fitzpatrick, 1993). Expression of structural genes from all three groups may therefore confer drought tolerance. Other types of proteins induced during water stress include thiol proteases, aldolases and transmembrane transporters (Guerrero et al., 1990), which may confer various protective and/or repair-type functions during drought stress. The expression of a gene that effects lipid biosynthesis and hence membrane composition can also be useful in conferring drought resistance on the plant.

Many genes that improve drought resistance have complementary modes of action. Thus, combinations of these genes might have additive and/or synergistic effects in improving drought resistance in plants. Many of these genes also improve freezing tolerance (or resistance); the physical stresses incurred during freezing and drought are similar in nature and may be mitigated in similar fashion. Benefit may be conferred via constitutive expression of these genes, but the preferred means of expressing these novel genes may be through the use of a turgor-induced promoter (such as the promoters for the turgor-induced genes described in Guerrero et al. 1990 and Shagan et al., 1993). Spatial and temporal expression patterns of these genes may enable maize to better withstand stress.

Expression of genes that are involved with specific morphological traits that allow for increased water extractions from drying soil would be of benefit. For example, introduction and expression of genes that alter root characteristics may enhance water uptake. Expression of genes that enhance reproductive fitness during times of stress would be of significant value. For example, expression of DNAs that improve the synchrony of pollen shed and receptiveness of the female flower parts, i.e., silks, would be of benefit. In addition, expression of genes that minimize kernel abortion during times of stress would increase the amount of grain to be harvested and hence be of value. Regulation of cytokinin levels in monocots, such as maize, by introduction and expression of an isopentenyl transferase gene with appropriate regulatory sequences can improve monocot stress resistance and yield (Gan et al., Science, 270:1986 (1995)).

Given the overall role of water in determining yield, it is contemplated that enabling plants to utilize water more efficiently, through the introduction and expression of novel genes, will improve overall performance even when soil water availability is not limiting. By introducing genes that improve the ability of plants to maximize water usage across a full range of stresses relating to water availability, yield stability or consistency of yield performance may be realized.

4. Disease Resistance

It is proposed that increased resistance to diseases may be realized through introduction of genes into plants period. It is possible to produce resistance to diseases caused by viruses, bacteria, fungi, root pathogens, insects and nematodes. It is also contemplated that control of mycotoxin producing organisms may be realized through expression of introduced genes.

Resistance to viruses may be produced through expression of novel genes. For example, it has been demonstrated that expression of a viral coat protein in a transgenic plant can impart resistance to infection of the plant by that virus and perhaps other closely related viruses (Cuozzo et al., 1988, Hemenway et al., 1988, Abel et al., 1986). It is contemplated that expression of antisense genes targeted at essential viral functions may impart resistance to said virus. For example, an antisense gene targeted at the gene responsible for replication of viral nucleic acid may inhibit said replication and lead to resistance to the virus. It is believed that interference with other viral functions through the use of antisense genes may also increase resistance to viruses. Further it is proposed that it may be possible to achieve resistance to viruses through other approaches, including, but not limited to the use of satellite viruses.

It is proposed that increased resistance to diseases caused by bacteria and fungi may be realized through introduction of novel genes. It is contemplated that genes encoding so-called “peptide antibiotics,” pathogenesis related (PR) proteins, toxin resistance, and proteins affecting host-pathogen interactions such as morphological characteristics will be useful. Peptide antibiotics are polypeptide sequences which are inhibitory to growth of bacteria and other microorganisms. For example, the classes of peptides referred to as cecropins and magainins inhibit growth of many species of bacteria and fungi. It is proposed that expression of PR proteins in plants may be useful in conferring resistance to bacterial disease. These genes are induced following pathogen attack on a host plant and have been divided into at least five classes of proteins (Bol et al., 1990). Included amongst the PR proteins are beta-1,3-glucanases, chitinases, and osmotin and other proteins that are believed to function in plant resistance to disease organisms. Other genes have been identified that have antifungal properties, e.g., UDA (stinging nettle lectin) and hevein (Broakgert et al., 1989; Barkai-Golan et al., 1978). It is known that certain plant diseases are caused by the production of phytotoxins. Resistance to these diseases could be achieved through expression of a novel gene that encodes an enzyme capable of degrading or otherwise inactivating the phytotoxin. Expression novel genes that alter the interactions between the host plant and pathogen may be useful in reducing the ability the disease organism to invade the tissues of the host plant, e.g., an increase in the waxiness of the leaf cuticle or other morphological characteristics.

Plant parasitic nematodes are a cause of disease in many plants. It is proposed that it would be possible to make the plant resistant to these organisms through the expression of novel genes. It is anticipated that control of nematode infestations would be accomplished by altering the ability of the nematode to recognize or attach to a host plant and/or enabling the plant to produce nematicidal compounds, including but not limited to proteins.

5. Mycotoxin Reduction/Elimination

Production of mycotoxins, including aflatoxin and fumonisin, by fungi associated with plants is a significant factor in rendering the grain not useful. These fungal organisms do not cause disease symptoms and/or interfere with the growth of the plant, but they produce chemicals (mycotoxins) that are toxic to animals. Inhibition of the growth of these fungi would reduce the synthesis of these toxic substances and, therefore, reduce grain losses due to mycotoxin contamination. Novel genes may be introduced into plants that would inhibit synthesis of the mycotoxin without interfering with fungal growth. Expression of a novel gene which encodes an enzyme capable of rendering the mycotoxin nontoxic would be useful in order to achieve reduced mycotoxin contamination of grain. The result of any of the above mechanisms would be a reduced presence of mycotoxins on grain.

6. Grain Composition or Quality

Genes may be introduced into plants, particularly commercially important cereals such as maize, wheat or rice, to improve the grain for which the cereal is primarily grown. A wide range of novel transgenic plants produced in this manner may be envisioned depending on the particular end use of the grain.

For example, the largest use of maize grain is for feed or food. Introduction of genes that alter the composition of the grain may greatly enhance the feed or food value. The primary components of maize grain are starch, protein, and oil. Each of these primary components of maize grain may be improved by altering its level or composition. Several examples may be mentioned for illustrative purposes but in no way provide an exhaustive list of possibilities.

The protein of many cereal grains is suboptimal for feed and food purposes especially when fed to pigs, poultry, and humans. The protein is deficient in several amino acids that are essential in the diet of these species, requiring the addition of supplements to the grain. Limiting essential amino acids may include lysine, methionine, tryptophan, threonine, valine, arginine, and histidine. Some amino acids become limiting only after the grain is supplemented with other inputs for feed formulations. For example, when the grain is supplemented with soybean meal to meet lysine requirements, methionine becomes limiting. The levels of these essential amino acids in seeds and grain may be elevated by mechanisms which include, but are not limited to, the introduction of genes to increase the biosynthesis of the amino acids, decrease the degradation of the amino acids, increase the storage of the amino acids in proteins, or increase transport of the amino acids to the seeds or grain.

One mechanism for increasing the biosynthesis of the amino acids is to introduce genes that deregulate the amino acid biosynthetic pathways such that the plant can no longer adequately control the levels that are produced. This may be done by deregulating or bypassing steps in the amino acid biosynthetic pathway which are normally regulated by levels of the amino acid end product of the pathway. Examples include the introduction of genes that encode deregulated versions of the enzymes aspartokinase or dihydrodipicolinic acid (DHDP)-synthase for increasing lysine and threonine production, and anthranilate synthase for increasing tryptophan production. Reduction of the catabolism of the amino acids may be accomplished by introduction of DNA sequences that reduce or eliminate the expression of genes encoding enzymes that catalyse steps in the catabolic pathways such as the enzyme lysine-ketoglutarate reductase.

The protein composition of the grain may be altered to improve the balance of amino acids in a variety of ways including elevating expression of native proteins, decreasing expression of those with poor composition, changing the composition of native proteins, or introducing genes encoding entirely new proteins possessing superior composition. DNA may be introduced that decreases the expression of members of the zein family of storage proteins. This DNA may encode ribozymes or antisense sequences directed to impairing expression of zein proteins or expression of regulators of zein expression such as the opaque-2 gene product. The protein composition of the grain may be modified through the phenomenon of cosuppression, i.e., inhibition of expression of an endogenous gene through the expression of an identical structural gene or gene fragment introduced through transformation (Goring et al., 1991). Additionally, the introduced DNA may encode enzymes which degrade seines. The decreases in zein expression that are achieved may be accompanied by increases in proteins with more desirable amino acid composition or increases in other major seed constituents such as starch. Alternatively, a chimeric gene may be introduced that comprises a coding sequence for a native protein of adequate amino acid composition such as for one of the globulin proteins or 10 kD zein of maize and a promoter or other regulatory sequence designed to elevate expression of said protein. The coding sequence of said gene may include additional or replacement codons for essential amino acids. Further, a coding sequence obtained from another species, or, a partially or completely synthetic sequence encoding a completely unique peptide sequence designed to enhance the amino acid composition of the seed may be employed.

The introduction of genes that alter the oil content of the grain may be of value. Increases in oil content may result in increases in metabolizable energy content and density of the seeds for uses in feed and food. The introduced genes may encode enzymes that remove or reduce rate-limitations or regulated steps in fatty acid or lipid biosynthesis. Such genes may include, but are not limited to, those that encode acetyl-CoA carboxylase, ACP-acyltransferase, beta-ketoacyl-ACP synthase, plus other well known fatty acid biosynthetic activities. Other possibilities are genes that encode proteins that do not possess enzymatic activity such as acyl carrier protein. Additional examples include 2-acetyltransferase, oleosin pyruvate dehydrogenase complex, acetyl CoA synthetase, ATP citrate lyase, ADP-glucose pyrophosphorylase and genes of the carnitine-CoA-acetyl-CoA shuttles. It is anticipated that expression of genes related to oil biosynthesis will be targeted to the plastid, using a plastid transit peptide sequence and preferably expressed in the seed embryo. Genes may be introduced that alter the balance of fatty acids present in the oil providing a more healthful or nutritive feedstuff. The introduced DNA may also encode sequences that block expression of enzymes involved in fatty acid biosynthesis, altering the proportions of fatty acids present in the grain such as described below.

Genes may be introduced that enhance the nutritive value of the starch component of the grain, for example by increasing the degree of branching, resulting in improved utilization of the starch in cows by delaying its metabolism.

Besides affecting the major constituents of the grain, genes may be introduced that affect a variety of other nutritive, processing, or other quality aspects of the grain as used for feed or food. For example, pigmentation of the grain may be increased or decreased. Enhancement and stability of yellow pigmentation is desirable in some animal feeds and may be achieved by introduction of genes that result in enhanced production of xanthophylls and carotenes by eliminating rate-limiting steps in their production. Such genes may encode altered forms of the enzymes phytoene synthase, phytoene desaturase, or lycopene synthase. Alternatively, unpigmented white corn is desirable for production of many food products and may be produced by the introduction of DNA which blocks or eliminates steps in pigment production pathways.

Feed or food comprising some cereal grains possesses insufficient quantities of vitamins and must be supplemented to provide adequate nutritive value. Introduction of genes that enhance vitamin biosynthesis in seeds may be envisioned including, for example, vitamins A, E, B₁₂, choline, and the like. For example, maize grain also does not possess sufficient mineral content for optimal nutritive value. Genes that affect the accumulation or availability of compounds containing phosphorus, sulfur, calcium, manganese, zinc, and iron among others would be valuable. An example may be the introduction of a gene that reduced phytic acid production or encoded the enzyme phytase which enhances phytic acid breakdown. These genes would increase levels of available phosphate in the diet, reducing the need for supplementation with mineral phosphate.

Numerous other examples of improvement of cereals for feed and food purposes might be described. The improvements may not even necessarily involve the grain, but may, for example, improve the value of the grain for silage. Introduction of DNA to accomplish this might include sequences that alter lignin production such as those that result in the “brown midrib” phenotype associated with superior feed value for cattle.

In addition to direct improvements in feed or food value, genes may also be introduced which improve the processing of grain and improve the value of the products resulting from the processing. The primary method of processing certain grains such as maize is via wetmilling. Maize may be improved though the expression of novel genes that increase the efficiency and reduce the cost of processing such as by decreasing steeping time.

Improving the value of wetmilling products may include altering the quantity or quality of starch, oil, corn gluten meal, or the components of corn gluten feed. Elevation of starch may be achieved through the identification and elimination of rate limiting steps in starch biosynthesis or by decreasing levels of the other components of the grain resulting in proportional increases in starch. An example of the former may be the introduction of genes encoding ADP-glucose pyrophosphorylase enzymes with altered regulatory activity or which are expressed at higher level. Examples of the latter may include selective inhibitors of, for example, protein or oil biosynthesis expressed during later stages of kernel development.

The properties of starch may be beneficially altered by changing the ratio of amylose to amylopectin, the size of the starch molecules, or their branching pattern. Through these changes a broad range of properties may be modified which include, but are not limited to, changes in gelatinization temperature, heat of gelatinization, clarity of films and pastes, Theological properties, and the like. To accomplish these changes in properties, genes that encode granule-bound or soluble starch synthase activity or branching enzyme activity may be introduced alone or combination. DNA such as antisense constructs may also be used to decrease levels of endogenous activity of these enzymes. The introduced genes or constructs may possess regulatory sequences that time their expression to specific intervals in starch biosynthesis and starch granule development. Furthermore, it may be advisable to introduce and express genes that result in the in vivo derivatization, or other modification, of the glucose moieties of the starch molecule. The covalent attachment of any molecule may be envisioned, limited only by the existence of enzymes that catalyze the derivatizations and the accessibility of appropriate substrates in the starch granule. Examples of important derivations may include the addition of functional groups such as amines, carboxyls, or phosphate groups which provide sites for subsequent in vitro derivatizations or affect starch properties through the introduction of ionic charges. Examples of other modifications may include direct changes of the glucose units such as loss of hydroxyl groups or their oxidation to aldehyde or carboxyl groups.

Oil is another product of wetmilling of corn and other grains, the value of which may be improved by introduction and expression of genes. The quantity of oil that can be extracted by wetmilling may be elevated by approaches as described for feed and food above. Oil properties may also be altered to improve its performance in the production and use of cooking oil, shortenings, lubricants or other oil-derived products or improvement of its health attributes when used in the food-related applications. Novel fatty acids may also be synthesized which upon extraction can serve as starting materials for chemical syntheses. The changes in oil properties may be achieved by altering the type, level, or lipid arrangement of the fatty acids present in the oil. This in turn may be accomplished by the addition of genes that encode enzymes that catalyze the synthesis of novel fatty acids and the lipids possessing them or by increasing levels of native fatty acids while possibly reducing levels of precursors. Alternatively DNA sequences may be introduced which slow or block steps in fatty acid biosynthesis resulting in the increase in precursor fatty acid intermediates. Genes that might be added include desaturases, epoxidases, hydratases, dehydratases, and other enzymes that catalyze reactions involving fatty acid intermediates. Representative examples of catalytic steps that might be blocked include the desaturations from stearic to oleic acid and oleic to linolenic acid resulting in the respective accumulations of stearic and oleic acids.

Improvements in the other major cereal wetmilling products, gluten meal and gluten feed, may also be achieved by the introduction of genes to obtain novel plants. Representative possibilities include but are not limited to those described above for improvement of food and feed value.

In addition it may further be considered that the plant be used for the production or manufacturing of useful biological compounds that were either not produced at all, or not produced at the same level, in the plant previously. The novel plants producing these compounds are made possible by the introduction and expression of genes by transformation methods. The possibilities include, but are not limited to, any biological compound which is presently produced by any organism such as proteins, nucleic acids, primary and intermediary metabolites, carbohydrate polymers, etc. The compounds may be produced by the plant, extracted upon harvest and/or processing, and used for any presently recognized useful purpose such as pharmaceuticals, fragrances, industrial enzymes to name a few.

Further possibilities to exemplify the range of grain traits or properties potentially encoded by introduced genes in transgenic plants include grain with less breakage susceptibility for export purposes or larger grit size when processed by dry milling through introduction of genes that enhance gamma-zein synthesis, popcorn with improved popping quality and expansion volume through genes that increase pericarp thickness, corn with whiter grain for food uses though introduction of genes that effectively block expression of enzymes involved in pigment production pathways, and improved quality of alcoholic beverages or sweet corn through introduction of genes which affect flavor such as the shrunken gene (encoding sucrose synthase) for sweet corn.

7. Plant Agronomic Characteristics

Two of the factors determining where plants can be grown are the average daily temperature during the growing season and the length of time between frosts. Within the areas where it is possible to grow a particular plant, there are varying limitations on the maximal time it is allowed to grow to maturity and be harvested. The plant to be grown in a particular area is selected for its ability to mature and dry down to harvestable moisture content within the required period of time with maximum possible yield. Therefore, plant of varying maturities are developed for different growing locations. Apart from the need to dry down sufficiently to permit harvest is the desirability of having maximal drying take place in the field to minimize the amount of energy required for additional drying post-harvest. Also the more readily the grain can dry down, the more time there is available for growth and kernel fill. Genes that influence maturity and/or dry down can be identified and introduced into plant lines using transformation techniques to create new varieties adapted to different growing locations or the same growing location but having improved yield to moisture ratio at harvest. Expression of genes that are involved in regulation of plant development may be especially useful, e.g., the liguleless and rough sheath genes that have been identified in plants.

Genes may be introduced into plants that would improve standability and other plant growth characteristics. For example, expression of novel genes which confer stronger stalks, improved root systems, or prevent or reduce ear droppage would be of great value to the corn farmer. Introduction and expression of genes that increase the total amount of photoassimilate available by, for example, increasing light distribution and/or interception would be advantageous. In addition the expression of genes that increase the efficiency of photosynthesis and/or the leaf canopy would further increase gains in productivity. Such approaches would allow for increased plant populations in the field.

Delay of late season vegetative senescence would increase the flow of assimilate into the grain and thus increase yield. Overexpression of genes within plants that are associated with “stay green” or the expression of any gene that delays senescence would achieve be advantageous. For example, a non-yellowing mutant has been identified in Festuca pratensis (Davies et al., 1990). Expression of this gene as well as others may prevent premature breakdown of chlorophyll and thus maintain canopy function.

8. Nutrient Utilization

The ability to utilize available nutrients and minerals may be a limiting factor in growth of many plants. It is proposed that it would be possible to alter nutrient uptake, tolerate pH extremes, mobilization through the plant, storage pools, and availability for metabolic activities by the introduction of novel genes. These modifications would allow a plant to more efficiently utilize available nutrients. It is contemplated that an increase in the activity of, for example, an enzyme that is normally present in the plant and involved in nutrient utilization would increase the availability of a nutrient. An example of such an enzyme would be phytase. It is also contemplated that expression of a novel gene may make a nutrient source available that was previously not accessible, e.g., an enzyme that releases a component of nutrient value from a more complex molecule, perhaps a macromolecule.

9. Male Sterility

Male sterility is useful in the production of hybrid seed. It is proposed that male sterility may be produced through expression of novel genes. For example, it has been shown that expression of genes that encode proteins that interfere with development of the male inflorescence and/or gametophyte result in male sterility. Chimeric ribonuclease genes that express in the anthers of transgenic tobacco and oilseed rape have been demonstrated to lead to male sterility (Mariani et al, 1990).

For example, a number of mutations were discovered in maize that confer cytoplasmic male sterility. One mutation in particular, referred to as T cytoplasm, also correlates with sensitivity to Southern corn leaf blight. A DNA sequence, designated TURF-13 (Levings, 1990), was identified that correlates with T cytoplasm. It would be possible through the introduction of TURF-13 via transformation to separate male sterility from disease sensitivity. As it is necessary to be able to restore male fertility for breeding purposes and for grain production, it is proposed that genes encoding restoration of male fertility may also be introduced.

10. Negative Selectable Markers

Introduction of genes encoding traits that can be selected against may be useful for eliminating undesirable linked genes. When two or more genes are introduced together by cotransformation, the genes will be linked together on the host chromosome. For example, a gene encoding a Bt gene that confers insect resistance on the plant may be introduced into a plant together with a bar gene that is useful as a selectable marker and confers resistance to the herbicide Ignite® on the plant. However, it may not be desirable to have an insect resistant plant that is also resistant to the herbicide Ignite®. It is proposed that one could also introduce an antisense bar gene that is expressed in those tissues where one does not want expression of the bar gene, e.g., in whole plant parts. Hence, although the bar gene is expressed and is useful as a selectable marker, it is not useful to confer herbicide resistance on the whole plant. The bar antisense gene is a negative selectable marker.

Negative selection is necessary in order to screen a population of transformants for rare homologous recombinants generated through gene targeting. For example, a homologous recombinant may be identified through the inactivation of a gene that was previously expressed in that cell. The antisense gene to neomycin phosphotransferase II (nptII) has been investigated as a negative selectable marker in tobacco (Nicotiana tabacum) and Arabidopsis thaliana (Xiang and Guerra, 1993). In this example both sense and antisense nptII genes are introduced into a plant through transformation and the resultant plants are sensitive to the antibiotic kanamycin. An introduced gene that integrates into the host cell chromosome at the site of the antisense nptII gene, and inactivates the antisense gene, will make the plant resistant to kanamycin and other aminoglycoside antibiotics. Therefore, rare site specific recombinants may be identified by screening for antibiotic resistance. Similarly, any gene, native to the plant or introduced through transformation, that when inactivated confers resistance to a compound, may be useful as a negative selectable marker.

It is contemplated that negative selectable markers may also be useful in other ways. One application is to construct transgenic lines in which one could select for transposition to unlinked sites. In the process of tagging it is most common for the transposable element to move to a genetically linked site on the same chromosome. A selectable marker for recovery of rare plants in which transposition has occurred to an unlinked locus would be useful. For example, the enzyme cytosine deaminase may be useful for this purpose (Stouggard, 1993). In the presence of this enzyme the compound 5-fluorocytosine is converted to 5-fluoruracil which is toxic to plant and animal cells. If a transposable element is linked to the gene for the enzyme cytosine deaminase, one may select for transposition to unlinked sites by selecting for transposition events in which the resultant plant is now resistant to 5-fluorocytosine. The parental plants and plants containing transpositions to linked sites will remain sensitive to 5-fluorocytosine. Resistance to 5-fluorocytosine is due to loss of the cytosine deaminase gene through genetic segregation of the transposable element and the cytosine deaminase gene. Other genes that encode proteins that render the plant sensitive to a certain compound will also be useful in this context. For example, T-DNA gene 2 from Agrobacterium tumefaciens encodes a protein that catalyzes the conversion of alpha-naphthalene acetamide (NAM) to alpha-napthalene acetic acid (NAA) renders plant cells sensitive to high concentrations of NAM (Depicker et al., 1988).

It is also contemplated that negative selectable markers may be useful in the construction of transposon tagging lines. For example, by marking an autonomous transposable element such as Ac, Master Mu, or En/Spn with a negative selectable marker, one could select for transformants in which the autonomous element is not stably integrated into the genome. This would be desirable, for example, when transient expression of the autonomous element is desired to activate in trans the transposition of a defective transposable element, such as Ds, but stable integration of the autonomous element is not desired. The presence of the autonomous element may not be desired in order to stabilize the defective element, i.e., prevent it from further transposing. However, it is proposed that if stable integration of an autonomous transposable element is desired in a plant the presence of a negative selectable marker may make it possible to eliminate the autonomous element during the breeding process.

11. Non-Protein-Expressing Sequences

a. RNA-Expressing

DNA may be introduced into plants for the purpose of expressing RNA transcripts that function to affect plant phenotype yet are not translated into protein. Two examples are antisense RNA and RNA with ribozyme activity. Both may serve possible functions in reducing or eliminating expression of native or introduced plant genes.

Genes may be constructed or isolated, which when transcribed, produce antisense RNA that is complementary to all or part(s) of a targeted messenger RNA(s). The antisense RNA reduces production of the polypeptide product of the messenger RNA. The polypeptide product may be any protein encoded by the plant genome. The aforementioned genes will be referred to as antisense genes. An antisense gene may thus be introduced into a plant by transformation methods to produce a novel transgenic plant with reduced expression of a selected protein of interest. For example, the protein may be an enzyme that catalyzes a reaction in the plant. Reduction of the enzyme activity may reduce or eliminate products of the reaction which include any enzymatically synthesized compound in the plant such as fatty acids, amino acids, carbohydrates, nucleic acids and the like. Alternatively, the protein may be a storage protein, such as a zein, or a structural protein, the decreased expression of which may lead to changes in seed amino acid composition or plant morphological changes respectively. The possibilities cited above are provided only by way of example and do not represent the full range of applications.

Genes may also be constructed or isolated, which when transcribed produce RNA enzymes, or ribozymes, which can act as endoribonucleases and catalyze the cleavage of RNA molecules with selected sequences. The cleavage of selected messenger RNA's can result in the reduced production of their encoded polypeptide products. These genes may be used to prepare novel transgenic plants which possess them. The transgenic plants may possess reduced levels of polypeptides including but not limited to the polypeptides cited above that may be affected by antisense RNA.

It is also possible that genes may be introduced to produce novel transgenic plants which have reduced expression of a native gene product by a mechanism of cosuppression. It has been demonstrated in tobacco, tomato, and petunia (Goring et al, 1991; Smith et al., 1990; Napoli et al., 1990; van der Krol et al., 1990) that expression of the sense transcript of a native gene will reduce or eliminate expression of the native gene in a manner similar to that observed for antisense genes. The introduced gene may encode all or part of the targeted native protein but its translation may not be required for reduction of levels of that native protein.

b. Non-RNA-Expressing

For example, DNA elements including those of transposable elements such as Ds, Ac, or Mu, may be inserted into a gene and cause mutations. These DNA elements may be inserted in order to inactivate (or activate) a gene and thereby “tag” a particular trait. In this instance the transposable element does not cause instability of the tagged mutation, because the utility of the element does not depend on its ability to move in the genome. Once a desired trait is tagged, the introduced DNA sequence may be used to clone the corresponding gene, e.g., using the introduced DNA sequence as a PCR primer together with PCR gene cloning techniques (Shapiro, 1983; Dellaporta et al., 1988). Once identified, the entire gene(s) for the particular trait, including control or regulatory regions where desired may be isolated, cloned and manipulated as desired. The utility of DNA elements introduced into an organism for purposed of gene tagging is independent of the DNA sequence and does not depend on any biological activity of the DNA sequence, i.e., transcription into RNA or translation into protein. The sole function of the DNA element is to disrupt the DNA sequence of a gene.

It is contemplated that unexpressed DNA sequences, including novel synthetic sequences could be introduced into cells as proprietary “labels” of those cells and plants and seeds thereof. It would not be necessary for a label DNA element to disrupt the function of a gene endogenous to the host organism, as the sole function of this DNA would be to identify the origin of the organism. For example, one could introduce a unique DNA sequence into a plant and this DNA element would identify all cells, plants, and progeny of these cells as having arisen from that labeled source. It is proposed that inclusion of label DNAs would enable one to distinguish proprietary germplasm or germplasm derived from such, from unlabelled germplasm.

Another possible element which may be introduced is a matrix attachment region element (MAR), such as the chicken lysozyme A element (Stief et al., 1989), which can be positioned around an expressible gene of interest to effect an increase in overall expression of the gene and diminish position dependant effects upon incorporation into the plant genome (Stief et al., 1989; Phi-Van et al., 1990).

III. Transformed (Transgenic) Plants of the Invention and Methods of Preparation

Plant species may be transformed with the DNA construct of the present invention by the DNA-mediated transformation of plant cell protoplasts and subsequent regeneration of the plant from the transformed protoplasts in accordance with procedures well known in the art.

Any plant tissue capable of subsequent clonal propagation, whether by organogenesis or embryogenesis, may be transformed with a vector of the present invention. The term “organogenesis,” as used herein, means a process by which shoots and roots are developed sequentially from meristematic centers; the term “embryogenesis,” as used herein, means a process by which shoots and roots develop together in a concerted fashion (not sequentially), whether from somatic cells or gametes. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristems, axillary buds, and root meristems), and induced meristem tissue (e.g., cotyledon meristem and ultilane meristem).

Plants of the present invention may take a variety of forms. The plants may be chimeras of transformed cells and non-transformed cells; the plants may be clonal transformants (e.g., all cells transformed to contain the expression cassette); the plants may comprise grafts of transformed and untransformed tissues (e.g., a transformed root stock grafted to an untransformed scion in citrus species). The transformed plants may be propagated by a variety of means, such as by clonal propagation or classical breeding techniques. For example, first generation (or T1) transformed plants may be selfed to give homozygous second generation (or T2) transformed plants, and the T2 plants further propagated through classical breeding techniques. A dominant selectable marker (such as npt II) can be associated with the expression cassette to assist in breeding.

Thus, the present invention provides a transformed (transgenic) plant cell, in planta or explanta, including a transformed plastid or other organelle, e.g., nucleus, mitochondria or chloroplast. The present invention may be used for transformation of any plant species, including, but not limited to, cells from corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassaya (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea ultilane), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, duckweed (Lemna), barley, vegetables, ornamentals, and conifers.

Duckweed (Lemna, see WO 00/07210) includes members of the family Lemnaceae. There are known four genera and 34 species of duckweed as follows: genus Lemna (L. aequinoctialis, L. disperma, L. ecuadoriensis, L. gibba, L. japonica, L. minor, L. miniscula, L. obscura, L. perpusilla, L. tenera, L. trisulca, L. turionifera, L. valdiviana); genus Spirodela (S. intermedia, S. polyrrhiza, S. punctata); genus Woffia (Wa. Angusta, Wa. Arrhiza, Wa. Australina, Wa. Borealis, Wa. Brasiliensis, Wa. Columbiana, Wa. Elongata, Wa. Globosa, Wa. Microscopica, Wa. Neglecta) and genus Wofiella (Wl. ultila, Wl. ultilanen, Wl. gladiata, Wl. ultila, Wl. lingulata, Wl. repunda, Wl. rotunda, and Wl. neotropica). Any other genera or species of Lemnaceae, if they exist, are also aspects of the present invention. Lemna gibba, Lemna minor, and Lemna miniscula are preferred, with Lemna minor and Lemna miniscula being most preferred. Lemna species can be classified using the taxonomic scheme described by Landolt, Biosystematic Investigation on the Family of Duckweeds: The family of Lemnaceae—A Monograph Study. Geobatanischen Institut ETH, Stiftung Rubel, Zurich (1986)).

Vegetables within the scope of the invention include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata), Douglas-fir (Pseudotsuga menziesii); Western hemlock (Tsuga ultilane); Sitka spruce (Picea glauca); redwood (Sequoia sempervirens); true firs such as silver fir (Abies amabilis) and balsam fir (Abies balsamea); and cedars such as Western red cedar (Thuja plicata) and Ak. yellow-cedar (Chamaecyparis nootkatensis). Leguminous plants include beans and peas. Beans include guar, locust bean, fenugreek, soybean, garden beans, cowpea, mungbean, lima bean, fava bean, lentils, chickpea, etc. Legumes include, but are not limited to, Arachis, e.g., peanuts, Vicia, e.g., crown vetch, hairy vetch, adzuki bean, mung bean, and chickpea, Lupinus, e.g., lupine, trifolium, Phaseolus, e.g., common bean and lima bean, Pisum, e.g., field bean, Melilotus, e.g., clover, Medicago, e.g., alfalfa, Lotus, e.g., trefoil, lens, e.g., lentil, and false indigo. Preferred forage and turf grass for use in the methods of the invention include alfalfa, orchard grass, tall fescue, perennial ryegrass, creeping bent grass, and redtop.

Papaya, garlic, pea, peach, pepper, petunia, strawberry, sorghum, sweet potato, turnip, safflower, corn, pea, endive, gourd, grape, snap bean, chicory, cotton, tobacco, aubergine, beet, buckwheat, broad bean, nectarine, avocado, mango, banana, groundnut, potato, peanut, lettuce, pineapple, spinach, squash, sugarbeet, sugarcane, sweet corn, chrysanthemum.

Other plants within the scope of the invention include Acacia, aneth, artichoke, arugula, blackberry, canola, cilantro, clementines, escarole, eucalyptus, fennel, grapefruit, honey dew, jicama, kiwifruit, lemon, lime, mushroom, nut, okra, orange, parsley, persimmon, plantain, pomegranate, poplar, radiata pine, radicchio, Southern pine, sweetgum, tangerine, triticale, vine, yams, apple, pear, quince, cherry, apricot, melon, hemp, buckwheat, grape, raspberry, chenopodium, blueberry, nectarine, peach, plum, strawberry, watermelon, eggplant, pepper, cauliflower, Brassica, e.g., broccoli, cabbage, ultilan sprouts, onion, carrot, leek, beet, broad bean, celery, radish, pumpkin, endive, gourd, garlic, snapbean, spinach, squash, turnip, ultilane, and zucchini.

Ornamental plants within the scope of the invention include impatiens, Begonia, Pelargonium, Viola, Cyclamen, Verbena, Vinca, Tagetes, Primula, Saint Paulia, Agertum, Amaranthus, Antihirrhinum, Aquilegia, Cineraria, Clover, Cosmo, Cowpea, Dahlia, Datura, Delphinium, Gerbera, Gladiolus, Gloxinia, Hippeastrum, Mesembryanthemum, Salpiglossos, and Zinnia. Other plants within the scope of the invention are shown in Table 1 (above).

Preferably, transgenic plants of the present invention are crop plants and in particular cereals (for example, corn, alfalfa, sunflower, rice, Brassica, canola, soybean, barley, soybean, sugarbeet, cotton, safflower, peanut, sorghum, wheat, millet, tobacco, etc.), and even more preferably corn, rice and soybean.

Transformation of plants can be undertaken with a single DNA molecule or multiple DNA molecules (i.e., co-transformation), and both these techniques are suitable for use with the expression cassettes of the present invention. Numerous transformation vectors are available for plant transformation, and the expression cassettes of this invention can be used in conjunction with any such vectors. The selection of vector will depend upon the preferred transformation technique and the target species for transformation.

A variety of techniques are available and known to those skilled in the art for introduction of constructs into a plant cell host. These techniques generally include transformation with DNA employing A. tumefaciens or A. rhizogenes as the transforming agent, liposomes, PEG precipitation, electroporation, DNA injection, direct DNA uptake, microprojectile bombardment, particle acceleration, and the like (See, for example, EP 295959 and EP 138341) (see below). However, cells other than plant cells may be transformed with the expression cassettes of the invention. The general descriptions of plant expression vectors and reporter genes, and Agrobacterium and Agrobacterium-mediated gene transfer, can be found in Gruber et al. (1993).

Expression vectors containing genomic or synthetic fragments can be introduced into protoplasts or into intact tissues or isolated cells. Preferably expression vectors are introduced into intact tissue. General methods of culturing plant tissues are provided for example by Maki et al., (1993); and by Phillips et al. (1988). Preferably, expression vectors are introduced into maize or other plant tissues using a direct gene transfer method such as microprojectile-mediated delivery, DNA injection, electroporation and the like. More preferably expression vectors are introduced into plant tissues using the microprojectile media delivery with the biolistic device. See, for example, Tomes et al. (1995). The vectors of the invention can not only be used for expression of structural genes but may also be used in exon-trap cloning, or promoter trap procedures to detect differential gene expression in varieties of tissues, (Lindsey et al., 1993; Auch & Reth et al.).

It is particularly preferred to use the binary type vectors of Ti and Ri plasmids of Agrobacterium spp. Ti-derived vectors transform a wide variety of higher plants, including monocotyledonous and dicotyledonous plants, such as soybean, cotton, rape, tobacco, and rice (Pacciotti et al., 1985: Byrne et al., 1987; Sukhapinda et al., 1987; Park et al., 1985: Hiei et al., 1994). The use of T-DNA to transform plant cells has received extensive study and is amply described (EP 120516; Hoekema, 1985; Knauf, et al., 1983; and An et al., 1985). For introduction into plants, the chimeric genes of the invention can be inserted into binary vectors as described in the examples.

Other transformation methods are available to those skilled in the art, such as direct uptake of foreign DNA constructs (see EP 295959), techniques of electroporation (Fromm et al., 1986) or high velocity ballistic bombardment with metal particles coated with the nucleic acid constructs (Kline et al., 1987, and U.S. Pat. No. 4,945,050). Once transformed, the cells can be regenerated by those skilled in the art. Of particular relevance are the recently described methods to transform foreign genes into commercially important crops, such as rapeseed (De Block et al., 1989), sunflower (Everett et al., 1987), soybean (McCabe et al., 1988; Hinchee et al., 1988; Chee et al., 1989; Christou et al., 1989; EP 301749), rice (Hie et al., 1994), and corn (Gordon Kamm et al., 1990; Fromm et al., 1990).

Those skilled in the art will appreciate that the choice of method might depend on the type of plant, i.e., monocotyledonous or dicotyledonous, targeted for transformation. Suitable methods of transforming plant cells include, but are not limited to, microinjection (Crossway et al., 1986), electroporation (Riggs et al., 1986), Agrobacterium-mediated transformation (Hinchee et al., 1988), direct gene transfer (Paszkowski et al., 1984), and ballistic particle acceleration using devices available from Agracetus, Inc., Madison, Wis. And BioRad, Hercules, Calif. (see, for example, Sanford et al., U.S. Pat. No. 4,945,050; and McCabe et al., 1988). Also see, Weissinger et al., 1988; Sanford et al., 1987 (onion); Christou et al., 1988 (soybean); McCabe et al., 1988 (soybean); Datta et al., 1990 (rice); Klein et al., 1988 (maize); Klein et al., 1988 (maize); Klein et al., 1988 (maize); Fromm et al., 1990 (maize); and Gordon-Kamm et al., 1990 (maize); Svab et al., 1990 (tobacco chloroplast); Koziel et al., 1993 (maize); Shimamoto et al., 1989 (rice); Christou et al., 1991 (rice); European Patent Application EP 0 332 581 (orchardgrass and other Pooideae); Vasil et al., 1993 (wheat); Weeks et al., 1993 (wheat). In one embodiment, the protoplast transformation method for maize is employed (European Patent Application EP 0 292 435, U.S. Pat. No. 5,350,689).

In another embodiment, a nucleotide sequence of the present invention is directly transformed into the plastid genome. Plastid transformation technology is extensively described in U.S. Pat. Nos. 5,451,513, 5,545,817, and 5,545,818, in PCT application no. WO 95/16783, and in McBride et al., 1994. The basic technique for chloroplast transformation involves introducing regions of cloned plastid DNA flanking a selectable marker together with the gene of interest into a suitable target tissue, e.g., using biolistics or protoplast transformation (e.g., calcium chloride or PEG mediated transformation). The 1 to 1.5 kb flanking regions, termed targeting sequences, facilitate orthologous recombination with the plastid genome and thus allow the replacement or modification of specific regions of the plastome. Initially, point mutations in the chloroplast 16S rRNA and rps12 genes conferring resistance to spectinomycin and/or streptomycin are utilized as selectable markers for transformation (Svab et al., 1990; Staub et al., 1992). This resulted in stable homoplasmic transformants at a frequency of approximately one per 100 bombardments of target leaves. The presence of cloning sites between these markers allowed creation of a plastid targeting vector for introduction of foreign genes (Staub et al., 1993). Substantial increases in transformation frequency are obtained by replacement of the recessive rRNA or r-protein antibiotic resistance genes with a dominant selectable marker, the bacterial aadA gene encoding the spectinomycin-detoxifying enzyme aminoglycoside-3′-adenyltransferase (Svab et al., 1993). Other selectable markers useful for plastid transformation are known in the art and encompassed within the scope of the invention. Typically, approximately 15-20 cell division cycles following transformation are required to reach a homoplastidic state. Plastid expression, in which genes are inserted by orthologous recombination into all of the several thousand copies of the circular plastid genome present in each plant cell, takes advantage of the enormous copy number advantage over nuclear-expressed genes to permit expression levels that can readily exceed 10% of the total soluble plant protein. In a preferred embodiment, a nucleotide sequence of the present invention is inserted into a plastid targeting vector and transformed into the plastid genome of a desired plant host. Plants homoplastic for plastid genomes containing a nucleotide sequence of the present invention are obtained, and are preferentially capable of high expression of the nucleotide sequence.

Agrobacterium tumefaciens cells containing a vector comprising an expression cassette of the present invention, wherein the vector comprises a Ti plasmid, are useful in methods of making transformed plants. Plant cells are infected with an Agrobacterium tumefaciens as described above to produce a transformed plant cell, and then a plant is regenerated from the transformed plant cell. Numerous Agrobacterium vector systems useful in carrying out the present invention are known.

For example, vectors are available for transformation using Agrobacterium tumefaciens. These typically carry at least one T-DNA border sequence and include vectors such as pBIN19 (Bevan, 1984). In one preferred embodiment, the expression cassettes of the present invention may be inserted into either of the binary vectors pCIB200 and pCIB2001 for use with Agrobacterium. These vector cassettes for Agrobacterium-mediated transformation wear constructed in the following manner. PTJS75kan was created by NarI digestion of pTJS75 (Schmidhauser & Helinski, 1985) allowing excision of the tetracycline-resistance gene, followed by insertion of an AccI fragment from pUC4K carrying an NPTII (Messing & Vierra, 1982; Bevan et al., 1983; McBride et al., 1990). XhoI linkers were ligated to the EcoRV fragment of pCIB7 which contains the left and right T-DNA borders, a plant selectable nos/nptII chimeric gene and the pUC polylinker (Rothstein et al., 1987), and the XhoI-digested fragment was cloned into SalI-digested pTJS75kan to create pCIB200 (see also EP 0 332 104, example 19). PCIB200 contains the following unique polylinker restriction sites: EcoRI, SstI, KpnI, BglII, XbaI, and SalI. The plasmid pCIB2001 is a derivative of pCIB200 which was created by the insertion into the polylinker of additional restriction sites. Unique restriction sites in the polylinker of pCIB2001 are EcoRI, SstI, KpnI, BglII, XbaI, SalI, MluI, BclI, AvrII, ApaI, HpaI, and StuI. PCIB2001, in addition to containing these unique restriction sites also has plant and bacterial kanamycin selection, left and right T-DNA borders for Agrobacterium-mediated transformation, the RK2-derived trfA function for mobilization between E. coli and other hosts, and the OriT and OriV functions also from RK2. The pCIB2001 polylinker is suitable for the cloning of plant expression cassettes containing their own regulatory signals.

An additional vector useful for Agrobacterium-mediated transformation is the binary vector pCIB10, which contains a gene encoding kanamycin resistance for selection in plants, T-DNA right and left border sequences and incorporates sequences from the wide host-range plasmid pRK252 allowing it to replicate in both E. coli and Agrobacterium. Its construction is described by Rothstein et al., 1987. Various derivatives of pCIB10 have been constructed which incorporate the gene for hygromycin B phosphotransferase described by Gritz et al., 1983. These derivatives enable selection of transgenic plant cells on hygromycin only (pCIB743), or hygromycin and kanamycin (pCIB715, pCIB717).

Methods using either a form of direct gene transfer or Agrobacterium-mediated transfer usually, but not necessarily, are undertaken with a selectable marker which may provide resistance to an antibiotic (e.g., kanamycin, hygromycin or methotrexate) or a herbicide (e.g., phosphinothricin). The choice of selectable marker for plant transformation is not, however, critical to the invention.

For certain plant species, different antibiotic or herbicide selection markers may be preferred. Selection markers used routinely in transformation include the nptII gene which confers resistance to kanamycin and related antibiotics (Messing & Vierra, 1982; Bevan et al., 1983), the bar gene which confers resistance to the herbicide phosphinothricin (White et al., 1990, Spencer et al., 1990), the hph gene which confers resistance to the antibiotic hygromycin (Blochinger & Diggelmann), and the dhfr gene, which confers resistance to methotrexate (Bourouis et al., 1983).

One such vector useful for direct gene transfer techniques in combination with selection by the herbicide Basta (or phosphinothricin) is pCIB3064. This vector is based on the plasmid pCIB246, which comprises the CaMV 35S promoter in operational fusion to the E. coli GUS gene and the CaMV 35S transcriptional terminator and is described in the PCT published application WO 93/07278, herein incorporated by reference. One gene useful for conferring resistance to phosphinothricin is the bar gene from Streptomyces viridochromogenes (Thompson et al., 1987). This vector is suitable for the cloning of plant expression cassettes containing their own regulatory signals.

An additional transformation vector is pSOG35 which utilizes the E. coli gene dihydrofolate reductase (DHFR) as a selectable marker conferring resistance to methotrexate. PCR was used to amplify the 35S promoter (about 800 bp), intron 6 from the maize Adh1 gene (about 550 bp) and 18 bp of the GUS untranslated leader sequence from pSOG10. A 250 bp fragment encoding the E. coli dihydrofolate reductase type II gene was also amplified by PCR and these two PCR fragments were assembled with a SacI-PstI fragment from pBI221 (Clontech) which comprised the pUC19 vector backbone and the nopaline synthase terminator. Assembly of these fragments generated pSOG19 which contains the 35S promoter in fusion with the intron 6 sequence, the GUS leader, the DHFR gene and the nopaline synthase terminator. Replacement of the GUS leader in pSOG19 with the leader sequence from Maize Chlorotic Mottle Virus check (MCMV) generated the vector pSOG35. pSOG19 and pSOG35 carry the pUC-derived gene for ampicillin resistance and have HindIII, SphI, PstI and EcoRI sites available for the cloning of foreign sequences.

IV. Production and Characterization of Stably Transformed Plants

Transgenic plant cells are then placed in an appropriate selective medium for selection of transgenic cells which are then grown to callus. Shoots are grown from callus and plantlets generated from the shoot by growing in rooting medium. The various constructs normally will be joined to a marker for selection in plant cells. Conveniently, the marker may be resistance to a biocide (particularly an antibiotic, such as kanamycin, G418, bleomycin, hygromycin, chloramphenicol, herbicide, or the like). The particular marker used will allow for selection of transformed cells as compared to cells lacking the DNA which has been introduced. Components of DNA constructs including transcription cassettes of this invention may be prepared from sequences which are native (endogenous) or foreign (exogenous) to the host. By “foreign” it is meant that the sequence is not found in the wild-type host into which the construct is introduced. Heterologous constructs will contain at least one region which is not native to the gene from which the transcription-initiation-region is derived.

To confirm the presence of the transgenes in transgenic cells and plants, a variety of assays may be performed. Such assays include, for example, “molecular biological” assays well known to those of skill in the art, such as Southern and Northern blotting, in situ hybridization and nucleic acid-based amplification methods such as PCR or RT-PCR; “biochemical” assays, such as detecting the presence of a protein product, e.g., by immunological means (ELISAs and Western blots) or by enzymatic function; plant part assays, such as leaf or root assays; and also, by analyzing the phenotype of the whole regenerated plant, e.g., for disease or pest resistance.

DNA may be isolated from cell lines or any plant parts to determine the presence of the preselected nucleic acid segment through the use of techniques well known to those skilled in the art. Note that intact sequences will not always be present, presumably due to rearrangement or deletion of sequences in the cell.

The presence of nucleic acid elements introduced through the methods of this invention may be determined by polymerase chain reaction (PCR). Using this technique discreet fragments of nucleic acid are amplified and detected by gel electrophoresis. This type of analysis permits one to determine whether a preselected nucleic acid segment is present in a stable transformant, but does not prove integration of the introduced preselected nucleic acid segment into the host cell genome. In addition, it is not possible using PCR techniques to determine whether transformants have exogenous genes introduced into different sites in the genome, i.e., whether transformants are of independent origin. It is contemplated that using PCR techniques it would be possible to clone fragments of the host genomic DNA adjacent to an introduced preselected DNA segment.

Positive proof of DNA integration into the host genome and the independent identities of transformants may be determined using the technique of Southern hybridization. Using this technique specific DNA sequences that were introduced into the host genome and flanking host DNA sequences can be identified. Hence the Southern hybridization pattern of a given transformant serves as an identifying characteristic of that transformant. In addition it is possible through Southern hybridization to demonstrate the presence of introduced preselected DNA segments in high molecular weight DNA, i.e., confirm that the introduced preselected DNA segment has been integrated into the host cell genome. The technique of Southern hybridization provides information that is obtained using PCR, e.g., the presence of a preselected DNA segment, but also demonstrates integration into the genome and characterizes each individual transformant.

It is contemplated that using the techniques of dot or slot blot hybridization which are modifications of Southern hybridization techniques one could obtain the same information that is derived from PCR, e.g., the presence of a preselected DNA segment.

Both PCR and Southern hybridization techniques can be used to demonstrate transmission of a preselected DNA segment to progeny. In most instances the characteristic Southern hybridization pattern for a given transformant will segregate in progeny as one or more Mendelian genes (Spencer et al., 1992); Laursen et al., 1994) indicating stable inheritance of the gene. The nonchimeric nature of the callus and the parental transformants (R₀) was suggested by germline transmission and the identical Southern blot hybridization patterns and intensities of the transforming DNA in callus, R₀ plants and R₁ progeny that segregated for the transformed gene.

Whereas DNA analysis techniques may be conducted using DNA isolated from any part of a plant, RNA may only be expressed in particular cells or tissue types and hence it will be necessary to prepare RNA for analysis from these tissues. PCR techniques may also be used for detection and quantitation of RNA produced from introduced preselected DNA segments. In this application of PCR it is first necessary to reverse transcribe RNA into DNA, using enzymes such as reverse transcriptase, and then through the use of conventional PCR techniques amplify the DNA. In most instances PCR techniques, while useful, will not demonstrate integrity of the RNA product. Further information about the nature of the RNA product may be obtained by Northern blotting. This technique will demonstrate the presence of an RNA species and give information about the integrity of that RNA. The presence or absence of an RNA species can also be determined using dot or slot blot Northern hybridizations. These techniques are modifications of Northern blotting and will only demonstrate the presence or absence of an RNA species.

While Southern blotting and PCR may be used to detect the preselected DNA segment in question, they do not provide information as to whether the preselected DNA segment is being expressed. Expression may be evaluated by specifically identifying the protein products of the introduced preselected DNA segments or evaluating the phenotypic changes brought about by their expression.

Assays for the production and identification of specific proteins may make use of physical-chemical, structural, functional, or other properties of the proteins. Unique physical-chemical or structural properties allow the proteins to be separated and identified by electrophoretic procedures, such as native or denaturing gel electrophoresis or isoelectric focusing, or by chromatographic techniques such as ion exchange or gel exclusion chromatography. The unique structures of individual proteins offer opportunities for use of specific antibodies to detect their presence in formats such as an ELISA assay. Combinations of approaches may be employed with even greater specificity such as Western blotting in which antibodies are used to locate individual gene products that have been separated by electrophoretic techniques. Additional techniques may be employed to absolutely confirm the identity of the product of interest such as evaluation by amino acid sequencing following purification. Although these are among the most commonly employed, other procedures may be additionally used.

Assay procedures may also be used to identify the expression of proteins by their functionality, especially the ability of enzymes to catalyze specific chemical reactions involving specific substrates and products. These reactions may be followed by providing and quantifying the loss of substrates or the generation of products of the reactions by physical or chemical procedures. Examples are as varied as the enzyme to be analyzed.

Very frequently the expression of a gene product is determined by evaluating the phenotypic results of its expression. These assays also may take many forms including but not limited to analyzing changes in the chemical composition, morphology, or physiological properties of the plant. Morphological changes may include greater stature or thicker stalks. Most often changes in response of plants or plant parts to imposed treatments are evaluated under carefully controlled conditions termed bioassays.

V. Uses of Transgenic Plants

Once an expression cassette of the invention has been transformed into a particular plant species, it may be propagated in that species or moved into other varieties of the same species, particularly including commercial varieties, using traditional breeding techniques. Particularly preferred plants of the invention include the agronomically important crops listed above. The genetic properties engineered into the transgenic seeds and plants described above are passed on by sexual reproduction and can thus be maintained and propagated in progeny plants. The present invention also relates to a transgenic plant cell, tissue, organ, seed or plant part obtained from the transgenic plant. Also included within the invention are transgenic descendants of the plant as well as transgenic plant cells, tissues, organs, seeds and plant parts obtained from the descendants.

Preferably, the expression cassette in the transgenic plant is sexually transmitted. In one preferred embodiment, the coding sequence is sexually transmitted through a complete normal sexual cycle of the R0 plant to the R1 generation. Additionally preferred, the expression cassette is expressed in the cells, tissues, seeds or plant of a transgenic plant in an amount that is different than the amount in the cells, tissues, seeds or plant of a plant which only differs in that the expression cassette is absent.

The transgenic plants produced herein are thus expected to be useful for a variety of commercial and research purposes. Transgenic plants can be created for use in traditional agriculture to possess traits beneficial to the grower (e.g., agronomic traits such as resistance to water deficit, pest resistance, herbicide resistance or increased yield), beneficial to the consumer of the grain harvested from the plant (e.g., improved nutritive content in human food or animal feed; increased vitamin, amino acid, and antioxidant content; the production of antibodies (passive immunization) and nutriceuticals), or beneficial to the food processor (e.g., improved processing traits). In such uses, the plants are generally grown for the use of their grain in human or animal foods. Additionally, the use of root-specific promoters in transgenic plants can provide beneficial traits that are localized in the consumable (by animals and humans) roots of plants such as carrots, parsnips, and beets. However, other parts of the plants, including stalks, husks, vegetative parts, and the like, may also have utility, including use as part of animal silage or for ornamental purposes. Often, chemical constituents (e.g., oils or starches) of maize and other crops are extracted for foods or industrial use and transgenic plants may be created which have enhanced or modified levels of such components.

Transgenic plants may also find use in the commercial manufacture of proteins or other molecules, where the molecule of interest is extracted or purified from plant parts, seeds, and the like. Cells or tissue from the plants may also be cultured, grown in vitro, or fermented to manufacture such molecules.

The transgenic plants may also be used in commercial breeding programs, or may be crossed or bred to plants of related crop species. Improvements encoded by the expression cassette may be transferred, e.g., from maize cells to cells of other species, e.g., by protoplast fusion.

The transgenic plants may have many uses in research or breeding, including creation of new mutant plants through insertional mutagenesis, in order to identify beneficial mutants that might later be created by traditional mutation and selection. An example would be the introduction of a recombinant DNA sequence encoding a transposable element that may be used for generating genetic variation. The methods of the invention may also be used to create plants having unique “signature sequences” or other marker sequences which can be used to identify proprietary lines or varieties.

Thus, the transgenic plants and seeds according to the invention can be used in plant breeding which aims at the development of plants with improved properties conferred by the expression cassette, such as tolerance of drought, disease, or other stresses. The various breeding steps are characterized by well-defined human intervention such as selecting the lines to be crossed, directing pollination of the parental lines, or selecting appropriate descendant plants. Depending on the desired properties different breeding measures are taken. The relevant techniques are well known in the art and include but are not limited to hybridization, inbreeding, backcross breeding, ultilane breeding, variety blend, interspecific hybridization, aneuploid techniques, etc. Hybridization techniques also include the sterilization of plants to yield male or female sterile plants by mechanical, chemical or biochemical means. Cross pollination of a male sterile plant with pollen of a different line assures that the genome of the male sterile but female fertile plant will uniformly obtain properties of both parental lines. Thus, the transgenic seeds and plants according to the invention can be used for the breeding of improved plant lines which for example increase the effectiveness of conventional methods such as herbicide or pesticide treatment or allow to dispense with said methods due to their modified genetic properties. Alternatively new crops with improved stress tolerance can be obtained which, due to their optimized genetic “equipment”, yield harvested product of better quality than products which were not able to tolerate comparable adverse developmental conditions.

VI. A Computer Readable Medium

The invention also provides a computer readable medium having stored thereon a data structure containing nucleic acid sequences having at least 70% sequence identity to a nucleic acid sequence selected from those listed in SEQ ID Nos: 1-953, 1001-1095, 1954-1966, 2000-2129, 2137-2661, 2662-4737 and 4738-6813, as well as complementary, ortholog, and variant sequences thereof. Storage and use of nucleic acid sequences on a computer readable medium is well known in the art. (See for example U.S. Pat. Nos. 6,023,659; 5,867,402; 5,795,716) Examples of such medium include, but are not limited to, magnetic tape, optical disk, CD-ROM, random access memory, volatile memory, non-volatile memory and bubble memory. Accordingly, the nucleic acid sequences contained on the computer readable medium may be compared through use of a module that receives the sequence information and compares it to other sequence information. Examples of other sequences to which the nucleic acid sequences of the invention may be compared include those maintained by the National Center for Biotechnology Information (NCBI)(http://www.ncbi.nlm.nih.gov/) and the Swiss Protein Data Bank. A computer is an example of such a module that can read and compare nucleic acid sequence information. Accordingly, the invention also provides the method of comparing a nucleic acid sequence of the invention to another sequence. For example, a sequence of the invention may be submitted to the NCBI for a Blast search as described herein where the sequence is compared to sequence information contained within the NCBI database and a comparison is returned. The invention also provides nucleic acid sequence information in a computer readable medium that allows the encoded polypeptide to be optimized for a desired property. Examples of such properties include, but are not limited to, increased or decreased: thermal stability, chemical stability, hydrophylicity, hydrophobicity, and the like. Methods for the use of computers to model polypeptides and polynucleotides having altered activities are well known in the art and have been reviewed. (Lesyng et al., 1993; Surles et al., 1994; Koehl et al., 1996; Rossi et al., 2001).

The invention will be further described by the following non-limiting examples.

### EXAMPLE 1

### GeneChip Standard Protocol

Quantitation of Total RNA

Total RNA from plant tissue is extracted and quantified.

1. Quantify total RNA using GeneQuant
1OD₂₆₀=40 mg RNA/ml; A260/A280=1.9 to about 2.1

2. Run gel to check the integrity and purity of the extracted RNA

Synthesis of Double-Stranded cDNA

Gibco/BRL SuperScript Choice System for cDNA Synthesis (Cat#1B090-019) was employed to prepare cDNAs. T7-(dT)₂₄ oligonucleotides were prepared and purified by HPLC. (5′-GGCCAGTGAATTGTAATACGACTCACTATAGGGAGGCGG-(dT)₂₄-3′ SEQ ID NO:2136).

Step 1. Primer hybridization:
Incubate at 70° C. for 10 minutes
Quick spin and put on ice briefly

Step 2. Temperature adjustment:
Incubate at 42° C. for 2 minutes

Step 3. First strand synthesis:
DEPC-water-1 μl
RNA (101 g final)-10 μl
T7=(dT)₂₄ Primer (100 pmol final)-1 μl pmol
5× 1st strand cDNA buffer-4 μl
0.1M DTT (10 mM final)-2 μl
10 mM dNTP mix (500 μM final)-1 μl
Superscript II RT 200 U/μl-1 μl
Total of 20 μl
Mix well
Incubate at 42° C. for 1 hour

Step 4. Second strand synthesis:
Place reactions on ice, quick spin
DEPC-water—91 μl
5× 2nd strand cDNA buffer-30 μl mM dNTP mix (250 mM final)—3 μl
E. coli DNA ligase (10 U/μl)—1 μl
E. coli DNA polymerase 1-10 U/μl-4 μl
RnaseH 2 U/μl—1 μl
T4 DNA polymerase 5 U/μl—2 μl
0.5 M EDTA (0.5 M final)—10 μl
Total 162 μl
Mix/spin down/incubate 16° C. for 2 hours

Step 5. Completing the reaction:
Incubate at 16° C. for 5 minutes
   Purification of Double Stranded cDNA
1. Centrifuge PLG (Phase Lock Gel, Eppendorf 5 Prime, Inc., PI-188233) at 14,000×, transfer 162 μl of cDNA to PLG
2. Add 162 μl of Phenol:Chloroform:Isoamyl alcohol (pH 8.0), centrifuge 2 minutes

3. Transfer the supernatant to a fresh 1.5 ml tube, add

| | |
|---|---|
| Glycogen (5 mg/ml) | 2 |
| 0.5 M NH₄OAC (0.75 × Vol) | 120 |
| ETOH (2.5 × Vol, −20 C.) | 400 |

4. Mix well and centrifuge at 14,000× for 20 minutes
5. Remove supernatant, add 0.5 ml 80% EtOH (−20° C.)
6. Centrifuge for 5 minutes, air dry or by speed vac for 5-10 minutes
7. Add 44 μl DEPC H₂O
   Analyze of quantity and size distribution of cDNA
   Run a gel using 1 μl of the double-stranded synthesis product
   Synthesis of Biotinylated cRNA

(use Enzo BioArray High Yield RNA Transcript Labeling Kit Cat#900182)

| | |
|---|---|
| Purified cDNA | 22 μl |
| 10X Hy buffer | 4 μl |
| 10X biotin ribonucleotides | 4 μl |
| 10X DTT | 4 μl |
| 10X Rnase inhibitor mix | 4 μl |
| 20X T7 RNA polymerase | 2 μl |
| Total | 40 μl |

Centrifuge 5 seconds, and incubate for 4 hours at 37° C.

Gently mix every 30-45 minutes

Purification and Quantification of cRNA

(use Qiagen Rneasy Mini kit Cat# 74103)

Determine concentration and dilute to 1 μg/μl concentration

Fragmentation of cRNA

| | |
|---|---|
| cRNA (1 μg/μl) | 15 μl |
| 5X Fragmentation Buffer* | 6 μl |
| DEPC H₂O | 9 μl |
| | 30 μl |

*5x Fragmentation Buffer

| | |
|---|---|
| 1M Tris (pH8.1) | 4.0 ml |
| MgOAc | 0.64 g |
| KOAC | 0.98 g |
| DEPC H₂O | |
| Total | 20 ml |

Filter Sterilize

Array Wash and Staining

Stringent Wash Buffer**

Non-Stringent Wash Buffer***

SAPE Stain****

Antibody Stain*****

Wash on fluidics station using the appropriate antibody amplification protocol

**Stringent Buffer: 12×MES 83.3 ml, 5 M NaCl 5.2 ml, 10% Tween 1.0 ml, H₂O 910 ml,
   Filter Sterilize
***Non-Stringent Buffer: 20× SSPE 300 ml, 10% Tween 1.0 ml, H₂O 698 ml, Filter Sterilize, Antifoam 1.0.
****SAPE stain: 2× Stain Buffer 600 μl, BSA 48 μl, SAPE 12 μl, H₂O 540 μl.
*****Antibody Stain: 2× Stain Buffer 300 μl, H₂O 266.4 μl, BSA 24 μl, Goat IgG 6 μl, Biotinylated Ab 3.6 μl
   Image Analysis and Data Mining
   1. Two text files are included in the analysis:
   a. One with Absolute analysis: giving the status of each gene, either absent or present in the samples
   b. The other with Comparison analysis: comparing gene expression levels between two samples

### EXAMPLE 2

### Analysis of the RPS2 Mediated Interaction in Arabidopsis

The identification and cloning of resistance genes is extremely important for the treatment of crops. For example, bacterial blight disease caused by Xanthomonas spp. infects virtually all crop plants and leads to extensive crop losses worldwide. Therefore, it is of interest to identify diverse and abundant plant resistance genes for use as future crop treatments for pathogen resistance, e.g., to identify particular pathogen resistance (R) genes in a plant.

Differential gene expression analysis was used to identify pathogen resistance (R) genes in a plant. This method takes advantage of the HR-associated disease resistance. One model plant-pathogen interaction is that of Arabidopsis thaliana and Pseudomonas syringae pv tomato. There are four possible genetic interactions of a P. syringae infection of Arabidopsis when analyzing HR-associated disease resistance (Table 2). However, there are only two possible outcomes: a compatible outcome occurs when there is disease, and an incompatible outcome occurs when there is no disease. An incompatible outcome, or disease resistance, occurs only when the plant possesses the resistance gene, e.g., RPS2, and the pathogen posesses the corresponding avr gene, e.g., avrRpt2. RPS2 belongs to the NBS-LRR class of R genes, which can confer resistance to a wide variety of phytopathogens. It has been suggested that AvrRpt2 is delivered to the plant via the bacteria's type III secretion system and recognized by a surveillance system involving RPS2 inside the plant cell. The plant response during an incompatible interaction includes a change in ion flux across the plasma membrane, generation of reactive oxygen species, induction of defense genes, induction of HR, fortification of the cell wall, accumulation of salicylic acid, and anti-microbial compounds.

TABLE 2

| | | | |
|---|---|---|---|
| Number | Plant | Pathogen | Outcome |

| | | | | |
|---|---|---|---|---|
| 1 | RPS2 | no avr | Disease | Compatible |
| 2 | RPS2 | avrRpt2 | No disease | Incompatible |
| 3 | rps2 | no avr | Disease | Compatible |
| 4 | rps2 | avrRpt2 | Disease | Compatible |

Methods

Differential Expression

Analysis of differential gene expression is a classic and very powerful tool in experimental biology not only to study large trends in gene regulation but also small differences among similar responses. Historically, methods for analysis only allowed the comparison of a very few genes in each experiment. However, with new methods to identify and quantitate differential mRNA profiles, such as long distance differential display PCR, cDNA microarrays, and gene chips, one can much more quickly and comprehensively identify and analyze differentially expressed genes.

By analyzing and comparing the expression profile of genes in the above 4-way matrix, a number of types of genes can be identified that are involved in the resistance pathway. Resistance genes would be highly expressed or strongly downregulated in outcome number 2 in the four way matrix and less oppositely expressed in outcome numbers 1, 3, and 4. Genes that are highly expressed or strongly downregulated in outcome numbers 1 and 2 and oppositely expressed or not expressed above baseline in outcome numbers 3 and 4 are of interest as being associated with the reaction of a plant having resistance genes to a bacterial infection, regardless of the avr genotype of the bacterium. Such a comparison is very useful in identifying strong candidates for different roles in plant/pathogen interactions, as are numerous other kinds of outcomes in the four-way plant/pathogen interaction analysis of gene expression. Such genes include those involved in recognition of pathogen (unrelated to virulence status); genes involved in recognition of pathogen having a virulence or avirulence gene (regardless of the status of the corresponding plant); genes related to the status of the plant, regardless of the status of the pathogen; and genes that do not change expression during plant-pathogen interaction.

Use of a Gene Chip to Study Gene Regulation in Arabidopsis in Response to Exposure to Pathogen

Initially isogenic strains of Arabidopsis thaliana ecotype Col-0 were used, one having the wild type RPS2 gene that confers resistance, and one having the rps2 mutant that confers susceptibility to attack by Pseudomonas syringae pathovar tomato (Pst). Subsequently, comparisons between ecotypes, mutant Arabidopsis, and infection with different pathogens were made. After infection, the RNA was isolated and a probe produced using the Affymetrix GeneChip™ protocol. A gene array representing approximately 8,100 Arabidopsis thaliana genes was used to carry out global gene expression profiling in response to exposure to a particular pathogen.

Initially, the analysis involved comparing all four of the interactions to a water control (plants “infected” with water). In the initial analysis, the mRNA levels of approximately 1,600 genes were significantly affected (>2.5-fold change in expression) by exposure to the bacterial pathogen. This suggested a dramatic change in the molecular biology of the cell and a more detailed analysis was performed.

Results

A. Comparison of Compatible To Incompatible Infections

Two different types of interactions between Arabidopsis and Pseudomonas syringae were analyzed. In one type of experiment, a gene for gene interaction conditioned by the plant resistance (R) gene RPS2 and the bacterial avirulence gene avrRpt2 at a relatively early stage was analyzed. When the pathogen has an avr gene and the plant has the corresponding R gene, the plant is resistant to the pathogen and the interaction is called incompatible. When the plant-pathogen system lacks either or both genes, the plant is susceptible to the pathogen and the interaction is called compatible. A hypersensitive response (HR, localized rapid cell death of the plant) is one aspect of resistance.

Isogenic strains of Arabidopsis thaliana ecotype Col-0 were used, one having the wild type RPS2 gene that confers resistance, and one having the mutant rps2 mutant that confers susceptibility to attack by Pseudomonas syringae pathovar tomato (Pst) carrying avrRpt2. Two strains of Pseudomonas syringae were used, one having the avr gene avrRpt2 and the other having no avr. The avr gene is carried on a plasmid.

A gene array having 8,700 probe sets representing approximately 8,100 Arabidopsis thaliana genes was used to carry out global gene expression profiling of each of the infection outcomes. The pairings were as follows:

1. RPS2 WT plant; P. syringae (no avr)

2. RPS2 WT plant; P. syringae/avrRpt2

3. rps2-101C mutant plant; P. syringae (no avr)

4. rps2-101C mutant plant; P. syringae/avrRpt2

Additionally, two controls were used:

5. RPS2 WT plant; water control

6. Rps2-101C mutant plant; water control

Data were processed such that genes having a difference in mRNA levels that was greater than 2.5-fold increased or reduced, compared with controls were selected. The fold change for each gene was log-scaled and normalized.

1. Data Analysis: Identification of Expression Clusters

Data analysis was carried out by comparing expression of each gene in interactions 1-4 (Table 2), plotting that expression level, and identifying the genes of interest, i.e., those that show more than a 2.5× change in expression (about 1,600 genes). Classification of patterns, or expression clusters were as follows:

a) Genes strongly induced (>2.5× change in expression level) only in the resistant (incompatible) response;

b) Genes responding weakly only in the resistance response, but strongly induced in the compatible response;

c) Genes that show a high level of expression in all outcomes;

d) Genes that show a high level of repression in all outcomes;

e) Genes that show a very high level of repression only when the bacterial avr is expressed; and

f) Genes that show a very different level of expression in the presence of the plant resistance compared to the level in the absence of the plant resistance (the mutant rps2).

Genes that fall within groups 1a and 1b, i.e., those that are differentially expressed only when an incompatible interaction occurs, include genes directly involved in resistance to pathogens. These genes show a peak (either up or down) only during plant-pathogen interaction 2. The differential expression can be of two types: upregulated (increased expression of this gene is potentially important in the incompatible interaction) or downregulated (decreased expression of this gene is potentially important in the incompatible interaction).

2. Heat Shock Proteins and Transcription Factors

All major heat shock proteins (HSPs) were identified to be upregulated only during the incompatible interaction. Heat shock factors (HSFs) are transcription factors which control the transcription of the HSP genes. Eight HSF genes are known in Arabidopsis. HSF4 and HSF21 were identified as being upregulated when the plant was infected with P. syringae. HSF4 showed strong induction that was restricted to resistance, and HSF4 was the only HSF specifically upregulated during the incompatible interaction. The data suggests that the upregulation of HSPs is downstream of upregulation of HSF4.

To analyze whether the response was a more general one, or specific to a given ecotype, expression of HSF4 was analysed in two different Arabidopsis ecotypes, A. thaliana, ecotypes Col-0 and Ws. HSF4 was also upregulated in the response of Ws ecotype to infection and, specifically, was upregulated during an incompatible response. HSF21 is thus a preferred protein for resistance applications, and HSF4, a protein which is expressed in all plants, is especially preferred for engineering resistance.

A transgene containing the ACT2 promoter and the HSF4 open reading frame was introduced to Arabidopsis and transgenic HSF4 Arabidopsis lines generated to overexpress and underexpress HSF4. The expression of HSF4 during pathogen infection may cause lower general resistance to P. syringae.

Conditional overexpression lines were also generated using the estradiol-inducible promoter system. Infiltration of 20 μM estradiol into the intercellular space of the leaves of transgenic plants induced expression of HSF4 mRNA for a short time (down by 4 hours). Addition of 20 estradiol to the hydroponic medium yielded sustained HSF4 mRNA accumulation.

B. Genes Involved in Arabidopsis Responses to Pathogens

A number of mutations in Arabidopsis thaliana that disrupt expression of pathogen-induced genes and cause enhanced disease susceptibility have been identified. Pathogen-induced genes whose expression is altered in these enhanced disease susceptibility mutants are likely to play important roles in conferring disease resistance.

To identify such genes, wild type and various mutant plants were infected with strain Psm ES4326 at a dose of 10,000 colony forming units per square centimeter of leaf tissue. Control plants were mock-infected. After thirty hours, tissue samples were collected and used to prepare RNA. Three sets of experiments were carried out. Each set of experiments included three independent replicate experiments. RNA from replicate experiments was pooled to reduce errors arising from the effects of variations in environmental conditions. Each RNA sample was used to prepare a fluorescently-labelled probe which was applied to an Affymetrix GeneChip™, allowing the expression level of each gene represented on the GeneChip™ to be determined for each sample. The plant genotypes included in each experiment were as follows:

Experiment #1

Wild-type (ecotype Columbia)

nahG

pad4-1

eds5-1

eds4

pad2-1

npr1-1

npr1-3

Experiment #2

Wild-type (ecotype Columbia)

coi1

ein2

pad1

FN1-3

eds3

eds8

Experiment #3

Wild-type (ecotype Columbia)

pad4-1

nahG

sid2

eds5-3

FN1-9

FN3-2

1. Data Analysis

Expression values that were less than 5 were set to five. This ensures that no values are 0 or negative. Such values interfere with subsequent analysis steps. To obtain a list of pathogen-induced genes, the ratios of infected wild-type to mock infected wild type were calculated for each experiment. Then genes were selected in which expression levels were infected wild-type/mock wild-type >2.5, and infected wild-type >50 for at least 2 of 3 experiments. The ratio of 2.5 was chosen because the false positive rate for the GeneChip™ is essentially 0 at this level of stringency, and the absolute value of 50 was chosen to eliminate expression values below the detection limit of the GeneChip™. The result of this analysis was a list of 745 probe sets representing genes that are induced by infection in wild-type plants (note that some genes are represented by more than one probe set, so the number of different genes is somewhat fewer) (see Table 3 below). Hence, the expression of genes comprising SEQ ID NOs:2-6, 16, 18, 22-23, 25, 28-29, 31-32, 35-37, 39-43, 45-47, 49-50, 52, 54-55, 57-58, 60-66, 70-72, 74, 76-77, 79, 81, 83, 85, 87-90, 92, 94, 97, 100-107, 111-115, 117-125, 127-135, 138-140, 142-153, 156-158, 160, 162-165, 168-170, 173-181, 183-184, 186-188, 190-198, 200-201, 203-211, 214-215, 218-224, 227-232, 234-249, 251-262, 264, 266-268, 270, 272-275, 277-281, 283, 286-294, 297-298, 302, 304-306, 308-326, 328-339, 341, 344-345, 347, 350-351, 353-358, 361-371, 373-377, 379-386, 388-390, 392, 394-400, 402-406, 408-410, 412-417, 419-427, 429-433, 435-443, 445-452, 454-457, 459-460, 462-464, 466-470, 473-475, 478-479, 481-482, 484-187, 489-494, 496-498, 500-501, 503-506, 508, 510, 512-515, 517-523, 526, 528-529, 531-538, 540, 544-548, 550-558, 560, 563-568, 570, 572-577, 579-580, 582-585, 588-594, 596, 598-600, 602-603, 605-606, 608-612, 614-617, 619-624, 626-630, 632-639, 642, 644, 646-651, 653-657, 659-665, 667-671, 673-678, 681-689, 691-693, 695-713, 715-717, 719, 721-727, 729-733, 736-738, 740, 742, 744, 746, 748-752, 755-756, 758-760, 762-769, 771, 774, 776-781, 783-788, 790-796, 798-799, 802, 804-808, 810-815, 817-831, 833-848, 850-855, 857-869, 871-880, 882-900, 903-907, 909, 911-915, 918-920, 922-925, 927, 929, 931-938, 940, 943-945, 947, and 950-953 is increased after infection of wild-type Arabidopsis with Pseudomonas syringae.

To identify pathogen-induced genes whose expression is affected by the mutations, genes for which the ratio of infected mutant/infected wild-type was <0.5 or >2 for at least one mutant were selected from the list of 745 pathogen-inducible probe sets. The limits of 5 and 2 were chosen because changes of at least 2-fold are likely to be significant for impact on disease resistance, and because the false positive rate for the GeneChip™ at 2-fold is 0.2%. This selection yielded a list of 530 probe sets corresponding to genes, the expression of which is induced by Pseudomonas infection in wild-type plants and perturbed in at least one mutant plant (see Tables 4a and 4b below). Thus, the expression of genes comprising SEQ ID NOs:2, 4-6, 11-13, 18, 22-23, 28, 31, 36, 39-43, 45, 47, 49-50, 52, 54-55, 57-58, 60-61, 63-66, 71-72, 74, 77, 81, 83, 85, 87-89, 92, 97, 100-107, 111-112, 114-115, 117-120, 122, 125, 127-128, 134, 138-140, 143-144, 148-151, 153, 156-157, 160, 165, 168-170, 173-174, 176-180, 183, 187-188, 191, 193-194, 197-198, 200, 203-210, 214, 219-224, 227, 230-232, 235-237, 239-240, 243-246, 248-249, 251-254, 256-258, 261, 264, 266-268, 270, 272-275, 277-278, 280, 283, 286-287, 290-293, 297, 302, 305-306, 308-310, 312-316, 321-326, 328-331, 333, 336-339, 341, 345, 351, 353, 355-358, 361-363, 365-366, 368-371, 373, 375, 377, 379-381, 384-385, 388-390, 392, 394-400, 402-406, 410, 412, 415-416, 419-420, 422-425, 429-433, 435-439, 441-443, 445-452, 454, 459-460, 463, 466, 468-470, 473, 481-482, 485-486, 489, 491-494, 497-498, 500-501, 503, 505-506, 508, 510, 513-515, 517, 520-521, 523, 528-529, 531, 533-538, 540, 545-548, 550-551, 553-554, 556-558, 560, 566-567, 575, 580, 582-584, 588-593, 596, 598-600, 602-603, 605-606, 608-610, 612, 614, 616, 620-622, 627-629, 633-634, 636-639, 644, 646, 648-651, 654-657, 659, 661-663, 667, 669, 673-674, 677, 682, 684-687, 689, 691-693, 697, 699, 701, 703-708, 713, 717, 719, 721-727, 730-733, 736, 740, 744, 746, 749-752, 755-756, 758-760, 762-764, 766-769, 774, 776-778, 780-781, 786, 788, 791-796, 799, 802, 804-808, 810-812, 815, 818-821, 823-825, 827-829, 831, 833-836, 838-843, 845, 847-848, 852-853, 855, 858, 860-869, 871-874, 876, 878-880, 884-887, 889, 892-894, 896-900, 904-907, 911-915, 918-920, 922-924, 931, 933, 938, 943-945, 947, and 950-952 is increased after infection of wild-type Arabidopsis, and altered after infection of at least one mutant Arabidopsis, with Pseudomonas syringae.

2. Data Interpretation

Genes that encode regulatory proteins such as transcription factors, protein kinases, calcium binding proteins and the like, are likely to play important roles in disease resistance, as they are likely to affect the expression of multiple defense effector genes. The list of 530 probe sets include 81 that correspond to genes encoding regulatory factors. These are likely to be useful for engineering plants to respond more quickly to pathogen attack by activating expression of defense responses (see Table 5 below). Thus, the expression of genes comprising SEQ ID NOs:39, 52, 60, 63, 81, 83, 106, 107, 115, 117, 118, 168, 174, 176, 179, 204, 207, 208, 220, 221, 248, 258, 268, 275, 280, 309, 323, 326, 329, 351, 419, 422, 429, 430, 432, 459, 460, 468, 469, 473, 500, 505, 506, 508, 529, 531, 533, 535, 538, 545, 553, 602, 606, 608, 610, 614, 616, 634, 654, 655, 684, 686, 687, 691, 717, 751, 752, 766, 777, 815, 831, 834, 835, 839, 841, 847, 876, 884, 906, 920, and 924 is increased after infection of wild-type Arabidopsis, and altered after infection of at least one mutant Arabidopsis, with Pseudomonas syringae.

The mutations nahG, pad4-1, eds5-1, eds4, pad2-1, npr1-1, npr1-3, pad1, FN1-3, eds3, eds8, sid2, eds5-3, FN1-3 and FN3-2 cause enhanced susceptibility to Pseudomonas syringae. Consequently, pathogen-inducible genes whose expression is reduced by one of these mutations are likely to be important for resistance to Pseudomonas syringae and possibly other bacterial pathogens. These 333 probe sets are shown in Table 6 (below). Therefore, the expression of genes comprising SEQ ID NOs:12-13, 18, 23, 36, 39-40, 43, 45, 50, 52, 57-58, 60-61, 64, 71-72, 81, 87-89, 97, 100, 102-105, 107, 111-112, 115, 119-120, 122, 125, 127-128, 140, 144, 148-150, 153, 165, 168-169, 176-177, 179, 183, 188, 191, 193-194, 197-198, 203-206, 208-209, 214, 219-222, 227, 230, 232, 237, 244-246, 248-249, 251-253, 258, 261, 264, 266, 268, 273-275, 283, 287, 290, 293, 297, 302, 305-306, 308, 312-315, 321-322, 324, 326, 330, 333, 338, 341, 345, 353, 356-358, 362-363, 366, 369, 371, 375, 377, 380, 384-385, 389, 392, 394-395, 398-399, 402-404, 406, 410, 415, 419, 422, 425, 429-430, 433, 435-439, 443, 445-452, 454, 463, 466, 468-470, 473, 486, 489, 491-492, 494, 498, 500-501, 503, 508, 513-514, 517, 529, 533-538, 548, 550, 553-554, 4556-558, 566, 575, 580, 582-583, 590-591, 593, 600, 602, 609-610, 612, 614, 620-622, 627-629, 637-638, 644, 649, 654-657, 659, 663, 667, 669, 673-674, 677, 684-685, 689, 691-693, 699, 703-705, 708, 719, 721, 724-726, 730-732, 744, 746, 749-750, 752, 755-756, 758, 760, 762-764, 767, 769, 774, 780-781, 786, 788, 791-792, 794-796, 799, 804-808, 810-812, 815, 818-819, 823, 828-829, 833, 840-841, 843, 847, 852-853, 858, 860, 862-865, 867-868, 872-874, 876, 885-887, 889, 892-894, 896-900, 904-905, 907, 911-914, 918-920, 922-924, 931, 933, 938, 947, 950, and 952 is increased after infection of wild-type Arabidopsis, and altered after infection of at least one mutant Arabidopsis having a mutation that results in enhanced susceptibility to Pseudomonas (nahG, pad 4-1, eds 5-1, eds4, pad2-1, np4 1-1, npr 1-3, pad1, FN1-3, eds3, eds8, sid2, eds5-3, NF1-3 and FN3-2).

The mutations coi1 and ein2 block jasmonate and ethylene signaling, respectively. Jasmonate and ethylene-dependent disease resistance responses are known to be important for resistance to the fungal pathogens Alternaria brassicicola and Botrytis cinerea, and may also be important for resistance to other necrotrophic fungal pathogens. Alternaria and Botrytis are distantly related, yet plant resistance to these fungi is controlled similarly, suggesting that jasmonate- and ethylene-dependent responses function to limit growth of a wide range of fungal pathogens. Consequently, pathogen-induced genes whose expression is reduced in coi1 and ein2 mutants are likely to be important for resistance to these necrotrophic fungal pathogens. These 296 probe sets are shown in Table 7 (see below). Hence, the expression of genes comprising SEQ ID NOs:2, 4, 6, 11-13, 18, 22-23, 31, 41-43, 49-50, 54, 57-58, 61, 64-66, 71-72, 74, 77, 85, 87, 89, 92, 97, 101, 103, 106-107, 112, 114, 117-119, 125, 128, 134, 138, 143, 149, 151, 156-157, 165, 169-170, 174, 176-180, 187-188, 191, 193, 206, 208, 219-220, 222, 224, 231, 236, 239, 243-245, 251-254, 256-257, 267, 272, 287, 290, 292, 297, 302, 312-313, 315-316, 321-322, 324-325, 328, 330, 345, 351, 353, 355-357, 362-363, 366, 368-371, 373, 375, 379, 381, 384, 388-390, 392, 395-400, 405, 410, 415-416, 419, 422, 424, 431-432, 435-436, 438-439, 447, 459-460, 470, 473, 481-482, 489, 491, 493-494, 500-501, 505-506, 513-514, 517, 520-521, 523, 528-529, 531, 535, 537-538, 540, 545-548, 551, 553-554, 557-558, 566, 575, 580, 582, 584, 589, 591, 593, 596, 598-599, 603, 605, 608-609, 612, 628, 633-634, 636-637, 639, 646, 648, 650-651, 656, 661, 663, 667, 674, 685-687, 689, 691, 693, 697, 699, 701, 705, 707, 713, 723-724, 726, 736, 740, 749, 751-752, 756, 758-759, 764, 766-768, 774, 776, 778, 780, 792-796, 799, 802, 806, 810-812, 818, 820-821, 825, 827-829, 833-836, 838-839, 841-843, 848, 855, 860-861, 866, 868-869, 871, 873-874, 876, 878-880, 889, 892, 898-900, 904-905, 907, 915, 918, 922, 924, 933, 943-945, 947, and 951 is increased after infection of wild-type Arabidopsis, and altered after infection of at least one mutant Arabidopsis having a mutation in a gene whose expression is important for resistance to necrotrophic fungi (a mutation that blocks or interferes with jasmonate and ethylene signaling such as col1 and ein2). Accordingly, these genes are useful to improve the resistance of plants to fungal infection.

The mutations nahG, pad4-1, sid2, eds5-1, eds5-3, and eds4 are known to interfere with salicylic acid dependent signaling. Such signaling is known to be important for resistance to the bacterial pathogen Pseudomonas syringae, the oomycete pathogen Peronospora parasitica, the viral pathogen tobacco mosaic virus, as well as various other plant pathogens. Consequently, pathogen-induced genes whose expression is reduced by one of the mutations that block salicylate signaling are likely to be important for disease resistance, and useful for engineering improved disease resistance. These 288 probesets are shown in Table 8 (see below). Therefore, the expression of genes comprising SEQ ID NOs: 12-13, 18, 23, 36, 39-40, 43, 45, 50, 52, 57-58, 60-61, 64, 71-72, 81, 87-88, 100, 102-105, 107, 111-112, 115, 119-120, 122, 125, 127-128, 140, 148-150, 153, 168-169, 176-177, 188, 191, 193-194, 197-198, 203-206, 209, 219-222, 227, 232, 237, 244-246, 248-249, 251-253, 258, 261, 264, 266, 268, 273-275, 283, 287, 290, 293, 297, 302, 305-306, 308, 312-315, 324, 326, 330, 333, 341, 345, 353, 356, 358, 366, 371, 375, 377, 380, 385, 389, 392, 394, 398, 402-404, 406, 410, 415, 419, 425, 429-430, 433, 435-438, 443, 445-447, 449-452, 454, 463, 466, 468-470, 473, 486, 489, 492, 494, 498, 500-501, 503, 508, 513-514, 517, 533-538, 548, 550, 553-554, 57-558, 566, 575, 582-583, 590-591, 593, 600, 602, 609-610, 612, 620-622, 627-629, 637-638, 644, 649, 654-657, 659, 667, 669, 673, 677, 684, 689, 692-693, 703-705, 719, 721, 724-726, 730-732, 744, 746, 749-750, 752, 755-756, 760, 762-764, 767, 769, 774, 780-781, 786, 788, 791-792, 795-796, 805-808, 810-812, 815, 818-819, 823, 828, 833, 840-841, 843, 852-853, 858, 860, 862-865, 867-868, 872-874, 876, 887, 889, 893-894, 896-898, 900, 905, 907, 911-914, 918-920, 922-923, 931, 933, 938, 947, 950, and 952 which is increased after infection of wild-type Arabidopsis and altered after infection of at least one mutant Arabidopsis having a mutation in a gene that interferes with salicylic acid dependent signaling (nahG, pad4-, sid2, eds5-1, eds5-3 and eds4). Thus, these genes are particularly useful to improve the resistance of plants to infection by more than one pathogen including bacteria, oomycetes and viruses, such as TMV.

### EXAMPLE 3

### Further Analysis of the Pathogen Response and Comparison of the Response in Different Ecotypes

Materials and Methods

Arabidopsis ecotypes (or accessions) (the wild-types of all the Arabidopsis ecotypes used here have wild-type alleles of RPS2 and RPM1).

Col, Columbia-0

Ler, Landsberg erecta

Ws, Wassilewskija

Arabidopsis Mutants and Transgenics

Col rps2-101C, a loss-of-function mutant of the resistance gene RPS2 in Col background.
NahG, transgene for salicylic acid hydroxylase (inactivating salicylic acid). Col background.
ndr1-1, null mutant allele of NDR1 (non-race specific disease resistance). The mutation strongly affects RPS2-mediated resistance and partially affects RPM1-mediated resistance. Col background.
   Bacterial Strains
Pst, Pseudomonas syringae pv. tomato DC3000 (virulent strain of Arabidopsis)
Psm, P. syringae pv. maculicola ES4326 (another virulent strain of Arabidopsis)
Psp, P. syringae pv. phaseolicola NPS3121 (very weak pathogen of Arabidopsis)
   Avirulence (avr) Genes of P. syringae

avrRpt2: corresponding to the Arabidopsis resistance (R) gene RPS2

avrB: corresponding to the Arabidopsis resistance (R) gene RPM1

Experimental Protocols

A. Gene for Gene Resistance (6 Hours After Treatment)

| plant | treatment |
|---|---|
| Col WT | H₂O |
| Col WT | Pst |
| Col WT | Pst/avrRpt2 |
| Col rps2-101C | H₂O |
| Col rps2-101C | Pst |
| Col rps2-101C | Pst/avrRpt2 |
| Ws WT | H₂O |
| Ws WT | Pst |
| Ws WT | Pst/avrRpt2 |

B. Differences in the Response to Bacterial Pathogens Among Ecotypes (3, 6, and 9 Hours After Treatment)

| Plant | treatment |
|---|---|
| Col | H₂O |
| Col | Pst |
| Col | Pst/avrRpt2 |
| Ler | H₂O |
| Ler | Pst |
| Ler | Pst/avrRpt2 |
| Ws | H₂O |
| Ws | Pst |
| Ws | Pst/avrRpt2 |

Note that overall results for Cvi were very similar to Ler.

C. Genetic Factors that Affect the Plant Response to Incompatible Interactions (3, 6, and 9 Hours After Treatment)

| plant | treatment |
|---|---|
| Col | H₂O |
| Col | Pst |
| Col | Pst/avrRpt2 |
| Col | Pst/avrB |
| Col | Psm |
| Col | Psm/avrRpt2 |
| Col | Psp (not 9 hours) |
| Col | Psp/avrRpt2 (not 9 hours) |
| Col NahG | Pst |
| Col NahG | Pst/avrRpt2 |
| Col NahG | Pst/avrB |
| Col ndr1-1 | Pst |
| Col ndr1-1 | Pst/avrRpt2 |
| Col ndr1-1 | Pst/avrB |

Results

Four hundred sixty-five genes were specifically/preferentially induced in the incompatible interaction (WT and Pst/avrRpt2), and 616 genes were specifically/preferentially repressed in the incompatible interaction. Examples of these genes are provided in Tables 10 and 13. Gene expression patterns in the incompatible interaction in Col and Ws were significantly different, indicating that the genetic diversity among ecotypes can affect gene regulation during the incompatible interaction significantly. In comparison, a relatively small number of genes (314 genes for induction, 167 genes for repression) were affected at this time point during the compatible interactions (but not preferential to the incompatible interactions). A comparison of the results in three genetically different compatible interactions (WT and Pst, rps2 and Pst, rps2 and avrRpt2) revealed that 25 genes were repressed in an avrRpt2-dependent manner (see Table 9). Thus, the expression of genes comprising SEQ ID NOs:1, 15, 19, 20, 24, 26, 27, 34, 38, 51, 56, 59, 67-69, 99, 116, 155, 159, 182, 212, 284, 372, 444, and 789 is down-regulated (repressed) in an avrRpt-2-dependent manner in Arabidopsis. These genes are good candidates to be involved in avrRpt2 virulence functions (in rps2 plants).

Genes that were induced in rps2 plants after infection irrespective of avrRpt2 indicate a function of RPS2 other than an interaction with avrRpt2. Thus, global gene expression profiling can identify large and minor trends in gene regulation and is useful in gene discovery.

One general phenomenon when plants are resistant to a pathogen is the early response of pathogen-responsive (induced or repressed) genes compared to plants that are susceptible to infection. This has been proposed based on observing expression of a very limited number of genes, but it has not been proven as a global trend. To examine the results from early incompatible interactions and late compatible interactions, 4 week old Col-0 plants with well expanded leaves were infected with a high dose (OD₆₀₀=0.02) or low dose (OD₆₀₀=0.002) of P. syringae and samples collected at 6 or 30 hours, respectively. The two expression patterns were similar. The correlation values between the late compatible and incompatible interaction at either 6 hours, 9 hours or the average of 3-9 hour time points was 0.71, 0.72 and 0.75, respectively.

The majority of genes that did not respond within 9 hours after infection of a virulent strain but that responded in 30 hours (Pst or Psm, for Pseudomonas syringae pv. tomato DC3000 and Pseudomonas syringae pv. maculicola ES4326, respectively; the plant is susceptible to these strains) responded within 6 hours after infection of an avirulent strain (Pst/avrRpt2; Pst carrying the avirulence gene avrRpt2; the plant is resistant to this strain). This strongly suggests that early response of the pathogen-responsive genes is crucial for the plant to be resistant.

A comparison of the differences in the expression patterns of the 2 primary ecotypes of Arabidopsis' response to infection provides a further way to identify which genes have a more universal role (unchanged expression pattern) and which may be very specific to a particular plant ecotype involved in a very specific gene-for-gene interaction. For example, responses that are common between two ecotypes may be important for resistance. Genes that show the same pattern in both ecotypes may be part of more universal, or commonly-used, mechanisms involved in plant-pathogen interactions. Responses that are different may indicate that the two ecotypes use different combinations of responses to achieve resistance. This implies that a variety of genes can participate in plant-pathogen interactions. Nevertheless, ecotype-specific responses are expected to have counterparts in other plant species.

The differences in resistance response between ecotypes can be used for improving resistance in plants. In responses that are different between ecotypes, using the methods and compounds of the invention, such a response can be added to (induced or repressed) the response seen in the ecotype which does not normally use that response. This will likely give the plant a more robust or a wider range of resistance.

Table shows a comparison of gene expression in 4 ecotypes, i.e., Col-0, Ws-2, Cvi and Ler in response to infection. Table 10A shows the expression data for 9 probe sets corresponding to genes that are specifically induced at 3 hours after incompatible infection of four different ecotypes of Arabidopsis with P. syringae pv. tomato DC3000. Table 10B shows expression data for 18 probe sets corresponding to genes that are induced by 6 hours but not at 3 hours after incompatible infection of four different ecotypes of Arabidopsis with three different bacterial strains, i.e., P. syringae pv. tomato DC3000. Table 10C illustrates the expression data for 6 probe sets corresponding to genes that are activated by P. syringae at 6 hours post-infection. Most of the genes are compatible interaction-specific or -preferential.

Four week old plants with fully expanded leaves were infected and samples collected at 3 or 6 hours post-infection (OD₆₀₀=0.02). Some common patterns were observed. At 3 hours after infection of an avirulent strain, Pst/avrRpt2, the overall qualitative gene expression patterns were very similar for all the ecotypes tested. Common responses to Pst/avrRpt2 could be important for gene-for-gene resistance and so may be useful to identify targets for reverse genetics. Quantitative and qualitative differences in the response were noted, indicating that there are qualitative and/or quantitative differences in the signal transduction mechanisms that regulate the response among the ecotypes. Such signal transduction mechanism differences are attributed to genetic differences among the ecotypes.

In particular, early inducible genes (3 hours) in the incompatible interaction were identified (70 genes are common in all the ecotypes, and 360 genes if selected for induced in at least one ecotype). One group of the early genes (38 genes in Col) were repressed to the control level by 6 hours. These genes did not respond in the compatible interaction at 3 hours and were repressed below the control level in the compatible interaction by 6 hours. This suggests that shutting down these genes in the incompatible interaction by 6 hours could be caused by defense response inhibiting factor(s) delivered by bacteria. Another group of the early genes were expressed even higher at 6 hours in the incompatible interaction. One hundred eighty-eight genes showed significant induction or repression at 3 hours in the compatible interaction in at least one of the ecotypes. Of these, 3 induced genes and 3 repressed genes were induced or repressed in all three ecotypes.

At 3 hours in the incompatible interaction, a major difference among the ecotypes was quantitative; overall expression patterns were very similar, but overall fold change amplitudes were clearly in the order of Ws>Col>Ler. Thus, in this type of analysis it is not appropriate to analyze datasets by comparing the genes from different datasets that are selected by a certain cut-off value (e.g., 2.5-fold difference). This fold change difference was mainly caused by differences in the basal expression of these genes. In fact, a strong negative correlation in each gene was found between the relative basal expression level in Ws (relative to the other ecotypes; Pearson correlation −0.78) and response in the incompatible interaction (especially at 3 hours) and a moderate positive correlation between the relative basal expression level in Ler and response in the incompatible interaction (Person correlation 0.38) (almost no correlation for the relative basal expression level in Col; Person correlation 0.10). These observations indicate that Ws has the tightest regulation of these incompatible interaction-responsive genes, and Ler has the loosest. Another interesting observation is that the relative susceptibility to a virulent strain (Pst) was in the order of Ws>Col>Ler. Although it is unknown whether these two phenomena are controlled by same gene(s), it is conceivable that leaky expression of early response genes (in Ler) confers relative resistance to a virulent strain. At 6 hours in the incompatible interaction, the gene expression pattern for Col was significantly different from the other ecotypes.

Moreover, different ecotypes may use a different but overlapping set of responses to achieve resistance against the same pathogen. Gene expression profiling can thus reveal ecotype differences. Therefore, it is possible to isolate the genes responsible for these differences in regulatory mechanisms using ecotype differences in gene expression as a phenotype, by a map-based cloning approach.

For example, a majority of the incompatible response-inducible genes have lower basal levels in ecotype Ws and higher basal levels in ecotype Ler. Among the numerous genes, a few genes that display large differences in the basal level in two ecotypes are chosen. The large differences in expression level constitute easy-to-score phenotypic markers. Ws and Ler are crossed to obtain F2 populations. The larger the F2 population is, the better resolution in the map position can be obtained. For each of the F2 plants, expression levels of the chosen phenotypic marker genes are measured and physical markers that distinguish these ecotype genomes are scored. The map position of the responsible gene is determined by analyzing the linkage between the phenotype and the physical markers. If more than a single gene is responsible for the ecotype difference and each of the genes has a quantitative effect on the phenotype, quantitative trait locus (QTL) analysis can be used for mapping. Instead of using F2 populations, the use of recombinant inbred lines (RILs) between the ecotypes of interest may facilitate the analysis, especially using RILs that are already mapped for recombination points. Once the gene(s) responsible for the phenotype is mapped, a combination of increasing the map resolution, sequencing the chromosomal region identified by mapping in both ecotypes, and gene transfer from one ecotype to the other leads to isolation of the gene.

If the phenotype of interest in gene expression depends on bacterial infection, such as expression of ecotype Col-specific inducible genes at 6 hours after infection of Pst/avrRpt2, expression of the corresponding phenotypic marker genes (e.g., genes that show good difference in induction between Col and Ler) can be measured at an appropriate time after bacterial infection.

Differences in gene expression patterns between two virulent strain backgrounds (Pst and Psm) are relatively small. Gene expression patterns for Pst/avrRpt2 and Pst/avrB were quite similar at 3 hours, but the difference increased at 6 hours. Psp (no avr) shows similar expression pattern to incompatible bacteria although the amplitude of fold difference was smaller in general. This suggests that Psp, which does not induce the HR in the plant, is still recognized by the plant and induce major part of the defense response seen during the incompatible interaction. It also suggests that plants monitor the effect of the defense response and that if it seems effective (bacteria do not grow like Psp), the plant does not go for a full-blown defense response.

Preferred Genes

Preferred early inducible genes were selected as induced >2.5 fold (except for 2 fold for Psp at 6 hours) in all of the following datasets: Pst/avrRpt2 at 3 hours in Col, Ws, and Ler; Pst/avrRpt2 at 3 hours, Psm/avrRpt2 at 3 hours, Psp at 6 hours, and Pst/avrB at 3 hours, relative to the water control, as well as estradiol-inducible (avrRpm1 at 0, 45, and 120 minutes and avrRpt2 at 0, 45, and 120 minutes, where the fold change was relative to the appropriate resistance gene mutant carrying the same transgenes. Among these genes, the genes were ranked according to genes that are not induced by SA or BTH and not induced in late time points with Psm. Regulatory genes were given higher rankings (see Table 11). Hence, the expression of genes comprising SEQ ID NOs:17, 70, 76, 81, 84, 109, 123, 144, 160, 230, 265, 268, 269, 271, 323, 333, 385, 427, 428, 430, 457, 505, 569, 597, 602, 606, 616, 708, 730, 741, 812, 862, and 942 is induced early after infection of different Arabidopsis ecotypes with Pseudomonas syringae pv tomato DC3000, P. maculicola ES4326 and P. phaseolica NPS3121 (at 3 or 6 hours) or is estradiol inducible (at 45 or 120 minutes).

Preferred early repressible genes were selected as repressed >2.5 fold (except for >2 fold for Psp at 6 hours) in all of the following datasets: Pst/avrRpt2 at 3 hours, Psm/avrRpt2 at 3 hours, Psp at 6 hours, and Pst/avrB at 3 hours) and Pst/avrRpt2 at 3 hours in Col (the fold change was relative to the appropriate water controls). Among them, the genes were ranked in order of expression (highest to lower levels of expression) (see Table 12). Thus, the expression for genes comprising SEQ ID NOs:30, 73, 282, 541, 640, 679, 761, 870, 917, and 930 is repressed early after infection of Arabidopsis with Pseudomonas syringae pv tomato DC3000, P. maculicola ES4326 and P. phaseolica NPS3121.

Other genes are induced/repressed during incompatible interactions at 3 and/or 6 hours after inoculation of bacteria. Preferred genes in this group were selected as induced/repressed >2.5 fold in the incompatible interaction compared to water inoculated control and 2> fold compared to the corresponding compatible interaction at 3 and/or 6 hours after inoculation with Pst/avrRpt2 and Pst/avrB, and Psm/avrRpt2 and Pst/avrRpt2, in all four ecotypes (see Tables 13a and 13b). Hence, the expression of genes comprising SEQ ID NOs:21, 44, 46, 60, 86, 91, 93, 106, 110, 119, 122, 130, 131, 161, 166, 167, 168, 171, 176, 200, 203, 213, 225, 227, 248, 261, 262, 266, 274, 285, 300, 301, 302, 320, 326, 341, 345, 348, 349, 360, 366, 378, 615, 618, 406, 409, 422, 425, 441, 443, 446, 449, 454, 461, 475, 476, 485, 500, 511, 512, 527, 533, 543, 545, 549, 550, 552, 567, 575, 590, 608, 611, 625, 643, 656, 659, 666, 668, 671, 680, 690, 704, 706, 711, 721, 728, 738, 757, 791, 807, 811, 813, 827, 857, 864, 868, 875, 881, 893, 901, 905, 908, 912, 939, 941, 951, and 952 is induced in an incompatible interaction at 3 and/or 6 hours after infection of four Arabidopsis ecotypes with Pseudomonas syringae pv tomato DC3000, P. maculicola ES4326 and P. phaseolica NPS3121, while the expression of genes comprising SEQ ID NOs:7, 33, 82, 136, 141, 154, 185, 189, 199, 202, 434, 471, 483, 499, 516, 530, 578, 586, 631, 658, 694, 714, 718, 734, 770, 772, 816, and 916 is decreased in an incompatible interaction at 3 and/or 6 hours after infection of four Arabidopsis ecotypes with Pseudomonas syringae pv tomato DC3000, P. maculicola ES4326 and P. phaseolica NPS3121.

Garlic T-DNA insertion lines corresponding to these genes are searched by BLAST. Global expression profiling after infection with one of two different pathogens (P. syringae and Alternaria brassicicola) may be employed as a phenotyping method. Transgenic plants for overexpression, underexpression, and conditional overexpression of selected genes are also prepared.

### EXAMPLE 4

### Promoters of Genes Responsive to Pathogen Infection

In many cases the major outcomes of plant-pathogen interactions are largely determined by how plants react in an early stage. Therefore, it is useful to isolate promoters that rapidly react to pathogen attack for use in expressing proteins that provide tolerance or resistance to pathogen attack.

Genes were selected according to the conditions described below based on the results of a GeneChip™ analysis. These genes were particularly selected for a high level of induction in the avrRpt2-RPS2 interaction and for a very low mRNA level in the absence of pathogen attack among four Arabidopsis ecotypes tested (Col, Ws, Ler, and Cvi). The genes were also analyzed to determine if their expression was similar in other combinations of incompatible interactions (three different bacterial strain backgrounds: P. syringae pv. tomato DC3000, P. syringae pv. maculicola ES4326, and P. syringae pv. phaseolicola NP3121; three different avirulence genes: avrRpt2, avrB, and avrRpm1; and direct expression of avirulence genes in plants using an estradiol-inducible system). For each gene, the 1.2-kb sequence upstream of the initiation codon is provided in SEQ ID NOs: 1047-1095.

Preferred Highly Inducible Promoters

Promoters were selected that had low basal expression level (i.e., uninduced level) in all the ecotypes (Col, Ler, Ws, and Cvi) and high inducibility in Col. Five such promoters of genes represented by the probe sets in Table 14 were identified: the promoters of germin precursor-like oxalate oxidase gene, extra-large G protein gene, PR-1, EREBP5 gene, and a C2H2-type zinc finger protein gene were chosen. The promoters for the germin-precursor like oxalate oxidase gene and PR-1 gene are relatively slow response promoters (no induction 3 hours after infection), but have high induction by 6 hours. The extralarge G protein gene is an intermediate in terms of response time, but maintains high expression over time. The other two are useful as early transient response promoters (good induction by 3 hours, but shut down by 6 hours) in the incompatible interaction (wild type plant infected with Pst/avrRpt2). Promoter sequences comprising SEQ ID NOs:1046-1095 and 1047-1055 correspond to genes comprising one of SEQ ID Nos: 17, 21, 80, 81, 109, 156, 174, 176, 221, 227, 296, 302, 303, 306, 333, 340, 360, 500, 505, 524, 575, 601, 602, 614, 628, 687, 733, 782, 811, 835, 862, 900, 905, 912, and 109, 306, 524, 600, 875, 912, 913, 941 and 942, respectively. Promoter-LUC reporter fusions are prepared and tested in a transient expression system using biolistic co-bombardment of avrRpt2 gene.

Promoters Responsive to Particular Pathogens

Proteins that are useful for protecting plants from pathogen attack may have deleterious effects on plant growth if expressed constitutively. Consequently, it is desirable to have promoter sequences that control gene expression in such a way that expression is absent or very low in the absence of pathogens, and high in the presence of pathogens.

Wild-type Arabidopsis plants (ecotype Columbia) were either mock-infected or infected with the bacterial pathogen Pseudomonas syringae pv. maculiola strain ES4326 (2×10⁴ cfu per square centimeter of leaf). After 30 hours, samples were collected, and RNA was purified. This procedure was repeated three times independently, and the RNAs from corresponding samples were pooled, in order to reduce the impact of variation due to uncontrolled variables. The two pools of RNA representing mock-infected and infected plants were then used for gene expression profiling using an Arabidopsis GeneChip®. This entire procedure was repeated three times, yielding three sets of GeneChip® data representing a total of nine independent experiments.

To identify promoter sequences that are likely to be useful for driving expression of transgenes in plants in response to pathogen attack, genes were selected whose expression level was less than 40 in all of the mock-infected samples and whose expression level was greater than 400 in all of the infected samples. The value of 40 was chosen arbitrarily as a low expression level and the value of 400 was chosen arbitrarily as a reasonably high expression level. Thirty-seven genes met these criteria and promoter sequences could be identified for 36 of them. Table 15 indicates the identifying probe set number for these 36 genes, the corresponding Arabidopsis gene, the mean expression level of each gene in mock-infected plants, the mean expression level of each gene in infected plants, and the fold induction in expression of each gene after infection. For 11 genes, expression in mock-infected plants was undetectable, so it was not possible to calculate fold induction. Therefore, the expression of genes comprising SEQ ID NOs:104-106, 119, 123, 129, 131, 151-152, 183, 191, 198, 200, 227, 249, 274, 302, 358, 415, 481, 547, 566, 582, 628, 633, 639, 656, 673, 793, 818, 827, 864, 874, 880, and 904-905 is induced in Pseudomonas syringae pv. maculiola-infected Arabidopsis.

It is possible that promoters that strongly activate gene expression in response to infection by a bacterial pathogen might be different from promoters that strongly activate gene expression in response to infection by a fungal pathogen. To test this possibility, a second GeneChip® experiment was conducted, in which wild-type Arabidopsis plants (ecotype Columbia) were mock-infected or infected with the fungus Botrytis cinerea. Samples were collected at 0, 12, 36, 60, and 84 hours after infection, RNA was purified and used for expression profiling using an Arabidopsis GeneChip®. To identify useful promoters, genes were selected whose expression level was less than 40 in mock-infected samples from all time points and whose expression level was greater than 400 in infected plants at 84 hours after infection. Twenty-three genes met these criteria, and promoter sequences could be identified for 21 of them. These genes are described in Table 16, with their identifying probe set number, the corresponding Arabidopsis gene, the mean expression level of each gene in mock-infected plants, and the expression level of each gene in infected plants at various times after infection. Among these 23 genes, 11 genes were previously identified in the search for genes whose expression was strongly induced by Pseudomonas syringae infection. These 11 genes correspond to identifying codes 12989, 13015, 13100, 13215, 13565, 14609, 16649, 16914, 19284, 19991, and 20356. Hence, the expression of genes comprising SEQ ID NOs:18, 71, 119, 123, 129, 151, 191, 244, 245, 302, 545, 547, 562, 566, 637, 653, 747, 756, 774, 793, 842, 864, and 905 is induced in Botrytis cinerea-infected Arabidopsis.

The promoter sequences for the 25 genes that were only identified in the P. syringae data set are shown in SEQ ID NOs:1001-1025. The promoter sequences for the 10 genes that were only identified in the B. cinerea data set are listed in SEQ ID NOs:1026-1035) The promoter sequences of the 11 genes that were identified in both data sets are listed in SEQ ID NOs:1036-1046. The 11 promoter sequences that were identified in both data sets are most likely to be useful for driving expression of transgenes in response to attacks by various pathogens, as these promoters are activated in response to attack by either Pseudomonas syringae or Botrytis cinerea, two very different pathogens. The other promoters may also be useful for driving expression of transgenes that are efficiently expressed in response to infection by certain types of pathogens.

Further, orthologs of the Arabidopsis promoters are also useful to drive expression of transgenes. To identify the orthologous promoter, a BLAST search for orthologous genes was conducted. To identify the ortholog, the alignments from the BLAST search are used to determine the range of nucleotides showing homology to the Arabidopsis gene. The coding sequences shown at the beginning of each search result that contain regions corresponding to the nucleotides showing homology are likely orthologous genes. Orthologous promoter sequences may be isolated by any method known to the art, e.g., cloning of genomic DNA 5′ to the ATG in orthologous genes identified in a computer assisted database search or hybridization of a probe comprising any one of SEQ ID NOs: 1001-1046 to genomic plant DNA.

### EXAMPLE 6

### Genes the Expression of which are Altered by Viral Infection

To identify host genes that are commonly up or down regulated during local RNA or DNA virus infection, gene expression profiling was employed. The host genes may include host factors that are induced by viral infection, e.g., activated host defense genes, suppressed by viral infection, e.g., suppressed host defense genes, genes involved in symptom development, as well as genes regulated by virus inducible promoters. Once the genes are identified, the function of each is then determined. Reverse genetics is then employed to examine the effect of mutations on these genes during virus infection.

Experimental Procedure

Arabidopsis thaliana (Columbia-0 (Col-0) were grown in a Conviron growth chamber to 4 weeks of age. The growth conditions were 22° C., 12 hour day length and 75% relative humidity. At least four rosette leaves of twenty plants were inoculated with one of five viruses or a mock control (120 plants total). The viruses were turnip vein clearing virus (TVCV), a tobamovirus, an oil seed rape mosaic virus (ORMV), a tobamovirus, tobacco rattle tobravirus (TRV), a tobravirus, cucumber mosaic virus strain Y (CMV-Y), a cucumovirus, and turnip mosaic virus (TuMV), a potyvirus. Each virus was diluted to approximately 0.5 to 1.0 μg/ml in 10 mM potassium phosphate buffer pH 7.2 (or 20 mM Tris-HCl pH 8.0 for the TuMV). The phosphate buffer was used as the mock infection control for the experiments. Inoculated Col-0 leaves were first dusted with carborundum then 10 μl of virus solution or phosphate buffer were pipetted onto the leaf surface. The virus solution or phosphate buffer alone were then rubbed into the leaf surface using a gloved finger, and the leaf surfaces were washed with distilled water at about 10 minutes post inoculation.

Inoculated leaf tissue was removed from each plant at 1, 2, 4 and 5 days post inoculation (dpi), weighed, snap frozen in liquid nitrogen and stored at −80° C. Total RNA was extracted from leaf tissue by the RNAwiz method (Ambion, Inc.) and further purified using the RNeasy method (Qiagen, Inc.). RNA was diluted to 1 μg/ml and labeled as a probe for Affymetrix GeneChip hybridization according to Affymetrix protocol for synthesizing labeled copy RNA (cRNA) (see Example 1). Labeled cRNA for each virus or mock treatment was hybridized to an Affymetrix GeneChip containing sequences corresponding to 8775 Arabidopsis genes. The hybridization data was then analyzed using Affymetrix GeneChip software.

Arabidopsis genes that were induced by at least 2-fold in all virus treatments were identified by importing the data into Microsoft Excel and then subjecting the data to selection criteria. Within each time point, the expression level of a gene exceeded 25 and the fold change was greater than 2 by comparison with the mock-infected treatment. Thus, for genes that were induced by all five viruses, the expression level exceeded 25 and the fold change was greater than 2 for all five viruses. For genes that were repressed by at least 2-fold, the expression level of the gene must exceed 25 in the mock-infected treatment and the fold change must be less than 2 in all of the five virus treatments.

Results

A gene chip from Affymetrix having oligonucleotides corresponding to approximately 8,100 Arabidopsis genes was used with labeled cRNA obtained from plant cells infected with a selected viruses at different days post-infection (dpi). For example, for Arabidopsis, the RNA may be obtained from Arabidopsis infected with potyvirus, tobamovirus, tobravirus, cucumovirus or geminivirus. After hybridization, laser scanning is employed to detect expression levels and the data obtained is then analyzed. For genes that are induced in response to viral infection, genes that are expressed at levels greater than, for example, 2 fold over control, are selected. Alternatively, for genes that are suppressed in response to viral infection, genes that are expressed at levels lower than control are selected. The advantages of a gene chip in such an analysis include a global gene expression analysis, quantitative results, a highly reproducible system, and a higher sensitivity than Northern blot analyses. Moreover, a gene chip with Arabidopsis DNA has a further advantage in that the Arabidopsis genome is well characterized.

Data obtained from probe sets which correspond to genes upregulated or downregulated in response to infection by all 5 viruses reveiled forty-six genes that were downregulated and 126 that were upregulated in response to viral infection (Tables 17 and 18). Once the induced and/or suppressed genes are identified, the functions of the genes are then characterized by standard methodology.

Therefore, the expression of genes comprising SEQ ID NOs:14, 48, 53, 98, 217, 226, 295, 327, 343, 352, 369, 404, 407, 418, 453, 458, 465, 472, 480, 488, 495, 507, 509, 513, 514, 559, 561, 581, 604, 607, 613, 641, 652, 672, 720, 735, 739, 743, 745, 754, 773, 803, 832, 849, 948, and 949 is downregulated after viral infection, and the expression of genes comprising SEQ ID NOs:3, 51, 54, 60, 61, 66, 75, 76, 78, 88, 95, 96, 101, 106, 108, 123, 126, 128, 129, 131, 137, 145-147, 150, 158, 169, 170, 172, 173, 197, 200, 216, 219, 224, 230, 233, 237, 249, 250, 263, 274, 275, 276, 299, 307, 323, 333, 342, 346, 359, 382, 383, 387, 391, 393, 401, 411, 415, 427, 442, 455, 459, 466, 477, 481, 485, 487, 502, 511, 515, 525, 534, 539, 542, 560, 571, 577, 579, 584, 587, 595, 600, 627, 638, 645, 654, 659, 668, 681, 688, 695, 696, 706, 708, 730, 742, 753, 775, 785, 786, 792, 797, 800, 801, 809, 817, 819, 820, 823, 827, 847, 856, 875, 885, 896, 902, 910, 921, 922, 923, 925, 926, 928, 946, and 952 is upregulated after viral infection.

The orthologs of these Arabidopsis sequences to other plant genes was determined.

A summary of the probe sets corresponding to genes, the expression of which is altered after infection of Arabidopsis with a pathogen is shown in Table 19.

### EXAMPLE 7

### Identification of Gene Products that are Modulated upon Infection of a Chenopodium Cell with a Virus

Of the many disease resistance mechanisms that can be studied, the HR (hypersensitive resistance) system of Chenopodium spp. is attractive because of the broad-spectrum virus resistance it confers. This is shown by the ability of members of the bromo-, como-, cucumo-, ilar-, alfamo-, nepo-, sobemo-, tombus-, tymo-, carla-, clostero-, hordei-, potex-, poty-, tobra- and tobamovirus groups to elicit local lesion HR on Chenopodium spp. (CMI/AAB Description of Plant Viruses, 1984; Cooper et al., (1995)). In many instances, the HR completely blocks viral spread. However, certain viruses can break through the hypersensitive response and move from one species of Chenopodium to another. The ability of some viruses to infect more than one species of Chenopodium provides an opportunity to isolate genes that provide a cell with resistance to viral infection.

The genetic mechanisms of Chenopodium spp. HR involve a number of factors. These factors can be studied to further understand the hypersensitive response and the mechanism through which the response acts. There are some similarities between the products of Chenopodium spp. genes and gene products involved in common defense signaling pathways in other plants. These similarities allow comparisons to be made between Chenopodium and these other plants. One example includes genes that are induced upon viral infection during HR in C. foetidum (Visedo et al., (1990).

Additionally, some circumstantial experimental evidence suggests that Chenopodium HR may be somewhat similar to tobacco N gene HR (Whitham et al., 1994). Movement defective tobacco mosaic tobamovirus (TMV) replicates within an inoculated cell of a tobacco plant with an N gene, but fails to move from cell to cell (Cooper et al., 1996). Hypersensitivity is not induced, thus replication alone is not sufficient to induce HR despite the N gene elicitor being mapped to the replicase gene of TMV (Padgett and Beachy, 1993). Therefore, the process of virus movement may trigger hypersensitivity, which implicates intercellular signaling in this type of HR. Support for this position comes from experiments in which cell-to-cell contacts were disrupted in N gene tobacco which resulted in the prevention of necrotic lesion formation in infected leaves (Gulyas and Farkas, 1978). Likewise, TMV will not induce HR cell death in NN tobacco protoplasts where plasmodesmata are not intact (Otsuki et al., 1972), although HR does occur in callus cultures where plasmodesmata are intact (Beachy and Murakishi, 1971). By comparison in C. quinoa, movement defective brome mosaic bromovirus (BMV) replicates but fails to move from cell to cell. Initial infection is not sufficient to induce HR since local lesions do not form (Schmitz and Rao, 1996). Similarly, in C. amaranticolor, cucumber mosaic cucumovirus (CMV) lacking a movement protein replicates within inoculated cells, fails to move and does not elicit cell death (Canto and Palukaitis, 1999). Therefore, like TMV on N gene tobacco, the process of viral spread of BMV and CMV in C. quinoa and C. amaranticolor may induce HR.

Methods and Materials

Inoculation of Plants

Leaves of 10-week old C. amaranticolor or C. quinoa were inoculated with in vitro transcripts of TMV-MGfus (Heinlein et al., 1995), TMV virions, tobacco rattle tobravirus (TRV), or they were mock-inoculated. TMV-MGfus encodes GFP (green fluorescent protein) fused to the viral movement protein. Infectious spread can be monitored through the detection of GFP. Using an Olympus stereomicroscope fitted with a U-ULH Olympus lamp, infected C. amaranticolor tissue accumulating GFP was excised at 4, 7 and 11 days after inoculation (dai). Leaves inoculated with TRV or TMV were collected at 4 dai, at which point local lesions were forming. Mock-inoculated tissue was collected at the same time. Tissue was frozen in liquid nitrogen and total RNA was purified from it. Three separate sets of plants were inoculated with TMV-MGfus and yielded three independent preparations of RNA.

cDNA-AFLP (Complementary DNA-Amplified Fragment Length Polymorphism)

Poly-A+ RNA was isolated from TMV-MGfus infected C. amaranticolor using Qiagen's Oligotex mRNA purification system (Qiagen, Valencia, Calif.) and cDNA was generated using cDNA synthesis reagents from Life Technologies (Rockville, Md.). cDNA was used to generate AFLP fragments with the AFLP reagents from Life Technologies and reactions were performed according to the manufacturer's instructions. cDNA made from one microgram of poly-A+ RNA was digested with EcoRI and MseI and the supplied compatible linkers were ligated to the ends of the digested molecules. A few modifications were introduced. EcoRI-NN primers (GACTGCGTACCAATTCNN; SEQ ID NO:2134), rather than EcoRI-NNN, were used with the 5′ fluorescent label NED (Applied Biosystems, Foster City, Calif.) and MseI-N and MseI-NN [GATGAGTCCTGAGTAAN(N); SEQ ID NO:2135), rather than MseI-NNN, primers were used (Genosys, The Woodlands, Tex.), to reduce the complexity of the primer sets evaluated. All possible primer combinations (256+64) were used for PCR amplification and products were separated on polyacrylamide gels and visualized using a Genomyx SC fluorescent scanner (Beckman Coulter, Fullerton, Calif.). Gene fragments that appeared to be upregulated in infected tissues compared to mock-inoculated tissues were tested to see if they were also upregulated by the same primers from a second preparation of cDNA from RNA from a second set of infected plants. Gene fragments that were upregulated in both RNA preparations were excised from the gel, eluted from the gel in water and reamplified by PCR using the appropriate MseI and EcoRI primers and sequenced with 377 ABI sequencers (Applied Biosystems) using dideoxysequencing methods.

Quantitative RT-PCR

DNase treated total RNA (2 ng per reaction) from the third independent preparation of TMV-MGfus infected C. amaranticolor, the first preparation of TRV infected C. amaranticolor, or the first preparation of TMV C. quinoa, was used with TaqMan One-Step RT-PCR reagents for quantitative analysis in an ABI 7700 (Applied Biosystems). Reactions were performed according to the manufacturer's instructions. Primers and 6-FAM 5′ end-labeled probes (6-carboxyfluorescein, Applied Biosystems or Genosys) were designed from the sequences from the C. amaranticolor upregulated gene fragments using Primer Express software (Applied Biosystems) and are listed in SEQ ID Nos:954-1000 and 2130-2135. Expression levels were interpolated from standard curves with a correlation coefficient of 0.99 or greater and the quantities were normalized to the expression level of actin in each sample.

Results

The interaction of the elicitor and the R gene product establishes a cascade of reactions and signaling events that is then manifested in a phenotypic HR. In essence, HR is the end result of disease activated signaling events. In order to detect the early expression of genes induced by viral infection, it was necessary to isolate—infected tissue before the onset of local lesion formation. Therefore, C. amaranticolor was infected with RNA transcripts of TMV-MGfus that express GFP (green fluorescent protein) in infected cells. This allowed the spread of viral infection to be monitored over time. Infection foci comprising over 100 cells could be detected at 4 dai and foci of more than 500 cells could be detected at 7 dai. There was no visible appearances of cell death or chlorotic local lesion formation at the infection foci at 4 and 7 dai. By 11 dai, the infection foci were associated with chlorotic local lesions. Virus infected tissue was excised from leaves at each time point and RNA was purified from the tissue and used for cDNA-AFLP as previously described.

cDNA-AFLP fragments were separated on polyacrylamide sequencing gels and imaged with a fluorescent scanner. Samples derived from mock-inoculated tissue at 7 dai were run next to samples derived from TMV-MGfus infected tissue at 7 dai for comparison. Ninety-eight bands having intensity in the TMV-MGfus lanes that was greater than that of analogous bands in the mock lanes were easily detected. Thirty out of the 98 bands were also upregulated in an independent set of experiments designed to reduce biological variation between experiments. These bands were excised from the gel, reamplified, and sequenced.

The hypothetical protein sequences derived from the reamplified fragments (Seq ID NOs: 1954-1966) translated from all six reading frames were compared to sequences in the GenBank protein sequence database. The results of the BLASTX search (Altschul et al., 1997) are summarized in Table 20a. To confirm that the expression levels of DESCA genes were upregulated in infected tissue compared to mock inoculated tissue, the relative amount of DESCA and actin transcript in a third independent set of samples at 4 dai, 7dai, and 11 dai was quantitatively measured (Table 20b).

The expression level of DESCA1 increased the most in the TMV-MGfus infected plants. The expression level of DESCA1 increased 200 times by 4 dai but tapered off drastically by 11 dai. DESCA1 is unrelated to any protein known at this time.

Two sequences, DESCA4 and DESCA10, are both related to pumps found in Arabidopsis and yeast (Sanchez-Fernandez et al., 1998; Smart and Fleming, 1996). DESCA4 is expressed highly at 4 dai but the expression drops off over time whereas DESCA10 is only moderately induced and its expression returns to normal by the time of the visible appearance of local lesions in C. amaranticolor.

DESCA7 is similar to a salicylate-induced glucosyltransferase gene in tobacco (Horvath and Chua, 1996). DESCA9 is similar to cytochrome P450-like proteins which can produce cytotoxic compounds including phytoalexins that are deployed by a plant to defend against invading microbes. DESCA12 is related to a proanthranilate benzoyltransferase from carnation that plays a direct role in the phytoalexin biosynthesis in carnation (Yang et al., 1998). DESCA11 is similar to the tryptophan biosynthetic enzyme phosphoribosylanthranilate transferase whose gene expression is induced in the presence of ozone in Arabidopsis (Conklin and Last, 1995).

DESCA3 is similar to endo-1,4-betaglucanases that have a role in fruit ripening, abscission, and cell elongation (Lashbrook et al., 1994). DESCA3 is highly expressed in the infected C. amaranticolor and remains highly expressed during the appearance of local lesions and necrosis.

Many disease responses are mediated by positive regulators such as transcription factors or kinases that initiate signaling cascades for the activation of defense responses. One gene, DESCA5, is loosely similar to a yeast potential transcriptional regulator. DESCA5 expression is twice as high at the early stages of infection compared to the late stages of infection illustrating an important role played by gene regulation at the early stages of infection. DESCA6 is related to kinases of Arabidopsis. Kinases have essential roles in programmed cell death during viral infection (Dunigan and Madlener, 1995). DESCA2 is the most highly expressed of the group suggesting that it is an important regulator at the onset of infection. It is similar to a receptor-like protein kinase in bean that responds to Fusarium solani attack (Lange et al., 1999).

Some R genes have kinase-like regions that may function in initiating a signal cascade during the onset of HR (Song et al.; 1995, Zhou et al., 1997). Global amino acid sequence alignment (Henikoff and Henikoff, 1992) of DESCA2 with Pto or Xa21, R genes with ser/thr kinase domains, reveals a 37% similarity. DESCA8 has a nucleotide binding site and a leucine-rich repeat that is common for many R genes. (Meyers et al., 1999; Leister et al., 1998).

To link DESCA genes to a multivirus resistance pathway, C. amaranticolor was inoculated with TRV (tobacco rattle virus), a virus that is taxonomically distinct from TMV. Local lesions appeared by 4 dai and RNA was purified from the infected leaves. DESCA gene expression levels in infected tissue were compared to mock inoculated tissue by quantitative RT-PCR and revealed that the same DESCA genes upregulated during a TMV infection are also upregulated during a TRV infection (Table 20b).

The gene expression levels in TMV infected C. quinoa were measured using the same C. amaranticolor-derived primers in quantitative PCR to determine if DESCA genes were up-regulated during HR in another Chenopodium species. Most of the DESCA genes were upregulated in C. quinoa and were expressed at levels many times higher than in C. amaranticolor (Table 20b). This may be a result of the infection of C. quinoa with the aggressive wild-type virus rather than slower moving TMV-MGfus.

The experimental procedure presented here can detect any similar gene involved in the aforementioned signaling pathways such as SA signaling. Except for DESCA 1, whose expression is increased the most at 200+ fold, many of the fragments have homology to other genes that have been placed in disease resistance pathways in other plants. DESCA12 and DESCA9 are respectively similar to hypersensitivity related gene 201, possibly a proanthranilate benzoyltranferase, and p450 monooxygenases, both which are expressed during the hypersensitive response in tobacco upon infection with Pseudomonas solanacearum but are not regulated by SA (Czemic et al., 1996). DESCA7 is similar to a salicylate-induced glucosyltransferase gene in tobacco (Horvath and Chua, 1996). Thus, the disease resistance response in C. amaranticolor involves pathways both dependent and independent of SA signaling.

The surprising discovery of DESCA4, DESCA7, DESCA9, DESCA10, and DESCA12, reveal the underpinnings of an endogenous detoxification system. Briefly, the activation phase involves cytochrome P450 monooxygenases introducing functional groups (e.g. aromatic rings) to potential toxins. The conjugation phase in plants involves the linking of glutathione or glucose to the toxin at which point the conjugated molecule can be recognized by an ATP-binding cassette transporter and pumped into the vacuole, or possibly the neighboring cells, during the elimination phase. The final phase includes either storage or breakdown of such molecules. DESCA9, similar to cytochrome P450, and DESCA12, similar to a gene associated with the production of phytoalexin, may produce potential toxins. In fact, C. amaranticolor produces many such compounds that are antiviral to TMV. DESCA7, similar to a glucosyltransferase, may conjugate such toxins to be transported by the ABC-transporters encoded by DESCA4 or DESCA 10. In this particular case, the transported compound could then be deployed by the infected plant cell as an antiviral agent or cytotoxic compound, stored by noninfected cells in anticipation of infection, or eliminated by noninfected cells neighboring infected cells. Since all of these genes are induced by TMV and TRV in C. amaranticolor, their induced expressions are a result of a specific or general multivirus or disease resistance pathway.

Possessing the R genes that allow C. amaranticolor to initially recognize multiple viruses provides an opportunity to use these genes, and the regulatory elements associated with these genes, to transfer viral resistance to other plants. In addition, possession of genes that produce and transport antiviral and cytotoxic products allows for the transfer of viral resistance through a mechanism involving induced cell death upon viral infection.

Two genes that may be used for early recognition of viral infection are DESCA8 and DESCA2, as these genes may act as signaling components to initiate the resistance cascade. DESCA8 has a nucleotide binding site and a leucine-rich repeat that is common for many R genes and that can be found in other plants (Meyers et al., 1999; Leister et al., 1998). DESCA2 is induced in both Chenopodium species and is similar to other R genes, Xa21 and Pto, which have similar ser/thr kinase domains.

Resistance to viral spread may be transferred between Chenopodium spp. For example, BMV (brome mosaic virus) induces local lesions in the green variety of C. hybridum, however lesion formation does not limit the systemic spread of the virus (Verduin, 1978). The systematic spread of the BMV virus may be restricted in the green variety of C. hybridum by transformation with a gene from the purple variety that does limit spread (Komari, 1990). Thus, genes that confer viral resistance may be used for complementation, reverse genetics, overexpression, and gene silencing. Furthermore, as indicated by the functionality of the R genes N and Pto after being transferred into heterologous species, (Whitham et al., 1996; Rommens et al., 1995), the Chenopodium genes may function to initiate hypersensitivity in crops, Arabidopsis or other useful plants.

### EXAMPLE 8

### Other Plant-Pathogen Interactions

The methods set out hereinabove can be used for any type of comparable resistance interaction. For example any of the following plant/pathogen interactions will be produced as compatible and incompatible interactions. The RNA from such an interaction is isolated and subject to a protocol such as one outlined in Example 1, e.g., using a Genechip with a specific plant's genes or microarray, differential display PCR or cDNA-ALFP (Example 7). A four-way analysis is performed and genes which are expressed differently are identified. The plant/pathogen interactions in Table 21 are well known in the art. However, any type of plant/pathogen interaction that involves this type of resistance can be used.

TABLE 21

| Plant | Pathogen |
|---|---|
| Tomato | Cladosporium fulvum |
| Maize | Rust fungus |
| Antirrhinium | Rust fungus |
| Flax | Melampsora lini |
| Lettuce | Downy mildew |
| Arabidopsis | Peronospora parasitica |
| Tomato | Nematode |
| Corn | Cochliobolus carbonum |
| Tomato | Pseudomonas syringae |
| Rice | Xanthomonas oryzae pv. Oryzae |
| Rice | Pyricularia oryzae |
| Tobacco | Tobacco Mosaic Virus |

Genes that are upregulated and cause resistance in a wide variety of plants are particularly useful in methods which upregulate or overexpress the gene. One method is to add an exogenous copy, thus providing more of the gene product or allowing for a different induction from that used by the plant. Alternatively, the endogenous gene can be upregulated using a known inducer or using artificial methods such as using an artificial induction signal in the endogenous promoter. Examples of the two methods are provided in Examples 9 and 10.

Accordingly, embodiments of the invention provide the sequences disclosed herein, which sequences can be used in genetic engineering of crops, as probes and markers to study the dynamics of plant/pathogen interactions, and as markers in marker-assisted breeding protocols to identify plants carrying particularly useful combinations of genes associated with pathogen resistance, as well as in plant defense.

### EXAMPLE 9

### Transformation of Resistance Genes into Plants

To produce resistant plants, resistance genes such as those identified herein can be introduced into plant cells to generate transgenic plants having enhanced resistance. While HSF4 is any preferred gene for this embodiment of the invention, the invention can be employed with other genes, alone or in combination, whose regulation is strongly responsive to plant/pathogen interactions, such as the genes identified herein. Since some genes are strongly induced and others are strongly repressed in plant/pathogen interactions, and since some genes that are strongly induced in one ecotype can be strongly repressed in another, the invention contemplates use of any of the genes and sequences, or fragments thereof, disclosed herein, in a construct adapted to cause overexpression, repression, or knock out, of the genes in a transgenic plant.

Transgenic downregulation of genes associated with pathogen resistance can have several useful applications. In one embodiment, transgenic downregulation of genes that are strongly repressed in resistance interactions can enhance resistance. Such transgenic downregulation can employ the genes disclosed herein, or fragments thereof, in an antisense orientation to interfere with accumulation of the products of those genes. Likewise, any other methodology capable of lowering expression of such genes is also included within these embodiments of the invention.

Plant transformation can be carried out by conventional means, and can include Agrobacterium-mediated transformation, electroporation, particle acceleration, abrasion, and any other useful means leading to expression of a transgene in a plant of interest. Transformed plant cell are then used to regenerate one or more plants in tissue culture. Subsequent generations of transgenic plants can be used directly or bred with other lines to generate plants having enhanced pathogen resistance.

### EXAMPLE 10

### Upregulation of Resistance Genes in Crops

Because many or most Arabidopsis genes have orthologs in other plants, the genes and sequences disclosed herein are generally useful in constructs to be up-regulated and cause resistance in a wide variety of plants. As examples, the heat shock proteins, and particularly HSF4, are found throughout the plant kingdom. For many such regulatory and responsive genes it is well known that there exist substances that can induce expression. Chemicals such as dexamethasone have been found to induce mammalian HSF proteins. Likewise, a chemical induction of key plant defense genes can be chemically induced. High throughput screening for chemical inducers of the plant HSF4 or other resistance gene is performed. Potentially useful substances are then tested on crop plants and eventually used as a soil additive or sprayed onto plants when needed to induce resistance. Accordingly, embodiments of the invention usefully employ the genes disclosed herein, or fragments thereof, for screening to identify useful chemical inducers and/or repressors of gene responsive to pathogenic infections.

### EXAMPLE 11

### Identification of Inducers and Repressors of Resistance Genes

The yeast two-hybrid method and many methods which use its basic idea, provide a technique to identify proteins which interact with a protein of interest. The method relies on the fact that a protein contains domains which can be separated. Thus the protein of interest is fused to the GAL4 DNA binding region of a known protein. The GAL4 (or another) activation signal is fused in a library to produce a library of fused proteins. If one of the proteins from the library interacts with the protein of interest the protein binds and a signal protein is produced, such as luciferase. There are a number of such systems presently, some of which can be used in mammalian cells, allowing for correct processing and folding of certain proteins and others which allow the interaction to occur in the cytoplasm allowing for the identification of other types of proteins.

cDNA from HSF4 and any other protein of interest is cloned in fusion to the yeast GAL4 DNA binding domain on a vector. A library containing cDNA from Arabidopsis is fused to the GAL4 or an activation domain of choice. Expression of luciferase correlates with identification of an interacting protein. This protein is then analyzed as to its action as an inducer or repressor.

### EXAMPLE 12

### Determination of the Minimal Promoter Fragment

The full-length promoter sequence as given in SEQ ID Nos: 1001-1095, 2137-2661 and 4738-6813, or the promoter orthologs thereof is fused to the β-glucuronidase (GUS) gene at the native ATG to obtain a chimeric gene cloned into plasmid DNA. The plasmid DNA is then digested with restriction enzymes to release a fragment comprising the full-length promoter sequence and the GUS gene, which is then used to construct the binary vector. This binary vector is transformed into Agrobacterium tumefaciens, which is in turn used to transform Arabidopsis plants (for further details of the binary vector construction see above Example 9).

The above plasmid can also be used to form a series of 5′ end deletion mutants having increasingly shorter promoter fragments fused to the GUS gene at the native ATG. Various restriction enzymes are used to digest the plasmid DNA to obtain the binary vectors with different lengths of promoter fragments. In particular, a binary vector 1 is constructed with a 1,900-bp long promoter fragment; a binary vector 2 is constructed with a 1,300-bp long promoter fragment; a binary vector 3 is constructed with a 1000-bp long promoter fragment; a binary vector 4 is constructed with a 800-bp long promoter fragment; a binary vector 5 is constructed with a 700-bp long promoter fragment; a binary vector 6 is constructed with a 600-bp long promoter fragment; a binary vector 6 is constructed with a 500-bp long promoter fragment; and a binary vector 7 is constructed with a 100-bp long promoter fragment. Like the binary vector comprising the full-length promoter fragment, these 5′ end deletion mutants are also transformed into Agrobacterium tumefaciens and, in turn, Arabidopsis plants (for further details of Arbabidopsis transformation and promoter assay procedures see Example 5 above).

The presence of the correct hybrid construct in the transgenic lines is confirmed by PCR amplification.

By using the above protocol it can be determined, which portion of the promoter sequences given in SEQ ID Nos: 1001-1095, 2137-2661 and 4738-6813, or the promoter orthologs thereof is required for gene expression.

Minimal promoter fragments having lengths substantially less than the full-length promoter can therefore be operatively linked to coding sequences to form smaller constructs than can be formed using the full-length promoter. As noted earlier, shorter DNA fragments are often more amenable to manipulation than longer fragments. The chimeric gene constructs thus formed can then be transformed into hosts such as crop plants to enable at-will regulation of coding sequences in the hosts.

### EXAMPLE 13

### Determination of Promoter Motifs

While a deletion analysis characterizes regions in a promoter that are required overall for its regulation, linker-scanning mutagenesis allows for the identification of short defined motifs whose mutation alters the promoter activity. Accordingly, a set of linker-scanning mutant promoters fused to the coding sequence of the GUS reporter gene are constructed. Each of them contains a 8-10-bp mutation located between defined positions and included in a promoter fragment as given in SEQ ID Nos: 1001-1095, 2137-2661 and 4738-6813, or the promoter orthologs thereof.

Each construct is transformed into Arabidopsis and GUS activity is assayed for 19 to 30 independent transgenic lines. The presence of the correct hybrid consstruct in transgenic lines is confirmed by PCR amplification of all lines containing the mutant constructs and by random sampling of lines containing the other constructs. Amplified fragments are digested with restriction enzyme (e.g. XbaI) and separated on high resolution agarose gels to distinguish between the different mutant constructs. constructs. The effect of each mutation on promoter activity is compared to an equivalent number of transgenic lines containing the unmutated construct. Two repetitions resulting from independent plating of seeds are carried out in every case.

The sequences mutated in the linker-scanning constructs, in particular those that showed marked differences from the control construct, are then examined more closely.

### EXAMPLE 14

### Identifying Orthologs

Orthologs were identified through use of BLAST and SCAN software with some additional filters. For the Arabidopsis search, a BLAST database was created that was a subset of GenBank ver 123.0 (released Apr. 15, 2001) that contained all of the plant translated regions excluding Arabidopsis thaliana sequences. The subset was created with PERL script. A BLAST search with all of the peptide sequences was performed against the GenBank subset. Each query was executed using the “blastall” command with the parameters” “-p blastp”, “-v 50”, “-b 50”, “-F F”. The BLAST search results were then processed with SCAN (Sequence Comparison Analysis program, version 1.0k, Los Alamos National Laboratories) using default settings and the orthologs were identified following implementation of an E-value cutoff of <=1e-4. The candidate orthologs were further filtered by comparing words in the description to the text of the annotation fields: product, function and note. The sequence was considered to have the same or similar function if any of the words matched. Words excluded from the filter included: the, like, protein, related, unknown, subunit, hypothetical, and, putative, precursor, clone, homolog, small, beta, class, dna, ma, alpha, gamma, has, not, been, from, to, by, long, type and induced.

For the rice search, amino acid sequences were used that resulted from FGENESH (version 1.C) gene prediction results. The peptide sequences were obtained from gene predictions and formatted into a BLAST database. A BLASTP comparison was then performed against the Arabidopsis sequences. The BLASTP results were then filtered through use of SCAN with the following parameters: “-a 60 60” with an E-value cutoff of 1e-4. This produced orthologs having 60 or more identities and where 60% of the alignments were made up of identities.

The following pages compile Tables 3 to 20 referred to in the examples above.

LENGTHY TABLE REFERENCED HERE
US20070016976A1-20070118-T00001
Please refer to the end of the specification for access instructions.

LENGTHY TABLE REFERENCED HERE
US20070016976A1-20070118-T00002
Please refer to the end of the specification for access instructions.

LENGTHY TABLE REFERENCED HERE
US20070016976A1-20070118-T00003
Please refer to the end of the specification for access instructions.

LENGTHY TABLE REFERENCED HERE
US20070016976A1-20070118-T00004
Please refer to the end of the specification for access instructions.

LENGTHY TABLE REFERENCED HERE
US20070016976A1-20070118-T00005
Please refer to the end of the specification for access instructions.

LENGTHY TABLE REFERENCED HERE
US20070016976A1-20070118-T00006
Please refer to the end of the specification for access instructions.

LENGTHY TABLE REFERENCED HERE
US20070016976A1-20070118-T00007
Please refer to the end of the specification for access instructions.

LENGTHY TABLE REFERENCED HERE
US20070016976A1-20070118-T00008
Please refer to the end of the specification for access instructions.

LENGTHY TABLE REFERENCED HERE
US20070016976A1-20070118-T00009
Please refer to the end of the specification for access instructions.

LENGTHY TABLE REFERENCED HERE
US20070016976A1-20070118-T00010
Please refer to the end of the specification for access instructions.

LENGTHY TABLE REFERENCED HERE
US20070016976A1-20070118-T00011
Please refer to the end of the specification for access instructions.

LENGTHY TABLE REFERENCED HERE
US20070016976A1-20070118-T00012
Please refer to the end of the specification for access instructions.

LENGTHY TABLE REFERENCED HERE
US20070016976A1-20070118-T00013
Please refer to the end of the specification for access instructions.

LENGTHY TABLE REFERENCED HERE
US20070016976A1-20070118-T00014
Please refer to the end of the specification for access instructions.

LENGTHY TABLE REFERENCED HERE
US20070016976A1-20070118-T00015
Please refer to the end of the specification for access instructions.

LENGTHY TABLE REFERENCED HERE
US20070016976A1-20070118-T00016
Please refer to the end of the specification for access instructions.

LENGTHY TABLE REFERENCED HERE
US20070016976A1-20070118-T00017
Please refer to the end of the specification for access instructions.

LENGTHY TABLE REFERENCED HERE
US20070016976A1-20070118-T00018
Please refer to the end of the specification for access instructions.

LENGTHY TABLE REFERENCED HERE
US20070016976A1-20070118-T00019
Please refer to the end of the specification for access instructions.

LENGTHY TABLE REFERENCED HERE
US20070016976A1-20070118-T00020
Please refer to the end of the specification for access instructions.

LENGTHY TABLE REFERENCED HERE
US20070016976A1-20070118-T00021
Please refer to the end of the specification for access instructions.

LENGTHY TABLE REFERENCED HERE
US20070016976A1-20070118-T00022
Please refer to the end of the specification for access instructions.

LENGTHY TABLE REFERENCED HERE
US20070016976A1-20070118-T00023
Please refer to the end of the specification for access instructions.

LENGTHY TABLE REFERENCED HERE
US20070016976A1-20070118-T00024
Please refer to the end of the specification for access instructions.

LENGTHY TABLE REFERENCED HERE
US20070016976A1-20070118-T00025
Please refer to the end of the specification for access instructions.

LENGTHY TABLE REFERENCED HERE
US20070016976A1-20070118-T00026
Please refer to the end of the specification for access instructions.

LENGTHY TABLE REFERENCED HERE
US20070016976A1-20070118-T00027
Please refer to the end of the specification for access instructions.

LENGTHY TABLE REFERENCED HERE
US20070016976A1-20070118-T00028
Please refer to the end of the specification for access instructions.

LENGTHY TABLE REFERENCED HERE
US20070016976A1-20070118-T00029
Please refer to the end of the specification for access instructions.

The material on the CD-ROM (filed in duplicate herewith; CD 010623_{—}1108, file 38236 created on Jun. 23, 2001, size 12,203 KB) is hereby incorporated by reference herein pursuant to 37 C.F.R. sec. 1.52(e)(5).

### REFERENCES

Aarts et al., Proc Natl Acad Sci 95:10306 (1998).
Alonso et al., Science, 284:2148 (1999)
Altschul et al., J. Mol. Biol. 215:403 (1990).
Altschul et al., Nucleic Acids Res. 25:3389 (1997).
An et al., EMBO J. 4:277 (1985).
Aoyama et al., N-H Plant Journal, 11:605 (1997).
Bachem et al., Plant J., 9:745 (1996).
Bailey and Elkan In: Proceedings of the second international Conference on Intelligent Systems for Molecular Biology (Altman, R., ed), pp 28-36, AAAI press (1994).
Ballas et al., Nucleic Acids Res. 17:7891 (1989).
Balzi et al., J. Biol. Chem., 269:2206 (1994).
Bansal et al., Proc. Natl. Acad. Sci. USA, 89:3654 (1992).
Batzer et al., Nucleic Acid Res., 19:5081 (1991).
Beachy and Murakishi, Phytopathology, 61:877 (1971).
Beals et al., Plant Cell, 9:1527 (1997).
Belanger et al., Genetics, 129:863 (1991).
Bevan et al., Nature, 304:184 (1983).
Blochinger & Diggelmann, Mol Cell Biol, 4:2929.
Bourouis et al., EMBO J., 2:1099 (1983).
Boyes et al., Proc Natl Acad Sci USA, 95:15849 (1998).
Byrne et al. Plant Cell Tissue and Organ Culture, 8:3 (1987).
Campbell and Gowri, Plant Physiol., 92:1 (1990).
Canto and Palukaitis, Virology, 265:74 (1999).
Cao et al., Plant Cell, 6:1583 (1994).
Cao et al., Cell, 88:57 (1997).
Century et al., Science, 278:1963 (1997).
Chandler et al., Plant Cell, 1:1175 (1989).
Christou et al., Biotechnology, 9:957 (1991).
Christou et al., Plant Physiol., 87:671 (1988).
Conklin and Last, Plant Physiol., 109:203 (1995).
Cooper et al., Virology, 206:307 (1995)
Cooper et al., Virology, 216:208 (1996).
Cordero et al., Plant J., 6:141 (1994).
Corpet et al., Nucleic Acids Res. 16:10881 (1988).
Crameri et al., Nature Biotech., 15:436 (1997).
Crameri et al., Nature, 391:288 (1998).
Creelman et al., Annu Rev Plant Physiol Plant Mol Biol 48:355 (1997).
Crossway et al., BioTechniques, 4:320 (1986).
Czako et al., Mol. Gen. Genet. 23 5 (1), 33-40 (1992).
Czemic et al., Plant Mol. Biol., 31:255 (1996).
Datta et al., Bio/Technology, 8, 736 (1990).
Dayhoff et al., Atlas of Protein Sequence and Structure, Natl. Biomed. Res. Found., Washington, C.D. (1978).
De Blaere et al., Meth. Enzymol. 143:277 (1987).
de Framond, FEBS, 290:103 (1991).
Delaney et al., Proc. Natl. Acad. Sci. USA 92:6602 (1995).
Della-Cioppa et al., Plant Physiology, 84:965 (1987).
De Oliveira et al., Microbios., 76:213 (1993).
Dennis et al., Nucleic Acids Res., 12:3983 (1984).
Dong et al., Curr Opin Plant Biol 1:316 (1998).
Dunigan and Madlener, Virology, 207:460 (1995).
Durrant et al., The Plant Cell, 12:963 (2000).
Dzelkalns et al., Plant Cell, 5:855 (1993).
Eisen et al., Trends in Plant Sci. 95:14863 (2000).
Ellis and Jones, Curr Opin Plant Bio 1:288 (1998).
Elroy-Stein et al., PNAS USA, 86:6126 (1989).
English, et al., Plant Cell 8:179 (1996).
Eulgem et al., Trends in Plant Sci. 5:199 (2000).
Falk et al., Proc Natl Acad Sci USA 96:3292 (1999).
Feys et al., Plant Cell 6:751 (1994).
Franken et al., EMBO J., 10:2605 (1991).
Fromm et al., Bio/Technology 8:833 (1990).
Gallie et al., Molecular Biology of RNA, 237 (1989).
Gallie et al., Nucl. Acids Res., 15:8693 (1987).
Gan et al., Science (1995), 270 (5244), 1986-8).
Gatz Current Opinion in Biotechnology, 7:168 (1996).
Gatz, C., Annu. Rev. Plant Physiol. Plant Mol. Biol., 48:89 (1997).
Gelfand, eds., PCR Strategies (Academic Press, New York (1995)).
Glazebrook, J., Curr. Opin. Plat Biology, 2:280 (1999).
Glazebrook et al., Genetics 143:973 (1996).
Glazebrook et al., Proc. Natl. Acad. Sci. USA 91:8955 (1994).
Gordon-Kamm et al., Plant Cell, 2, 603 (1990).
Graham et al., Biochem. Biophys. Res. Comm., 101:1164 (1981).
Graham et al., J. Biol. Chem., 260:6555 (1985).
Graham et al., J. Biol. Chem., 260:6561 (1985).
Guerineau et al., Mol. Gen. Genet. 262:141 (1991).
Gulyas and Farkas, Phytopath. Z., 91:182 (1978)
Hammand-Kosack and Jones, Ann. Rev. Plant Physiol. Plant Mol. Biol., 48:575 1997).
Heinlein et al., Science, 270:1983 (1995).
Henikoff & Henikoff, Proc. Natl. Acad. Sci. USA 89:10915 (1989).
Henikoff and Henikoff, Proc. Natl. Acad. Sci. USA, 89:10915 (1992).
Hiei et al., Plant J. 6:271 (1994).
Higgins et al., Gene 73:237 (1988).
Higgins et al., CABIOS 5:151 (1989).
Hinchee et al., Biotechnology, 6:915 (1988).
Hoekema, In: The Binary Plant Vector System. Offset-drukkerij Kanters B. V.
Horvath and Chua, Plant Mol. Biol., 31:1061 (1996).
Huang et al., CABIOS 8:155 (1992).
Hudspeth & Grula, Plant Molec. Biol. 12:579 (1989).
Huffman et al., J. Cell. Biochem., 17B: Abstract.
Hunt et al., Mol. Plant-Microbe Int. 9:261 (1998).
Ingelbrecht et al., Plant Cell, 1:671 (1989).
Innis et al., eds., PCR Protocols: A Guide to Methods and Applications (Academic Press, New York (1995).
Innis and Gelfand, eds., PCR Methods Manual (Academic Press, New York) (1999).
Jobling et al., Nature, 325:622 (1987).
John et al., Proc. Natl. Acad. Sci. USA 89(13):5769 (1992).
Jones et al., Adv Bot Res 24:89 (1997).
Joshi et al., Nucleic Acid Res. 15:9627 (1987).
Joshi, Nucl. Acid Res., 15, 6643 (197).
Karlin and Altschul, Proc. Natl. Acad. Sci. USA 87:2264 (1990).
Karlin and Altschul, Proc. Natl. Acad. Sci. USA 90:5873 (1993).
Keller et al., Genes Dev., 3:1639 (1989).
Klein et al., Bio/Technology, 6:559 (1988).
Klein et al., Nature (London) 327:70 (1987).
Klein et al., Plant Physiol., 91:440 (1988).
Klein et al., Proc. Natl. Acad. Sci. USA, 85:4305 (1988).
Kohler et al., Plant Mol. Biol., 29:1293 (1995).
Knauf, et al., Genetic Analysis of Host Range Expression by Agrobacterium In: Molecular Genetics of the Bacteria-Plant Interaction, Puhler, A. ed., Springer-Verlag, New York, 245 (1983).
Komari, Plant Cell Reports, 9:303 (1990).
Koziel et al., Biotechnology, 11:194 (1993).
Kridl et al., Seed Science Research, 1:209 (1991).
Kriz et al., Mol. Gen. Genet., 207:90 (1987).
Kunkel, Proc. Natl. Acad. Sci. USA, 82:488 (1985).
Kunkel et al., Methods in Enzymol., 154:367 (1987).
Lange et al., Plant Sci., 142:133 (1999).
Langridge et al., Cell, 34:1015 (1983).
Lashbrook et al., Plant Cell 6:1485 (1994).
Leister et al., P.N.A.S., (USA), 95:370 (1998).
Leister et al., Proc. Natl. Acad. Sci. USA, 95:370 (1998).
Liang et al., Science, 257:967 (1992).
Lindstrom et al., Der. Genet., 11: 160 (1990).
Lommel et al., Virology, 81:382 (1991).
Ly et al., Science, 287:2486 (2000).
Macejak et al., Nature, 353:90 (1991).
Mansson et al., Gen. Genet., 200:356 (1985).
Martin and Paz-Ares, Trends in Genetics 13:67 (1997).
Martinez et al., J. Mol. Biol., 208:551 (1989).
McBride et al., Proc. Natl. Acad. Sci. USA, 91:7301 (1994).
McCabe et al., Bio/Technology, 6:923 (1988).
McDowell et al., Plant J. 22:523 (2000).
McDowell et al., Plant Cell 10:1861 (1998).
McNellis et al., Plant J. 1998, 14:247-257.
Meinkoth and Wahl, Anal. Biochem. 138:267 (1984).
Messing & Vierra, Gene, 19:259 (1982).
Meyers et al., Plant J. 20:317 (1999).
Mogen et al., Plant Cell 2:1261 (1990).
Moore et al., J. Mol. Biol., 272:336 (1997).
Munroe et al., Gene 91:151 (1990).
Murray et al., Nucleic Acids Res., 17:477 (1989).
Myers and Miller, CABIOS 4:11 (1988).
Needleman and Wunsch, J. Mol. Biol. 48:443 (1970).
Odell et al., Nature, 313:810 (1985).
Odell et al., Nature, 313:810 (1985).
Ohtsuka et al., J. Biol. Chem., 260:2605 (1985).
Okamuro et al., Biochemistry of Plants, 15:1 (1989).
Otsuki et al., Virology, 50:45 (1972)
Paszkowski et al., EMBO J., 3:2717 (1984).
Pacciotti et al. Bio/Technology 3:241 (1985).
Padgett and Beachy, Plant Cell, 5:577 (1993).
Park et al., J. Plant Biol. 38(4):365 (1985).
Pearson et al., Meth. Mol. Biol. 24:307 (1994).
Pearson and Lipman, Proc. Natl. Acad. Sci. 85:2444 (1988).
Penninck et al., Plant Cell 8:2309 (1996).
Perlak et al., Proc. Natl. Acad. Sci. USA, 88:3324 (1991).
Pieterse et al., Plant Cell, 10:1571 (1998).
Pieterse et al., Plant Cell, 8:1225 (1996).
Proudfoot, Cell 64:671 (1991).
Quigley et al., J. Mol. Evol., 29:412 (1989).
Ralston et al., Genetics, 119:185 (1988).
Reina et al., Nucleic Acids Res., 18:6425 (1990).
Reina et al., Nucleic Acids Res., 18:7449 (1990).
Riggs et al., Proc. Natl. Acad. Sci. USA, 83:5602 (1986).
Rochester et al., (1986).
Rommens et al., Plant Cell, 7:1537 (1995).
Ronald, Curr Opin Plant Biol 1:294 (1998).
Rossolini et al., Mol. Cell. Probes, 8:91 (1994).
Roth et al., Nature Biotechnology, 16:939 (1998).
Ruiz et al., Plant Cell 10:937 (1998).
Ryals et al., Plant Cell 8:1809 (1996).
Ryals et al., Plant Cell 9:425 (1997)
Sambrook et al., Molecular Cloning: A Laboratory Manual (2d ed., Cold Spring Harbor Laboratory Press, Plainview, N.Y.) (1989).
Sanchez-Fernandez et al., Mol. Gen. Genet., 258:655 (1998).
Sanfacon et al., Genes Dev. 5:141 (1991).
Sanford et al., Particulate Science and Technology, 5:27 (1987).
Schemthaner et al., EMBO J., 7:1249 (1988).
Schindler et al., Plant Cell 4:1309 (1992).
Schmitz and Rao, Virology, 248:323 (1996)
Schwob et al., Plant J., 4:423 (1993).
Shah et al., Mol. Plant-Microbe Interact. 10:69 (1997).
Shimamoto et al., Nature, 338:274 (1989).
Shirasu et al., Plant Cell 9:261 (1997).
Shulaev et al., Plant Cell 7:1691 (1995).
Simpson, Plant Mol. Biol., 19:699 (1985).
Skuzeski et al., Plant Molec. Biol., 15:65 (1990).
Slater et al., Plant Mol. Biol., 5:137 (1985).
Smart and Fleming, J. Biol. Chem., 271:19351 (1996).
Smith et al., Adv. Appl. Math. 2:482 (1981).
Smith et al., Planta 168:94 (1986).
Song et al., Science, 270:1804 (1995).
Spencer et al., Theor Appl Genet, 79:625 (1990).
Staswick et al., Proc. Natl. Acad. Sci. USA 89:6837 (1992).
Staub et al., EMBO J., 12:601 (1993).
Staub et al., Plant Cell, 4:39 (1992).
Stemmer, Nature, 370:389 (1994).
Stemmer, Proc. Natl. Acad. Sci. USA, 91:10747 (1994).
Sukhapinda et al. Plant Mol. Biol. 8:209 (1987).
Sullivan et al., Mol. Gen. Genet., 215:431 (1989).
Svab et al., Proc. Natl. Acad. Sci. USA, 87:8526 (1990).
Svab et al., Proc. Natl. Acad. Sci. USA, 90:913 (1993).
Thomma et al., Plant Physiol., 121:1093 (1999).
Thomma et al., Proc. Natl. Acad. sci. U.S.A., 85:15107 (1998).
Tijssen, Laboratory Techniques in Biochemistry and Molecular Biology-Hybridization with Nucleic Acid Probes, part I chapter 2 “Overview of principles of hybridization and the strategy of nucleic acid probe assays” Elsevier, New York (1993).
Turner et al., Molecular Biotechnology, 3:225 (1995).
VanTunen et al., EMBO J., 7:1257 (1988).
Vasil et al., Biotechnology, 11:1553 (1993).
Verduin, J. Gen. Virol., 38:571 (1978).
Vemooij et al., Plant Cell 6:959 (1994).
Visedo et al., Physiologia Plantarum 78:218 (1990).
Vodkin, Prog. Clin. Biol. Res., 138:87 (1983).
Vogel et al., EMBO J., 11:157 (1992).
Walker and Gaastra, eds., Techniques in Molecular Biology, MacMillan Publishing Company, New York (1983).
Wandelt et al., Nucleic Acids Res., 17:2354 (1989).
Waterman, M. S. Introduction to Computational Biology: Maps, sequences and genomes. Chapman & Hall. London (1995). Weeks et al., Plant Physiol., 102:1077 (1993).
Weissinger et al., Annual Rev. Genet., 22:421 (1988).
Wenzler et al., Plant Mol. Biol., 13:347 (1989).
Weymann et al., Plant Cell 7:2013 (1995).
White et al., Nucl Acids Res, 18:1062 (1990).
Whitham et al., P.N.A.S., (USA), 93:8776 (1996).
Whitham et al., Cell, 78:1101 (1994).
Willits et al., Mol Plant-Microbe Interact 11:795 (1998).
Xie et al., Science 280:1091 (1998).
Yamamoto et al., Nucleic Acids Res., 18:7449 (1990).
Yang et al., Plant Mol. Biol., 38:1201 (1998).
Yang & Klessig, Proc. Natl. Acad. Sci. USA 93:14972 (2000).
Yu et al., Proc. Natl. Acad. Sci. USA 95:7819 (1998).
Zhang et al., Proc. Natl. Acad. Sci. USA, 94:4504 (1997).
Zhou et al., EMBO J., 16:3207 (1997).
Zhou et al., Plant Cell 8:2235 (1996).
Zhou et al., Plant Cell 10:1021 (1998).

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

LENGTHY TABLE
The patent application contains a lengthy table section. A copy of the table is available in electronic form from the USPTO web site (http://seqdata.uspto.gov/?pageRequest=docDetail&DocID=US20070016976A1) An electronic copy of the table will also be available from the USPTO upon request and payment of the fee set forth in 37 CFR 1.19(b)(3).

What is claimed is:

1. An isolated polynucleotide comprising a plant nucleotide sequence that alters transcription of an operatively linked nucleic acid segment in a plant cell after pathogen infection, which plant nucleotide sequence is from a gene encoding a polypeptide that is substantially similar to a polypeptide encoded by a gene comprising a promoter selected from the group consisting of SEQ ID NOs: 1047, 1051, 1053, 4794, 4892, 5261, 5738 and 6469.

2. An isolated polynucleotide comprising a plant nucleotide sequence that alters transcription of an operatively linked nucleic acid segment in a plant cell after pathogen infection, which plant nucleotide sequence hybridizes under high stringency conditions to the complement of any one of SEQ ID NOs:1047, 1051, 1053, 4794, 4892, 5261, 5738 or 6469.

3. The isolated polynucleotide of claim 2, which plant nucleotide sequence hybridizes under very high stringency conditions to the complement of any one of SEQ ID NOs: 1047, 1051, 1053, 4794, 4892, 5261, 5738 or 6469.

4. The isolated polynucleotide of claim 1 or 2 which is selected from the group consisting of SEQ ID NOs: 1047, 1051, 1053, 4794, 4892, 5261, 5738, 6469, and a fragment thereof.

5. The polynucleotide of claim 1 or 2 wherein the plant nucleotide sequence is 25 to 2000 nucleotides in length.

6. The polynucleotide of claim 1 or 2 wherein the plant nucleotide sequence is from a dicot.

7. The polynucleotide of claim 1 or 2 wherein the plant nucleotide sequence is from a monocot.

8. The polynucleotide of claim 1 or 2 wherein the plant nucleotide sequence is from a cereal plant.

9. The polynucleotide of claim 1 or 2 wherein the plant nucleotide sequence is a maize, soybean, barley, alfalfa, sunflower, canola, soybean, cotton, peanut, sorghum, tobacco, sugarbeet, rice or wheat sequence.

10. An expression cassette comprising the polynucleotide of claim 1 or 2 operatively linked to an open reading frame.

11. A host cell comprising the expression cassette of claim 10.

12. The host cell of claim 11 wherein the cell is a yeast, a plant cell, a bacterium, a cereal plant cell, or an Arabidopsis cell.

13. The host cell of claim 11 which is a monocot cell.

14. The host cell of claim 11 which is a dicot cell.

15. A transformed plant, the genome of which is augmented with the expression cassette of claim 10.

16. The transformed plant of claim 15 which is a dicot.

17. The transformed plant of claim 15 which is a monocot.

18. The transformed plant of claim 15 which is selected from the group consisting of maize, soybean, barley, alfalfa, sunflower, canola, soybean, cotton, peanut, sorghum, tobacco, sugarbeet, rice, wheat and Arabidopsis.

19. A method for augmenting a plant genome, comprising:
a) contacting a plant cell with an expression cassette comprising a promoter from a gene encoding a polypeptide that is substantially similar to a polypeptide encoded by a gene comprising a promoter selected from the group consisting of SEQ ID NOs: 1047, 1051, 1053, 4794, 4892, 5261, 5738 and 6469 operatively linked to an open reading frame so as to yield a transformed plant cell; and
b) regenerating the transformed plant cell to provide a differentiated transformed plant, wherein the differentiated transformed plant expresses the open reading frame in the cells of the plant.

20. A method to alter the phenotype of a plant cell comprising: introducing an expression cassette comprising a promoter from a gene encoding a polypeptide that is substantially similar to a polypeptide encoded by a gene comprising a promoter selected from the group consisting of SEQ ID NOs: 1047, 1051, 1053, 4794, 4892, 5261, 5738 and 6469 operatively linked to an open reading frame into the plant cell and expressing the open reading frame in the cell so as to alter a characteristic of that cell relative to a plant cell that does not comprise the expression cassette.

21. The method of claim 19 or 20 wherein the plant cell is a dicot cell.

22. The method of claim 19 or 20 wherein the plant is a monocot cell.

23. The method of claim 19 or 20 wherein the plant cell a cereal cell.

24. The method of claim 19 or 20 wherein the plant cell is selected from the group consisting of a maize, soybean, barley, alfalfa, sunflower, canola, soybean, cotton, peanut, sorghum, tobacco, sugarbeet, rice, wheat and Arabidopsis cell.

25. The method of claim 19 or 20 wherein the open reading frame is in an antisense orientation relative to the nucleotide sequence which alters transcription.

26. The method of claim 19 or 20 wherein the expression inhibits transcription or translation of endogenous plant nucleic acid sequences corresponding to the open reading frame.

27. The method of claim 19 wherein the open reading frame is expressed in an amount that is greater than the amount in a plant which does not comprise the expression cassette.

28. The method of claim 18 or 19 wherein the open reading frame encodes a protein.

29. The method of claim 28 wherein the protein encodes a regulatory product.

30. The method of claim 28 wherein the expression of the open reading frame confers insect resistance, bacterial resistance, fungal resistance, viral resistance, or nematode resistance.

31. A transformed plant prepared by the method of claim 20.

32. A product of the plant of claim 31 which comprises the expression cassette or the gene product encoded by the open reading frame.

33. The product of claim 32 which is selected from the group consisting of a seed, fruit, vegetable, transgenic plant, and a progeny plant.

34. A method to confer resistance or tolerance to a plant to a pathogen, comprising:
a) contacting plant cells with an expression cassette comprising a polynucleotide encoding a polypeptide that is substantially similar to a polypeptide encoded by an open reading frame comprising any one of SEQ ID NOs: 50, 139, 609, 4210, 3311, 3791, 2699, 3463, 3584, 4451 or 4595 so as to yield transformed cells; and
b) regenerating the transformed plant cells to provide a differentiated transformed plant, wherein the differentiated transformed plant expresses the polynucleotide in the cells of the plant in an amount effective to confer resistance or tolerance to the plant to a pathogen relative to a corresponding plant which does not comprise the expression cassette.

35. The method of claim 34 wherein the cells are monocot cells.

36. The method of claim 34 wherein the cells are dicot cells.

37. The method of claim 34 wherein the open reading frame encodes a DNA binding protein, hormone response protein, membrane protein, metabolic protein, transposon, receptor/kinase, phosphatase, stress protein, cell wall protein, lipid transfer protein, heat shock protein, protein processing protein, RNA processing protein, non-cell wall structural protein or a non-kinase signaling protein.

38. A transformed plant prepared by the method of claim 34.

39. A seed of the plant of claim 38.

40. A progeny plant of the plant of claim 39.

41. A method to identify a plant cell infected with a pathogen, comprising:
a) contacting isolated nucleic acid obtained from a plant cell suspected of being infected with a pathogen with at least one oligonucleotide under conditions effective to specifically amplify a nucleotide sequence corresponding to one of SEQ ID NOs: 50, 139, 609, 4210, 3311, 3791, 2699, 3463, 3584, 4451 or 4595 or a portion thereof, so as to yield an amplified product; and
b) detecting or determining the presence or amount of the amplified product, wherein the presence or amount of the amplified product is indicative of pathogen infection.

42. A method to identify a plant cell infected with a pathogen, comprising:
a) contacting a sample comprising polypeptides obtained from a plant cell suspected of being infected with a pathogen with an agent that specifically binds to a polypeptide that is substantially similar to a polypeptide encoded by an open reading frame comprising one of SEQ ID NOs: 50, 139, 609, 4210, 3311, 3791, 2699, 3463, 3584, 4451 or 4595 so as to form a complex; and
b) detecting or determining the presence or amount of the complex, wherein the presence or amount of the complex is indicative of pathogen infection.

43. A method to identify a plant cell infected with a pathogen, comprising:
a) contacting nucleic acid obtained from a plant cell suspected of being infected with a pathogen with a probe corresponding to a sequence selected from the group consisting of SEQ ID Nos. 50, 139, 609, 4210, 3311, 3791, 2699, 3463, 3584, 4451 or 4595 or a portion thereof, under stringent hybridization conditions to form a duplex; and
b) detecting or determining the presence or amount of the duplex, wherein the presence of a duplex is indicative of infection.
